(19) United States
(12) Patent Application Publication
(10) Pub. No.: US 2024/0382568 A1
(43) Pub. Date: Nov. 21, 2024

(54) ALKALINE PHOSPHATASE POLYPEPTIDES AND METHODS OF USE THEREOF

(71) Applicant: Alexion Pharmaceuticals, Inc., Boston, MA (US)

(72) Inventors: Walter C. VOEGTLI, Boston, MA (US); Yuhong WU, Boston, MA (US); Jonathan MONTELEONE, Boston, MA (US); Tatyana MEZHEBOVSKY, Boston, MA (US); Eric FALCONE, Boston, MA (US); Yang GUO, Boston, MA (US)

(21) Appl. No.: 18/796,936

(22) Filed: Aug. 7, 2024

Related U.S. Application Data
(63) Continuation of application No. 17/669,275, filed on Feb. 10, 2022, now Pat. No. 12,083,169.
(60) Provisional application No. 63/149,090, filed on Feb. 12, 2021.

(51) Int. Cl. A61K 38/46; A61K 47/02; A61K 47/22; A61K 47/26; A61P 19/08; C12N 9/16

(52) U.S. Cl. CPC A61K 38/465; A61K 47/02; A61K 47/22; A61K 47/26; A61P 19/08; C12N 9/16; C12Y 301/03001; C07K 2319/30

(57) ABSTRACT

Featured are pharmaceutical compositions that include a soluble alkaline phosphatase for treating bone mineralization disorders, such as hypophosphatasia (HPP), and symptoms thereof. The polypeptides include a soluble alkaline phosphatase (sALP) or fragment thereof, which is derived from a naturally occurring alkaline phosphatase (ALP).

### SEQUENCE LISTING

This application contains a Sequence Listing which has been filed electronically in Extensible Markup Language (XML) format and is hereby incorporated by reference in its entirety. Said XML copy, created on Aug. 7, 2024, is named 50694-094003_Sequence_Listing_8_7_24.XML and is 9,206 bytes in size.

### BACKGROUND

Hypophosphatasia (HPP) is a rare, heritable skeletal disease with an incidence of 1 per 100,000births for the most severe forms of the disease. The disorder typically results from loss-of-function mutations in the gene coding for tissue-nonspecific alkaline phosphatase (TNSALP). HPP exhibits a remarkable range of symptoms and severity, from premature tooth loss to almost complete absence of bone mineralization in utero. The presentation of HPP varies markedly among subjects and also varies markedly between subject ages. Many subjects with HPP display skeletal changes, short stature, chronic pain, painful lower limbs, gait disturbance, and premature, atraumatic tooth loss. Asfotase alfa (STRENSIQ®, Alexion Pharmaceuticals, Inc.), a recombinantly produced enzyme replacement therapy (ERT) that includes a soluble fragment of TNSALP, is the first ERT available to HPP subjects. Asfotase alfa has shown transformative effects on the most severe form of HPP, as evidenced by improvements in bone mineralization and density, as well as respiratory and motor function, cognitive development, and muscle strength (Whyte et al., New Engl. J. Med. 366:904-913, 2012).

### SUMMARY

A first aspect features a pharmaceutical composition containing an alkaline phosphatase polypeptide with at least 80% (e.g., at least 85%, 90%, 95%, 97%, 99%, or 100%) sequence identity to SEQ ID NO: 5 and a pharmaceutically acceptable carrier. The polypeptide may include at least one mutation selected from E108M, N213Q, and N286Q relative to the amino acid sequence of SEQ ID NO: 1 (e.g., the polypeptide may contain two or all three of these mutations). For example, the polypeptide has at least 80% (e.g., at least 85%, 90%, 95%, 97%, 99%, or 100%) sequence identity to the amino acid sequence of SEQ ID NO: 5 and contains at least one, two, or all three of the mutations selected from E108M, N213Q, and N286Q. The pharmaceutically acceptable carrier may include one or more of phosphate, proline, and sucrose. For example, the polypeptide may include or consist of the amino acid sequence of SEQ ID NO: 5.

In some embodiments, the alkaline phosphatase portion of polypeptide has at least 80% (e.g., at least 85%, 90%, 95%, 97%, 99%, or 100%) sequence identity to SEQ ID NO: 3. This polypeptide may further be connected to an Fc region (e.g., a IgG1, IgG2, IgG3, or IgG4 Fc region) and/or a polyaspartate region. In some embodiments, the polypeptide includes an IgG2/4 Fc region, e.g., having have at least 80% (e.g., at least 85%, 90%, 95%, 97%, 99%, or 100%) identify to SEQ ID NO: 4. The polyaspartate may include, e.g., 3, 4, 5, 6, 7, 8, 9, 10, 11, 12, 13, 14, 15, 16, 17, 18, 19, or 20 aspartate residues. In some embodiments, the polyaspartate includes ten aspartate residues (D10).

The composition may be formulated to contain a dosage of the alkaline phosphatase polypeptide of from about 0.1 mg/mL to about 200 mg/ml (e.g., about 1, 10, 20, 25, 50, 75, 100, 125, 150, 175, or 200 mg/mL. The composition may be formulated in a volume of about 0.1 mL to about 50 mL (e.g., about 0.1 to about 10 mL, e.g., about 0.1 mL, 0.2 mL, 0.3 mL, 0.4 mL, 0.5 mL, 0.6 mL, 0.7 mL, 0.8 mL, 0.9 mL, or 1.0 mL, e.g., about 1 mL to about 10 mL, e.g., about 2 mL, 3 mL, 4 mL, 5 mL, 6 mL, 7 mL, 8 mL, 9 mL, or 10 mL). In some embodiments, the composition is formulated in about 1 mL). For example, the composition may contain 100 mg/ml of an alkaline phosphatase polypeptide with at least 80% (e.g., at least 85%, 90%, 95%, 97%, or 99%) sequence identity to, or the sequence of, SEQ ID NO: 5 (e.g., the polypeptide contains at least one, two, or all three of the mutations selected from E108M, N213Q, and N286Q of SEQ ID NO: 5) and a pharmaceutically acceptable carrier.

The composition may include phosphate (e.g., sodium phosphate), e.g., in a concentration of from about 1 mM to about 100 mM, or from about 5 mM to about 20 mM, e.g., about 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM, e.g., about 10 mM. The composition may further include proline and/or sucrose. The composition may further include proline. The composition may further include sucrose. For example, the composition may include from about 1 mM to about 500 mM proline, e.g., from about 70 mM to about 280 mm, e.g., from about 50 mM to about 200 mM, e.g., about 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 300 mM, 400 mM, or 500 mM, e.g., about 140 mM proline and/or from about 1 mM to about 500 mM sucrose, e.g., from about 70 mM to about 280 mm, e.g., from about 50 mM to about 200 mM, e.g., about 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210, mM, 220 mM, 230 mM, 240 mM, 250 mM, 260 mM, 270 mM, 280 mM, 290 mM, 300 mM, 350 mM, 400 mM, 450 mM, or 500 mM, e.g., about 140 mM sucrose or about 210 mM sucrose. In some embodiments, the composition includes a molar ratio of proline: sucrose of about 1:0 to about 1:3, e.g., about 1:1. In some embodiments, the formulation includes from about 1 mM to about 500 mM proline (e.g., about 50 mM to about 200 mM proline, e.g., about 140 mM proline) and from about 1 mM to about 500 mM sucrose (e.g., about 40 mM to about 280 mM, e.g., about 50 mM to about 200 mM sucrose, e.g., about 140 mM or about 210 mM sucrose). In some embodiments, the formulation includes about 140 mM proline. In some embodiments, the formulation includes about 140 mM sucrose. In some embodiments, the formulation includes about 210 mM sucrose. In some embodiments, the formulation includes about 210 mM sucrose and does not include proline. In some embodiments, the formulation includes about 140 mM proline and about 140 mM sucrose. In some embodiments, the formulation includes about 140 mM proline and about 140 mM sucrose and about 10 mM phosphate (e.g., sodium phosphate). In some embodiments, the formulation includes about 210 mM sucrose and about 10 mM phosphate (e.g., sodium phosphate). The formulation may further include from about 0.01% to about 0.5% polyoxyethylene (20) sorbitan monooleate), e.g., from about 0.01% to about 0.1% polyoxyethylene (20) sorbitan monooleate, such as, e.g., about 0.05% polyoxyethylene (20) sorbitan monooleate. The polyoxyethylene (20) sorbitan monooleate may be, e.g., polysorbate 80 (PS80). In some embodiments, the composition is formulated at a pH of about pH 7.0 to about pH 7.6 (e.g., about pH 7.1, 7.2, 7.3, 7.4, 7.5, or 7.6, e.g., about pH 7.3). In some embodiments, the composition includes about 10 mM phosphate, about 140 mM proline, about 140 mM sucrose, and about 0.05% polyoxyethylene (20) sorbitan monooleate (e.g., PS80) at a pH of about 7.3.

The composition may be a pharmaceutical composition formulated as a solution containing the polypeptide (e.g., the polypeptide of SEQ ID NO: 5 and variants thereof with at least about 85% sequence identity thereto (e.g., the polypeptide is one that contains one, two, or all three of the mutations selected from E108M, N213Q, and N286Q of SEQ ID NO: 5)). The pharmaceutical composition may contain the polypeptide in an amount of, e.g., about 0.1 mg/mL to about 200 mg/mL, such as about 100 mg/mL. The pharmaceutical composition may be formulated for subcutaneous administration, e.g., at a dosage of the polypeptide of from about 0.1 mg/mL to about 10 mg/mL. The composition may be formulated in a solution in a volume of about 0.1 mL to about 50 mL (e.g., about 0.1 to about 10 mL, e.g., about 0.1 mL, 0.2 mL, 0.3 mL, 0.4 mL, 0.5 mL, 0.6 mL, 0.7 mL, 0.8 mL, 0.9 mL, or 1.0 mL, e.g., about 1 mL to about 10 mL, e.g., about 2 mL, 3 mL, 4 mL, 5 mL, 6 mL, 7 mL, 8 mL, 9 mL, or 10 mL). In some embodiments, the composition is formulated in about 1 mL. The solution may contain about 10 mM phosphate, about 140 mM proline, about 140 mM sucrose, and about 0.05% polyoxyethylene (20) sorbitan monooleate (e.g., PS80) at a pH of about 7.3.

Also featured is a vial containing the pharmaceutical composition as described herein. The vial may contain a solution (e.g., about 10 mM sodium phosphate, about 140 mM proline, about 140 mM sucrose, and about 0.05% polyoxyethylene (20) sorbitan monooleate (e.g., PS80) at a pH of about 7.3) containing the polypeptide (e.g., in an amount of about 0.1 mg to about 1.0 g (e.g., about 10 mg to about 200 mg)) in a volume of, e.g., from about 0.1 mL to about 10 mL (e.g., about 1 mL). The vial may contain the polypeptide in an amount of, e.g., about 0.1 mg/mL to about 500 mg/mL, about 1 mg/ml to about 200 mg/mL, about 50 mg/mL to about 150 mg/mL, or about 100 mg/mL. The polypeptide may have the amino acid sequence of SEQ ID NO: 5 or a variant thereof with at least 85% sequence identity thereto (e.g., the polypeptide is one that contains one, two, or all three of the mutations selected from E108M, N213Q, and N286Q of SEQ ID NO: 5).

A second aspect features a method of treating a bone mineralization disorder or a disease with bone manifestations (e.g., a disease selected from the group consisting of hypophosphatasia (HPP), bone fracture, osteoporosis, sclerosteosis, chondrocalcinosis, hypotonia, Duchenne's muscular dystrophy, tracheobronchomalacia, seizure, neurofibromatosis (e.g., NF-1), and craniosynostosis, or one or more symptoms thereof, in a subject (e.g., a human subject) in need thereof by administering to the subject the pharmaceutical composition of the first aspect. The composition may be administered in an amount and for a duration sufficient to treat the disease or to alleviate one or more symptoms thereof. The treatment may enhance bone formation in the subject. The polypeptide may be used to treat muscle weakness.

The polypeptide or a pharmaceutical composition containing the same may be administered at a dosage of from about 0.01 mg/kg to about 60 mg/kg (e.g., from about 0.1 mg/kg to about 50 mg/kg, e.g., from about 0.1 mg/kg to about 20 mg/kg, or, e.g., from about 0.1 mg/kg to about 10 mg/kg). The polypeptide may be administered once per day, week, month, or year (e.g., once per week). In some embodiments, the polypeptide is administered one or more times every 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The polypeptide may be administered once a week, once every two weeks, once every three weeks, once every four weeks or longer. The polypeptide may be administered at a dosage of from about 0.01 mg/kg/week to about 50 mg/kg/week (e.g., from about 0.01 mg/kg/week to about 40 mg/kg/week, e.g., from about 0.1 mg/kg/week to about 20 mg/kg/week, or, e.g., from about 0.1 mg/kg/week to about 10 mg/kg/week). The polypeptide may be administered for at least one day, one week, one month, one year, or longer (e.g., for the life of the subject).

The polypeptide or a pharmaceutical composition containing the same may be administered subcutaneously, intravenously, intramuscularly, sublingually, intrathecally, or intradermally. In particular, the polypeptide, or a composition containing the polypeptide, may be administered by subcutaneous or intravenous administration.

In some embodiments, the pharmaceutical composition is administered subcutaneously (e.g., in the abdomen or thigh). For example, from about 10 mg to about 100 mg (e.g., about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, e.g., about 15 mg, 45 mg, or 90 mg, such as about 15 mg, 45 mg, or 90 mg) may be administered to the subject subcutaneously, e.g., once or twice per week, e.g., for at least 1, 2, 3, 4, 5, 6, 7, 8, 9, 10, or more weeks, or for longer (e.g., for the life of the subject). The composition may be administered in a volume of, e.g., about 5 mL or less (e.g., 4.0 mL, 3.0 mL, 2.0 mL, 1.0 mL, 0.9 mL, 0.8 mL, 0.7 mL, 0.6 mL, 0.5 mL, 0.4 mL, 0.3 mL, 0.2 mL, or 0.1 mL, or in a volume in a range of from about 5 mL to about 0.1 mL).

In some embodiments, the pharmaceutical composition is administered intravenously (IV). For example, from about 10 mg to about 100 mg (e.g., about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, such as, e.g., about 15 mg, 45 mg, or 90 mg) may be administered to the subject intravenously, e.g., once or twice per week, e.g., for at least 1, 2, 3, 4, 5, 6, 7, 8, 9, 10, or more weeks (e.g., for 3 weeks), or for longer (e.g., for the life of the subject). For example, the pharmaceutical composition is administered by IV in a volume of about 5 mL or less (e.g., 4.0 mL, 3.0 mL, 2.0 mL, 1.0 mL, 0.9 mL, 0.8 mL, 0.7 mL, 0.6 mL, 0.5 mL, 0.4 mL, 0.3 mL, 0.2 mL, or 0.1 mL, or in a volume in a range of from about 5 mL to about 0.1 mL).

In some embodiments, the pharmaceutical composition is administered intravenously and subcutaneously (e.g., in the abdomen or thigh). For example, the pharmaceutical composition is administered in a treatment regimen that combines intravenous and subcutaneous (e.g., in the abdomen or thigh) administration. For example, the composition may first be administered to a subject intravenously in a single dose in an amount of from about 10 mg to about 100 mg (e.g., about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, such as, e.g., about 15 mg, 45 mg, or 90 mg) followed by subcutaneous administration (e.g., in the abdomen or thigh) to the subject in one or more doses over time. For example, the subcutaneous dose may be from about 10 mg to about 100 mg (e.g., about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, e.g., about 15 mg, 45 mg, or 90 mg, such as about 15 mg, 45 mg, or 90 mg). The subcutaneous doses may be administered, e.g., once or twice per week, once every two weeks, once every three weeks, or once every four weeks, e.g., for at least 1, 2, 3, 4, 5, 6, 7, 8, 9, 10, 20, 30, 40, or 50 or more weeks, or for longer (e.g., for 1-10 years, or for the life of the subject). The intravenous and subcutaneous doses may be administered in a volume of, e.g., about 5 mL or less (e.g., 4.0 mL, 3.0 mL, 2.0 mL, 1.0 mL, 0.9 mL, 0.8 mL, 0.7 mL, 0.6 mL, 0.5 mL, 0.4 mL, 0.3 mL, 0.2 mL, or 0.1 mL, or in a volume in a range of from about 5 mL to about 0.1 mL).

The subject may be a human subject, such as a neonate, an infant, a child, an adolescent, or an adult.

In some embodiments, the TSAC of the recombinant alkaline phosphatase polypeptide is about 1.0 mol/mol to about 6.0 mol/mol. In some embodiments, the TSAC is about 1.2 mol/mol to about 6.0 mol/mol recombinant alkaline phosphatase. In some embodiments, the TSAC is about 1.5 mol/mol to about 6.0 mol/mol recombinant alkaline phosphatase. In some embodiments, the TSAC is about 3.0 mol/mol to about 6.0 mol/mol recombinant alkaline phosphatase. In some embodiments, the TSAC is about 3.2 mol/mol to about 5.9 mol/mol recombinant alkaline phosphatase. In some embodiments, the TSAC is about 0.9 mol/mol, about 1.0 mol/mol, about 1.1 mol/mol, about 1.2 mol/mol, about 1.3 mol/mol, about 1.4 mol/mol, about 1.5 mol/mol, about 1.6 mol/mol, about 1.7 mol/mol, about 1.8 mol/mol, about 1.9 mol/mol, about 2.0 mol/mol, about 2.1 mol/mol, about 2.2 mol/mol, about 2.3 mol/mol, about 2.4 mol/mol, about 2.5 mol/mol, about 2.6 mol/mol, about 2.7 mol/mol, about 2.8 mol/mol, about 2.9 mol/mol, about 3.0 mol/mol, about 3.1 mol/mol, about 3.2 mol/mol, about 3.3 mol/mol, about 3.4 mol/mol, about 3.5 mol/mol, about 3.6 mol/mol, about 3.7 mol/mol, about 3.8 mol/mol, about 3.9 mol/mol, about 4.0 mol/mol, about 4.1 mol/mol, about 4.2 mol/mol, about 4.3 mol/mol, about 4.4 mol/mol, about 4.5 mol/mol, about 4.6 mol/mol, about 4.7 mol/mol, about 4.8 mol/mol, about 4.9 mol/mol, about 5.0 mol/mol, about 5.1 mol/mol, about 5.2 mol/mol, about 5.3 mol/mol, about 5.4 mol/mol, about 5.5 mol/mol, about 5.6 mol/mol, about 5.7 mol/mol, about 5.8 mol/mol, about 5.9 mol/mol, or about 6.0 mol/mol recombinant alkaline phosphatase. In some embodiments, the TSAC is about 3.2 mol/mol recombinant alkaline phosphatase. In some embodiments, the TSAC is about 5.0 mol/mol recombinant alkaline phosphatase. In some embodiments, the TSAC is about 5.9 mol/mol recombinant alkaline phosphatase.

In some embodiments, the composition includes about 10 mM phosphate, about 140 mM proline, about 140 mM sucrose, about 0.05% polyoxyethylene (20) sorbitan monooleate (e.g., PS80) at a pH of about 7.3, and a TSAC value of from about 3.0 mol/mol to about 6.0 mol/mol.

In some embodiments, the method produces an AUC₀₋₁₆₈ₕ of from about 50 μg×hour/mL to about 4000 μg×hour/mL in the blood of the subject. For example, the method may produce an AUC₀₋₁₆₈ₕ of from about 1000 μg×hour/mL to about 3000 μg×hour/mL in the blood of the subject. In some embodiments, the method produces a Cₘₐₓ of from about 0.5 μg/mL to about 25 μg/mL in the blood of the subject. For example, the method may produce a Cₘₐₓ of from about 0.6 μg/mL to about 20 μg/mL in the blood of the subject.

### Definitions

The term “about” means ±10% of the recited value. All measurements reported herein are understood to be modified by the term “about,” whether or not the term is explicitly used, unless explicitly stated otherwise.

The term “bone-targeting moiety” means an amino acid sequence of at least 3 amino acid residues in length having a sufficient affinity to bone matrix such that the bone-targeting moiety, taken alone, has an in vivo binding affinity to the bone matrix that is at least about 1×10⁻⁵ M or greater, e.g., about 10⁻⁶ M, about 10⁻⁷ M, about 10⁻⁸ M, about 1⁻⁹ M, or greater).

The term “catalytically competent,” as used herein, refers to a sALP that hydrolyzes the bone mineralization inhibitor inorganic pyrophosphate (PPi) to provide inorganic phosphate (Pi), thereby decreasing the extracellular concentrations of PPi. Thus, a catalytically competent sALP improves skeletal mineralization by regulating the concentration of PPi.

The term “Fc” means a fragment crystallizable region of an immunoglobulin, e.g., IgG1, IgG2,IgG3, or IgG4, including the CH2 and CH3 domains of the immunoglobulin heavy chain. Fc may also include any portion of the hinge region joining the Fab and Fc regions. The Fc can be derived from any mammal, including a human, and may be post-translationally modified (e.g., by glycosylation or sialylation). In a non-limiting example, Fc can be the fragment crystallizable region of human IgG2/4 of SEQ ID NO: 4.

By “fragment” is meant a portion of a polypeptide or nucleic acid molecule that contains, preferably, at least 10%, 20%, 30%, 40%, 50%, 60%, 70%, 75%, 80%, 85%, 90%, 95%, 96%, 97%, 98%, 99%, or more of the entire length of the reference nucleic acid molecule or polypeptide. A fragment may contain, e.g., 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 500, 600, 700, 800, 900, 1,000, 1100, 1200, 1300, 1400, 1500, 1600, 1700, 1800, 1900, 2000, 2100, or more nucleotides, up to the entire length of the nucleic acid molecule, or 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 400, 500, 600, 700, or more amino acid residues, up to the entire length of the polypeptide.

The terms “hypophosphatasia” and “HPP,” as used herein, refer to a rare, heritable skeletal disorder caused by, e.g., one or more loss-of-function mutations in the ALPL (alkaline phosphatase, liver/bone/kidney) gene, which encodes tissue-nonspecific alkaline phosphatase (TNSALP). HPP may be further characterized as infantile HPP, childhood HPP, perinatal HPP (e.g., benign perinatal HPP or lethal perinatal HPP), odonto-HPP, adolescent HPP, or adult HPP. For instance, “childhood HPP” describes a subject having HPP that is from about 5 years of age to about 12 years, “adolescent HPP” describes a subject having HPP that is from about 13 years of age to about 17 years, and “adult HPP” describes a subject having HPP that is about 18 years of age or older. The term “adult HPP,” as used herein, refers to a condition or phenotype characterized by the presence of one or more of the following symptoms: elevated blood and/or urine levels of inorganic pyrophosphate (PPi), hypomineralization, hypercalciuria, one or more skeletal deformities, hypotonia, muscle weakness, rheumatoid complications, waddling gait, ambulatory difficulties, bone pain, pain, bone fracture, calcium pyrophosphate dihydrate crystal deposition, pseudogout, arthritis, pyrophosphate arthropathy, chondrocalcinosis, calcific periarthritis, and pseudofracture. The term “adolescent HPP,” as used herein, refers to a condition or phenotype characterized by the presence of one or more of the following symptoms: elevated blood or urine levels of PPi, PEA, or PLP; osteomalacia, one or more skeletal deformities, hypotonia, muscle weakness, rheumatoid complications, arthritis, pseudogout, waddling gait, ambulatory difficulties, bone pain, pain, premature loss of teeth, hypomineralization, pulmonary hypoplasia, respiratory insufficiency, seizures, hypercalciuria, short stature, and growth delay. The term “childhood HPP,” as used herein, refers to refers to a condition or phenotype characterized by the presence of one or more of the following symptoms: elevated blood or urine levels of PPi, PEA, or PLP; rickets, rachitic ribs, one or more skeletal deformities, hypotonia, muscle weakness, rheumatoid complications, arthritis, pseudogout, waddling gait, ambulatory difficulties, bone pain, pain, premature loss of teeth, hypomineralization, delayed motor development, seizures, hypercalciuria, short stature, bone fracture, pseudofracture, and growth delay.

The term “nucleic acid” or “nucleic acid molecule” means a polymeric molecule, e.g., RNA or DNA, having a sequence of two or more covalently bonded, naturally occurring or modified, nucleotides. The nucleic acid molecule may be, e.g., single or double stranded, and may include modified or unmodified nucleotides, or mixtures or combinations thereof. Various salts, mixed salts, and free acid forms of nucleic acid molecules are also included.

By “treating,” “treat,” and “treatment” is meant the medical management of a subject with the intent to cure, ameliorate, stabilize, reduce the likelihood of, or prevent a disease condition, such as HPP (e.g., child, adolescent, or adult HPP), or one or more symptoms thereof and/or the management of a subject exhibiting or likely to have a disease condition, such as HPP, e.g., by administering a pharmaceutical composition (e.g., an sALP as described herein). Treating (and the other forms used herein) includes active treatment, that is, treatment directed specifically toward the improvement or associated with the cure of a disease, pathological condition, disorder, or event, and also includes causal treatment, that is, treatment directed toward removal of the cause of the associated disease, pathological condition, disorder, or event. In addition, this term includes palliative treatment, that is, treatment designed for the relief or improvement of at least one symptom rather than the curing of the disease, pathological condition, disorder, or event; symptomatic treatment, that is, treatment directed toward constitutional symptoms of the associated disease, pathological condition, disorder, or event; preventative treatment, that is, treatment directed to minimizing or partially or completely inhibiting the development of the associated disease, pathological condition, disorder, or event, e.g., in a subject who is not yet ill, but who is susceptible to, or otherwise at risk of, a particular disease, pathological condition, disorder, or event; and supportive treatment, that is, treatment employed to supplement another specific therapy directed toward the improvement of the associated disease, pathological condition, disorder, or event.

The terms “peptide,” “polypeptide,” and “protein” are used interchangeably and refer to any chain of two or more natural or unnatural amino acid residues, regardless of post-translational modification (e.g., glycosylation, sialylation, or phosphorylation), constituting all or part of a naturally-occurring or non-naturally occurring polypeptide or peptide, as is described herein.

The terms “sALP,” “soluble alkaline phosphatase,” and “extracellular domain of an alkaline phosphatase” are used interchangeably (unless the context indicates otherwise) to mean a soluble, non-membrane-bound alkaline phosphatase or a biologically active fragment or variant thereof. sALPs include, for example, an alkaline phosphatase lacking a C-terminal GPI signal sequence, and additional variants and analogs thereof which retain alkaline phosphatase activity, e.g., the ability to hydrolyze PPi or other natural or artificial substrate(s). This includes soluble fragments corresponding to the extracellular domains of TNSALP, PALP, GLALP, and IALP and biologically active fragments or variants thereof, unless specified otherwise. A mature sALP lacks the GPI membrane anchor and the signal peptide, which is cleaved during processing.

The terms “ALP” and “alkaline phosphatase” refer to a naturally occurring alkaline phosphatase, such as TNSALP, PALP, GLALP, and IALP, which is able to hydrolyze PPi or other natural or artificial substrates.

The term “pharmaceutically acceptable carrier” or “pharmaceutically acceptable excipient” is meant a carrier or excipient that is physiologically acceptable to the treated subject while retaining the therapeutic properties of the compound with which it is administered. One exemplary pharmaceutically acceptable carrier substance is physiological saline. Other physiologically acceptable carriers and their formulations are known to those skilled in the art and described, for example, in Remington's Pharmaceutical Sciences (Remington: The Science and Practice of Pharmacy, 22nd Ed., Allen, Ed. 2012).

The term “pharmaceutical composition” means a composition containing a polypeptide or nucleic acid molecule as described herein formulated with a pharmaceutically acceptable excipient, and includes those that are manufactured or sold with the approval of a governmental regulatory agency as part of a therapeutic regimen for the treatment or prevention of a disease or event in a subject. Pharmaceutical compositions can be formulated, for example, for subcutaneous administration (e.g., in the abdomen or thigh), intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

The term “subject” means a mammal, including, but not limited to, a human or non-human mammal, such as a bovine, equine, canine, ovine, or feline.

The term “therapeutically effective amount” means an amount of a polypeptide or nucleic acid molecule described herein that is sufficient to substantially treat, prevent, delay, suppress, or arrest any symptom of a disease or condition described herein, particularly HPP. A therapeutically effective amount of a composition described herein may depend on the severity of the disorder being treated and the condition, weight, and general state of the subject and can be determined by an ordinarily-skilled artisan with consideration of such factors. A therapeutically effective amount of a composition described herein can be administered to a subject in a single dose or in multiple doses administered over a period of time.

The terms “Total Sialic Acid Content” or “TSAC,” as used herein, refer to the amount of sialic acid (a carbohydrate) on a particular protein molecule. It is expressed as moles sialic acid incorporated per mole of protein, or “mol/mol.” TSAC concentration is measured during the purification process. For example, one method of TSAC quantitation is where TSAC is released from the alkaline phosphatase using acid hydrolysis, and the released TSAC is subsequently detected via electrochemical detection using high-performance anion-exchange chromatography with pulsed amperometric detection technique (“HPAE-PAD”).

The term “sialic acid” refers generally to N- or O-substituted derivatives of neuraminic acid, a monosaccharide with a nine-carbon backbone. Sialic acid may also refer specifically to the compound N-acetylneuraminic acid and is sometimes abbreviated as Neu5Ac or NANA. Presence of sialic acid may affect absorption, serum half-life, and clearance of glycoproteins from the serum, as well as physical, chemical, and immunogenic properties of the glycoprotein. In some embodiments of the present disclosure, sialic acid associated with alkaline phosphatases, e.g., ALP201, impacts in vivo exposure and the half-life of the molecule in physiological conditions. In some embodiments, precise and predictable control of total sialic acid content (TSAC) of an alkaline phosphatase serves as a quality control attribute.

As used herein, when a polypeptide or nucleic acid sequence is referred to as having “at least X % sequence identity” to a reference sequence, it is meant that at least X percent of the amino acid residues or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith et al., J. Mol. Biol. 147:195-7, 1981) and BLAST (Basic Local Alignment Search Tool; Altschul et al., J. Mol. Biol. 215:403-10, 1990). These and other alignment algorithms are accessible using publicly available computer software such as “Best Fit” (Smith and Waterman, Advances in Applied Mathematics, 482-489, 1981) as incorporated into GENEMATCHER PLUS™ (Schwarz and Dayhof, Atlas of Protein Sequence and Structure, Dayhoff, M. O., Ed., pp 353-358, 1979), BLAST, BLAST-2, BLAST-P, BLAST-N, BLAST-X, WU-BLAST-2, ALIGN, ALIGN-2, CLUSTAL, or Megalign (DNASTAR). In addition, those skilled in the art can determine appropriate parameters for measuring alignment, including any algorithms needed to achieve optimal alignment over the length of the sequences being compared.

The words “preferred” and “preferably” refer to embodiments of the disclosed compounds, compositions and methods that may afford certain benefits, under certain circumstances. However, other embodiments may also be preferred, under the same or other circumstances. Furthermore, the recitation of one or more preferred embodiments does not imply that other embodiments are not useful, and the use of this term is not intended to exclude other embodiments from the scope of the disclosure.

For any method disclosed herein that includes discrete steps, the steps may be conducted in any feasible order; also, as appropriate, any combination of two or more steps may be conducted simultaneously.

The above summary is not intended to describe each disclosed embodiment or every implementation of disclosed compounds, compositions and methods. The description that follows more particularly exemplifies illustrative embodiments. In several places throughout the application, guidance may be provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

### BRIEF DESCRIPTION OF THE DRAWINGS

FIG. 1 is a set of saturation curves showing the relationship of increasing PPi levels with rate of PPi hydrolysis for ALP201 (SEQ ID NO: 5; left) and asfotase alfa (SEQ ID NO: 6; right).

FIG. 2 is a set of saturation curves showing the relationship of increasing PLP levels with rate of PLP hydrolysis for ALP201 (left) and asfotase alfa (right).

FIG. 3 is a graph showing active alkaline phosphatase enzyme concentrations in Akp2GW (−/−) mouse plasma at study end by dosing group. Abbreviations: q1w=once weekly; q2d=every 2 days; qd=every day.

FIG. 4 is a graph showing Day 36/37 end-of-study mouse femur tissue alkaline phosphatase activity levels. Abbreviations: q2d=every 2 days; qd=every day.

FIG. 5 is a set of representative survival curves for Akp2GW (−/−) mice treated with ALP201 and asfotase alfa in 36-day efficacy studies. Abbreviations: MED=minimum efficacious dose; PBS=phosphate buffered saline; q1w=once weekly; q2d=every 2 days; qd=every day.

FIG. 6 is a graph showing a comparison of ALP201 and asfotase alfa dose-response modeling results. Abbreviations: ALP=alkaline phosphatase; D-R=dose response; MED=minimum efficacious dose; POC=proof of concept.

FIG. 7 is a graph showing simulated human ALP201 concentration-time profiles for each first-in-human (FIH) cohorts. Gray region represents the 90% prediction interval, i.e., 5% to 95% range; black solid line is the median of the simulated concentration-time profiles; LLOQ is the horizontal dashed line for the PK assay; Abbreviations: LLOQ=lower limit of quantitation (0.15 μg/mL).

FIGS. 8A and 8B are graphs showing mean (SD) ALP201 active plasma concentration versus time profiles following intravenous (FIG. 8A) and subcutaneous (FIG. 8B) administration to male C57BL/6 mice.

FIGS. 9A and 9B are graphs showing mean (SD) ALP201 active plasma concentration versus time profiles for ALP201 (lot with TSAC=5.9) following intravenous (FIG. 9A) and subcutaneous (FIG. 9B) administration to male C57BI/6 mice.

FIGS. 10A and 10B are graphs showing mean (SD) ALP201 active plasma concentration versus time profiles for ALP201 (lot with TSAC=5.0) following intravenous (FIG. 10A) and subcutaneous (FIG. 10B) administration to male C57BI/6 mice.

FIGS. 11A and 11B are graphs showing mean (SD) ALP201 active plasma concentration versus time profiles for ALP201 (lot with TSAC=3.2) following intravenous (FIG. 11A) and subcutaneous (FIG. 11B) administration to male C57BI/6 mice.

FIG. 12 is a graph showing mean (SD) ALP201 active plasma concentration versus time profiles for ALP201 TSAC values (3.2, 5.0, and 5.9) following intravenous administration to male C57BL/6 mice.

FIG. 13 is a graph showing mean (SD) ALP201 active plasma concentration versus time profiles for ALP201 TSAC variants following subcutaneous administration to male C57BL/6 mice.

FIG. 14 is a graph showing a comparison of ALP201 and asfotase alfa mean (±SD) plasma concentration versus time profiles after single IV and SC administration in rats. Abbreviations: IV intravenous; SC=subcutaneous, SD=standard deviation. Asfotase alfa source data: pooled sex, IV and SC at 3 mg/kg.

FIG. 15 is a graph showing a comparison of ALP201 and asfotase alfa mean (±SD) plasma concentration versus time profiles after single IV and SC administration in monkeys.

FIG. 16 is a graph showing a comparison of ALP201 and asfotase alfa (STRENSIQ®) dose-response modeling. Abbreviations: ALP=alkaline phosphatase; D-R=dose response; MED=minimum efficacious dose; POC=proof of concept.

### DETAILED DESCRIPTION

Featured are soluble alkaline phosphatases polypeptides (e.g., those having the sequence of SEQ ID NO: 5 and variants thereof with up to 80% or more sequence identity thereto, in which the polypeptides contain one, two, or all three of the mutations selected from E108M, N213Q, and N286Q of SEQ ID NO: 5), fragments thereof, and fusion proteins thereof, nucleic acid molecules encoding the same, and methods of using the polypeptides and nucleic acid molecules for treating a disease, such as a bone mineralization disorder, for example hypophosphatasia (HPP), or one or more symptoms thereof. The polypeptides include a soluble alkaline phosphatase (sALP) or fragment thereof, which is derived from a naturally occurring alkaline phosphatase (ALP). Alkaline phosphatases include various isozymes that are differentially expressed in different tissues. Four major ALP isozymes include tissue non-specific alkaline phosphatase (TNSALP), placental alkaline phosphatase (PALP), germ line alkaline phosphatase (GLALP), and intestinal alkaline phosphatase (IALP). Accordingly, featured are proteins derived from these ALP isozymes.

The polypeptides described herein are formulated into pharmaceutical compositions containing for example, one or more of phosphate, proline, and sucrose. These components impart beneficial features for the polypeptide, such as improved stability, reduced aggregation, reduction of truncated protein products, and increased purity of desired protein products in the formulation.

HPP is a rare, heritable skeletal disease with an incidence of 1 per 100,000 births for the most severe forms of the disease. The disorder typically results from loss-of-function mutations in the gene coding for TNSALP. HPP exhibits a remarkable range of symptoms and severity, from premature tooth loss to almost complete absence of bone mineralization in utero. The presentation of HPP varies markedly among subjects and also varies markedly between subject ages. Many subjects with HPP display skeletal changes, short stature, chronic pain, painful lower limbs, gait disturbance, and premature, atraumatic tooth loss. Due to the loss-of-function mutation in the endogenous TNSALP, a subject with HPP requires functional ALP activity of the polypeptides described herein to restore the native ALP activity and provide normal bone matrix mineralization.

### Soluble Alkaline Phosphatase Polypeptides

The polypeptides described herein include a soluble alkaline phosphatase (sALP), such as a mutant tissue nonspecific alkaline phosphatase (TNSALP) or fragment thereof. The sALP may be fused to an Fc region and a polyaspartate of sequence n (“Dn”, in which n equals, e.g., 3-20) (sALP-Fc-Dn). The polypeptide may include a human TNSALP, such as a soluble fragment of a human TNSALP (e.g., residues 1-491 or 1-485 of SEQ ID NO: 1). The polypeptide may include one, two, or all three of the mutations E108M, N213Q, and/or N286Q relative to SEQ ID NO: 1. For example, the sALP may have at least 80% (e.g., at least 85%, 90%, 95%, 97%, 99%, or 100%) identify to SEQ ID NO: 2 or 3. The Fc region may be an IgG2/4 Fc region. For example, the Fc region may have at least 80% (e.g., at least 85%, 90%, 95%, 97%, 99%, or 100%) identify to SEQ ID NO: 4. The polyaspartate may include, e.g., 3, 4, 5, 6, 7, 8, 9, 10, 11, 12, 13, 14, 15, 16, 17, 18, 19, or 20 aspartate residues. In some embodiments, the polyaspartate includes ten aspartate residues (D10). In some embodiments, the polypeptide has at least 80% (e.g., at least 85%, 90%, 95%, 97%, 99%, or 100%) identify to SEQ ID NO: 5. For example, the polypeptide may include or consist of the polypeptide of SEQ ID NO: 5. The polypeptide may consist of SEQ ID NO: 5.

In some embodiments, the alkaline phosphatase portion of polypeptide has at least 80% (e.g., at least 85%, 90%, 95%, 97%, 99%, or 100%) sequence identity to SEQ ID NO: 3. This polypeptide may further be connected to an Fc region (e.g., a IgG1, IgG2, IgG3, or IgG4 Fc region) and/or a polyaspartate region. In some embodiments, the polypeptide includes an IgG2/4 Fc region, e.g., having have at least 80% (e.g., at least 85%, 90%, 95%, 97%, 99%, or 100%) identify to SEQ ID NO: 4. The polyaspartate may include, e.g., 3, 4, 5, 6, 7, 8, 9, 10, 11, 12, 13, 14, 15, 16, 17, 18, 19, or 20 aspartate residues. In some embodiments, the polyaspartate includes ten aspartate residues (D10).

Human TNSALP lacking a signal peptide
(UniProt P05186.4)
SEQ ID NO: 1
LVPEKEKDPKYVVRDQAQETLKYALELQKLNTNVA
KNVIMFLGDGMGVSTVTAARILKGQLHHNPGEETR
LEMDKFPFVALSKTYNTNAQVPDSAGTATAYLCGV
KANEGTVGVSAATERSRCNTTQGNEVTSILRWAKD
AGKSVGIVTTTRVNHATPSAAYAHSADRDWYSDNE
MPPEALSQGCKDIAYQLMHNIRDIDVIMGGGRKYM
YPKNKTDVEYESDEKARGTRLDGLDLVDTVVKSFK
PRYKHSHFIWNRTELLTLDPHNVDYLLGLFEPGDM
QYELNRNNVTDPSLSEMVVVAIQILRKNPKGFFLL
VEGGRIDHGHHEGKAKQALHEAVEMDRAIGQAGSL
TSSEDTLTVVTADHSHVFTFGGYTPRGNSIFGLAP
MLSDTDKKPFTAILYGNGPGYKVVGGERENVSMVD
YAHNNYQAQSAVPLRHETHGGEDVAVFSKGPMAHL
LHGVHEQNYVPHVMAYAACIGANLGHCAPASSAGS
LAAGPLLLALALYPLSVLF
Human TNSALP (1-485; E108M, N213Q, N286Q)
SEQ ID NO: 2
LVPEKEKDPKYWRDQAQETLKYALELQKLNTNVAK
NVIMFLGDGMGVSTVTAARILKGQLHHNPGEETRL
EMDKFPFVALSKTYNTNAQVPDSAGTATAYLCGVK
ANMGTVGVSAATERSRCNTTQGNEVTSILRWAKDA
GKSVGIVTTTRVNHATPSAAYAHSADRDWYSDNEM
PPEALSQGCKDIAYQLMHNIRDIDVIMGGGRKYMY
PKQKTDVEYESDEKARGTRLDGLDLVDTWKSFKPR
YKHSHFIWNRTELLTLDPHNVDYLLGLFEPGDMQY
ELNRNQVTDPSLSEMVVVAIQILRKNPKGFFLLVE
GGRIDHGHHEGKAKQALHEAVEMDRAIGQAGSLTS
SEDTLTVVTADHSHVFTFGGYTPRGNSIFGLAPML
SDTDKKPFTAILYGNGPGYKVVGGERENVSMVDYA
HNNYQAQSAVPLRHETHGGEDVAVFSKGPMAHLLH
GVHEQNYVPHVMAYAACIGANLGHCAPASS
Human TNSALP (1-491; E108M, N213Q, N286Q)
SEQ ID NO: 3
LVPEKEKDPKYWRDQAQETLKYALELQKLNTNVAK
NVIMFLGDGMGVSTVTAARILKGQLHHNPGEETRL
EMDKFPFVALSKTYNTNAQVPDSAGTATAYLCGVK
ANMGTVGVSAATERSRCNTTQGNEVTSILRWAKDA
GKSVGIVTTTRVNHATPSAAYAHSADRDWYSDNEM
PPEALSQGCKDIAYQLMHNIRDIDVIMGGGRKYMY
PKQKTDVEYESDEKARGTRLDGLDLVDTWKSFKPR
YKHSHFIWNRTELLTLDPHNVDYLLGLFEPGDMQY
ELNRNQVTDPSLSEMVVVAIQILRKNPKGFFLLVE
GGRIDHGHHEGKAKQALHEAVEMDRAIGQAGSLTS
SEDTLTVVTADHSHVFTFGGYTPRGNSIFGLAPML
SDTDKKPFTAILYGNGPGYKVVGGERENVSMVDYA
HNNYQAQSAVPLRHETHGGEDVAVFSKGPMAHLLH
GVHEQNYVPHVMAYAACIGANLGHCAPASSAGSLA
A
IgG2/4 Fc
SEQ ID NO: 4
VECPPCPAPPVAGPSVFLFPPKPKDTLMISRTPEV
TCVVVDVSQEDPEVQFNWYVDGVEVHNAKTKPREE
QFNSTYRVVSVLTVLHQDWLNGKEYKCKVSNKGLP
SSIEKTISKAKGQPREPQVYTLPPSQEEMTKNQVS
LTCLVKGFYPSDIAVEWESNGQPENNYKTTPPVLD
SDGSFFLYSRLTVDKSRWQEGNVFSCSVMHEALHN
HYTQKSLSLSLGK
ALP201
SEQ ID NO: 5
LVPEKEKDPKYWRDQAQETLKYALELQKLNTNVAK
NVIMFLGDGMGVSTVTAARILKGQLHHNPGEETRL
EMDKFPFVALSKTYNTNAQVPDSAGTATAYLCGVK
ANMGTVGVSAATERSRCNTTQGNEVTSILRWAKDA
GKSVGIVTTTRVNHATPSAAYAHSADRDWYSDNEM
PPEALSQGCKDIAYQLMHNIRDIDVIMGGGRKYMY
PKQKTDVEYESDEKARGTRLDGLDLVDTWKSFKPR
YKHSHFIWNRTELLTLDPHNVDYLLGLFEPGDMQY
ELNRNQVTDPSLSEMVVVAIQILRKNPKGFFLLVE
GGRIDHGHHEGKAKQALHEAVEMDRAIGQAGSLTS
SEDTLTVVTADHSHVFTFGGYTPRGNSIFGLAPML
SDTDKKPFTAILYGNGPGYKVVGGERENVSMVDYA
HNNYQAQSAVPLRHETHGGEDVAVFSKGPMAHLLH
GVHEQNYVPHVMAYAACIGANLGHCAPASSAGSLA
AVECPPCPAPPVAGPSVFLFPPKPKDTLMISRTPE
VTCVVVDVSQEDPEVQFNWYVDGVEVHNAKTKPRE
EQFNSTYRVVSVLTVLHQDVVLNGKEYKCKVSNKG
LPSSIEKTISKAKGQPREPQVYTLPPSQEEMTKNQ
VSLTCLVKGFYPSDIAVEVVESNGQPENNYKTTPP
VLDSDGSFFLYSRLTVDKSRWQEGNVFSCSVMHEA
LHNHYTQKSLSLSLGKDDDDDDDDDD
asfotase alfa
SEQ ID NO: 6
LVPEKEKDPKYVVRDQAQETLKYALELQKLNTNVA
KNVIMFLGDGMGVSTVTAARILKGQLHHNPGEETR
LEMDKFPFVALSKTYNTNAQVPDSAGTATAYLCGV
KANEGTVGVSAATERSRCNTTQGNEVTSILRWAKD
AGKSVGIVTTTRVNHATPSAAYAHSADRDVVYSDN
EMPPEALSQGCKDIAYQLMHNIRDIDVIMGGGRKY
MYPKNKTDVEYESDEKARGTRLDGLDLVDTVVKSF
KPRYKHSHFIWNRTELLTLDPHNVDYLLGLFEPGD
MQYELNRNNVTDPSLSEMVVVAIQILRKNPKGFFL
LVEGGRIDHGHHEGKAKQALHEAVEMDRAIGQAGS
LTSSEDTLTVVTADHSHVFTFGGYTPRGNSIFGLA
PMLSDTDKKPFTAILYGNGPGYKVVGGERENVSMV
DYAHNNYQAQSAVPLRHETHGGEDVAVFSKGPMAH
LLHGVHEQNYVPHVMAYAACIGANLGHCAPASSLK
DKTHTCPPCPAPELLGGPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVYTLPPSREEMTK
NQVSLTCLVKGFYPSDIAVEWESNGQPENNYKTTP
PVLDSDGSFFLYSKLTVDKSRWQQGNVFSCSVMHE
ALHNHYTQKSLSLSPGKDIDDDDDDDDDD

### Total Sialic Acid Content

As described herein, TSAC may impact the half-life of the recombinant alkaline phosphatase in physiological conditions. Thus, the TSAC level may serve as a quality attribute for recombinantly-produced alkaline phosphatases such as, e.g., ALP201. Control of the TSAC range during manufacturing of the polypeptide can improve reproducibility batch to batch and can reduce heterogeneity in the produced polypeptides. In some embodiments, the TSAC is about 0.8 mol/mol to about 8.0 mol/mol of the recombinant alkaline phosphatase. In some embodiments, the TSAC is about 0.9 mol/mol to about 7.0 mol/mol of the recombinant alkaline phosphatase. In some embodiments, the TSAC is about 1.0 mol/mol to about 6.0 mol/mol of the recombinant alkaline phosphatase. In some embodiments, the TSAC is about 1.2 mol/mol to about 6.0 mol/mol of the recombinant alkaline phosphatase. In some embodiments, the TSAC is about 1.5 mol/mol to about 6.0 mol/mol of the recombinant alkaline phosphatase. In some embodiments, the TSAC is about 2.0 mol/mol to about 6.0 mol/mol of the recombinant alkaline phosphatase. In some embodiments, the TSAC is about 3.2 mol/mol to about 5.9 mol/mol of the recombinant alkaline phosphatase. In some embodiments, the TSAC is about 0.9 mol/mol, about 1.0 mol/mol, about 1.1 mol/mol, about 1.2 mol/mol, about 1.3 mol/mol, about 1.4 mol/mol, about 1.5 mol/mol, about 1.6 mol/mol, about 1.7 mol/mol, about 1.8 mol/mol, about 1.9 mol/mol, about 2.0 mol/mol, about 2.1 mol/mol, about 2.2 mol/mol, about 2.3 mol/mol, about 2.4 mol/mol, about 2.5 mol/mol, about 2.6 mol/mol, about 2.7 mol/mol, about 2.8 mol/mol, about 2.9 mol/mol, about 3.0 mol/mol, about 3.1 mol/mol, about 3.2 mol/mol, about 3.3 mol/mol, about 3.4 mol/mol, about 3.5 mol/mol, about 3.6 mol/mol, about 3.7 mol/mol, about 3.8 mol/mol, about 3.9 mol/mol, about 4.0 mol/mol, about 4.1 mol/mol, about 4.2 mol/mol, about 4.3 mol/mol, about 4.4 mol/mol, about 4.5 mol/mol, about 4.6 mol/mol, about 4.7 mol/mol, about 4.8 mol/mol, about 4.9 mol/mol, about 5.0 mol/mol, about 5.1 mol/mol, about 5.2 mol/mol, about 5.3 mol/mol, about 5.4 mol/mol, about 5.5 mol/mol, about 5.6 mol/mol, about 5.7 mol/mol, about 5.8 mol/mol, about 5.9 mol/mol, or about 6.0 mol/mol of the recombinant alkaline phosphatase. In some embodiments, the TSAC is about 3.2 mol/mol of the recombinant alkaline phosphatase. In some embodiments, the TSAC is about 5.0 mol/mol of the recombinant alkaline phosphatase. In some embodiments, the TSAC is about 5.9 mol/mol of the recombinant alkaline phosphatase.

### Pharmaceutical Compositions

A polypeptide described herein (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or SEQ ID NO: 5)) can be formulated as a pharmaceutical composition by a variety of methods known in the art.

The composition may include one or more, or all, of phosphate, proline, and sucrose. The composition may include, for example, phosphate and sucrose. For example, the composition may include phosphate (e.g., sodium phosphate), at a concentration of, e.g., from about 1 mM to about 100 mM, or from about 5 mM to about 20 mM phosphate, e.g., about 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM, or, e.g., about 10 mM. The composition may further include proline and/or sucrose. The composition may further include proline. The composition may further include sucrose. For example, the composition may include from about 1 mM to about 500 mM proline, e.g., from about 70 mM to about 280 mM, e.g., from about 50 mM to about 200 mM, e.g., about 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 300 mM, 400 mM, or 500 mM, e.g., about 140 mM proline and/or from about 1 mM to about 500 mM sucrose, e.g., from about 70 mM to about 280 mM, e.g., from about 50 mM to about 200 mM, e.g., about 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 350 mM, 400 mM, 450 mM, or 500 mM, e.g., about 140 mM sucrose or about 210 mM sucrose. In some embodiments, the composition includes a molar ratio of proline: sucrose of about 1:0 to about 1:3, e.g., about 1:1. In some embodiments, the composition includes about 140 mM proline. In some embodiments, the formulation includes about 140 mM sucrose. In some embodiments, the formulation includes about 210 mM sucrose. In some embodiments, the formulation includes about 210 mM sucrose and does not include proline. In some embodiments, the composition includes about 210 mM sucrose and about 10 mM phosphate (e.g., sodium phosphate). The formulation may further include from about 0.01% to about 0.5%, e.g., from about 0.01% to about 0.1%, e.g., about 0.05% polyoxyethylene (20) sorbitan monooleate (e.g., polysorbate 80 (PS80)). In some embodiments, the composition is formulated at a pH of about pH 7.0 to about pH 7.6 (e.g., about pH 7.1, 7.2, 7.3, 7.4, 7.5, or 7.6, e.g., about 7.3). In some embodiments, the composition includes about 10 mM phosphate, about 140 mM proline, about 140 mM sucrose, and about 0.05% polyoxyethylene (20) sorbitan monooleate (e.g., PS80) at a pH of about 7.3.

The composition may be formulated as a solution containing the polypeptide in an amount of, e.g., about 0.1 mg/mL to about 200 mg/mL, such as about 100 mg/mL. The composition may be formulated for intravenous or subcutaneous administration, e.g., at a dosage of the polypeptide of from about 0.1 mg/mL to about 10 mg/mL. The composition may be formulated in a solution in a volume of about 0.1 mL to about 50 mL (e.g., about 0.1 to about 10 mL, e.g., about 0.1 mL, 0.2 mL, 0.3 mL, 0.4 mL, 0.5 mL, 0.6 mL, 0.7 mL, 0.8 mL, 0.9 mL, or 1.0 mL, e.g., about 1 mL to about 10 mL, e.g., about 2 mL, 3 mL, 4 mL, 5 mL, 6 mL, 7 mL, 8 mL, 9 mL, or 10 mL). In some embodiments, the composition is formulated in about 1 mL.

The disclosure also features a vial containing a pharmaceutical composition as described herein. The vial may contain a solution in a volume of, e.g., from about 0.1 mL to about 10 mL (e.g., about 1 mL). The vial may contain the polypeptide in an amount of, e.g., about 0.1 mg/mL to about 500 mg/mL, e.g., about 1 mg/mL to about 200 mg/mL, e.g., about 50 mg/mL to about 150 mg/mL, e.g., about 100 mg/mL, of the polypeptide (e.g., a polypeptide of SEQ ID NO: 5 or a variant having at least 85% sequence identity thereto).

For example, the vial may contain a volume of about 0.25 mL, about 0.5 mL, about 0.75 mL, or about 1.0 mL of a solution containing the polypeptide of SEQ ID NO: 5 at a concentration of about 50 to about 100 mg/mL, in which the solution contains about 10 mM phosphate, about 140 mM proline, about 140 mM sucrose, and about 0.05% polyoxyethylene (20) sorbitan monooleate (e.g., PS80) at a pH of about 7.3.

### Formulations

The compositions including sALPs and sALP fusion polypeptides (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or SEQ ID NO: 5)) can be formulated according to standard methods. For instance, the sALP composition can be formulated, for example, as a buffered solution at a suitable concentration and suitable for storage at 2-8° C. (e.g., 4° C.). The sALP composition can also be formulated for storage at a temperature below 0°° C. (e.g., −20° C. or −80° C.). The sALP composition can further be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, the compositions described herein can be formulated to be stable in storage for at least 1 year at 2-8° C. (e.g., 4° C.). A composition can be formulated in a suitable volume, e.g., a volume of about 0.1 mL to about 10 mL.

The compositions including sALPs and sALP fusion polypeptides (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be in liquid form.

For example, compositions intended for systemic or local delivery can be in the form of injectable or infusible solutions. Accordingly, the sALP composition (e.g., a composition containing a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be formulated for administration by a parenteral mode (e.g., subcutaneous, intravenous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, subcutaneous, intradermal, intravenous, intranasal, intraocular, pulmonary, intramuscular, intra-arterial, intrathecal, intracapsular, intraorbital, intracardiac, intradermal, intrapulmonary, intraperitoneal, transtracheal, subcuticular, intraarticular, subcapsular, subarachnoid, intraspinal, epidural, intracerebral, intracranial, intracarotid, and intrasternal injection and infusion. Particular routes of administration include intravenous and subcutaneous administration.

The composition can be prepared as a lyophilized composition. The composition can be rehydrated with a solution (e.g., as described herein) prior to administration.

### Dosage

The sALP polypeptide (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1)) described herein can be administered to a subject having or being prone to a bone mineralization disorder, such as HPP, in individual doses ranging, e.g., from 0.01 mg/kg to 500 mg/kg (e.g., from 0.05 mg/kg to 500 mg/kg, from 0.1 mg/kg to 60 mg/kg, from 0.1 mg/kg to 50 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, 0.5 mg/kg to 25 mg/kg, 1.0 mg/kg to 10 mg/kg, 1.5 mg/kg to 5 mg/kg, or 2.0 mg/kg to 3.0 mg/kg) or from 1 μg/kg to 1,000 μg/kg (e.g., from 5 μg/kg to 1,000 μg/kg, from 1 μg/kg to 750 μg/kg, from 5 μg/kg to 750 μg/kg, from 10 μg/kg to 750 μg/kg, from 1 μg/kg to 500 μg/kg, from 5 μg/kg to 500 μg/kg, from 10 μg/kg to 500 μg/kg, from 1 μg/kg to 100 μg/kg, from 5 μg/kg to 100 μg/kg, from 10 μg/kg to 100 μg/kg, from 1 μg/kg to 50 μg/kg, from 5 μg/kg to 50 μg/kg, or from 10 μg/kg to 50 g/kg).

Exemplary doses of a sALP include, e.g., 0.01, 0.05, 0.1, 0.5, 1, 2, 2.5, 5, 10, 20, 25, 50, 100, 125, 150, 200, 250, or 500 mg/kg; or 1, 2, 2.5, 5, 10, 20, 25, 50, 100, 125, 150, 200, 250, 500, 750, 900, or 1,000 μg/kg. For all dosages or ranges recited herein, the term “about” can be used to modify these dosages by ±10% of the recited values or range endpoints. In particular, compositions (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) in accordance with the present disclosure can be administered to a subject in doses ranging from about 0.001 mg/kg/day to about 500 mg/kg/day, about 0.01 mg/kg/day to about 100 mg/kg/day, or about 0.01 mg/kg/day to about 20 mg/kg/day. For example, the sALP compositions (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be administered to a subject in a weekly dosage ranging, e.g., from about 0.5 mg/kg/week to about 140 mg/kg/week, e.g., about 0.8 mg/kg/week to about 50 mg/kg/week, or about 1 mg/kg/week to about 10 mg/kg/week (e.g., about 6 or about 9 mg/kg/week). In particular, the sALP can be administered one or more times per week (e.g., 1, 2, 3, 4, 5, 6, 7, or more times per week), one or more times every other week, or one or more times per month (e.g., once every 14 days, 15 days 16 days, 17 days, 18 days, 19 days, 20 days, 21 days, 22 days, 23 days, 24days, 25 days, 26 days, 27 days, 28 days, 29 days, or 30 days). In some embodiments, the formulation is administered once per week. In some embodiments, the formulation is administered once every two weeks.

In particular, the sALP (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be administered at a dosage of 2 mg/kg three times a week (total dose 6 mg/kg/week), 1 mg/kg six times a week (total dose 6 mg/kg/week), 3 mg/kg three times a week (total dose 9 mg/kg/week), 0.5 mg/kg three times a week (total dose of 1.5 mg/kg/week), or 9.3 mg/kg three times a week (total dose 28 mg/kg/week). The dosage may be adapted by the clinician in accordance with conventional factors such as the extent of the disease and different parameters from the subject having or being prone to a bone mineralization disorder, such as HPP. Alternatively, about 0.1 mg/kg to about 20 mg/kg (e.g., about 0.1 mg/kg to about 9 mg/kg) can be administered one time per week or one time every two weeks. The sALP composition can also be administered 1-10 times (e.g., 1, 2, 3, 4, 5, 6, 7, 8, 9, and 10) per week or per two weeks at a dosage of 1-90 mg per dose (e.g., in a volume of 0.1 mL to 100 mL). In some embodiments, the sALP composition is administered once per week. In some embodiments, the sALP composition is administered once every two weeks.

A composition containing a sALP or a sALP fusion polypeptide (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be administered to a subject in either a single dosage regimen or a multiple dosage regimen. Doses can be administered, e.g., hourly, bi-hourly, daily, bi-daily, twice a week, three times a week, four times a week, five times a week, six times a week, weekly, biweekly, monthly, bimonthly, or yearly. Alternatively, doses can be administered, e.g., twice, three times, four times, five times, six times, seven times, eight times, nine times, ten times, eleven times, or twelve times per day, week, or month. In particular, the dosing regimen is once, twice, or thrice weekly. The duration of the dosing regimen can be, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s), week(s), or month(s), or even for the remaining lifespan of the subject having or being prone to a bone mineralization disorder, such as HPP. The amount, frequency, and duration of dosage can be adapted by the clinician in accordance with conventional factors such as the extent of the disease and different parameters from the subject having or being prone to a bone mineralization disorder, such as HPP.

For example, the dosage of a sALP or sALP fusion polypeptide (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) may be from about 0.1 mg/kg of body weight to about 10 mg/kg of body weight administered subcutaneously or intravenously one or more (e.g., 2, 3, 4, 5, 6, or 7) times per week.

In some particular embodiments, the polypeptide (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) or a pharmaceutical composition containing the same may be administered at a dosage of from about 0.01 mg/kg to about 60 mg/kg (e.g., from about 0.1 mg/kg to about 50 mg/kg, e.g., from about 0.1 mg/kg to about 20 mg/kg, e.g., from about 0.1 mg/kg to about 10 mg/kg). The polypeptide may be administered once per day, week, month, or year (e.g., once per week). In some embodiments, the polypeptide is administered one or more times every 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The polypeptide may be administered at a dosage of from about 0.01 mg/kg/week to about 50 mg/kg/week (e.g., from about 0.01 mg/kg/week to about 40 mg/kg/week, e.g., from about 0.1 mg/kg/week to about 20 mg/kg/week, e.g., from about 0.1 mg/kg/week to about 10 mg/kg/week). The polypeptide may be administered for at least one day, one week, one month, one year, or longer (e.g., for 1-5 years or for the life of the subject).

In some embodiments, the pharmaceutical composition is administered subcutaneously. For example, from about 10 mg to about 100 mg (e.g., about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, e.g., about 15 mg, 45 mg, or 90 mg) may be administered to the subject subcutaneously, e.g., once or twice per week, e.g., for at least 1, 2, 3, 4, 5, 6, 7, 8, 9, 10, or more weeks.

In some embodiments, the pharmaceutical composition is administered intravenously. For example, from about 10 mg to about 100 mg (e.g., about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, e.g., about 15 mg, 45 mg, or 90 mg) may be administered to the subject intravenously, e.g., once or twice per week, e.g., for at least 1, 2, 3, 4, 5, 6, 7, 8, 9, 10, or more weeks.

In some embodiments, the pharmaceutical composition is administered intravenously and subcutaneously. In some embodiments, the pharmaceutical composition is administered intravenously and subcutaneously at the same time. In some embodiments, the composition is administered intravenously then subcutaneously. In some embodiments, the pharmaceutical composition is administered subcutaneously then intravenously. For example, the pharmaceutical composition may be administered intravenously to the subject in one or more doses (e.g., in a single dose), e.g., as a loading dose, and subsequently administered to the subject one or more times subcutaneously, e.g., as a maintenance dose (e.g., each dose administered about once per week for a duration of the therapy).

### Methods of Treatment

Provided herein are methods for treating or ameliorating at least one symptom of a subject with a bone mineralization disorder, such as HPP. Other diseases or disorders, such as bone fracture, osteoporosis, sclerosteosis, chondrocalcinosis, hypotonia, Duchenne's muscular dystrophy, tracheobronchomalacia, seizure, neurofibromatosis 1 (NF-1), and craniosynostosis may also be treated by the compositions and methods described herein. The subject may have muscle weakness. The subject may have a muscle weakness disease, such as calcium pyrophosphate deposition (CPPD) or familial hypophosphatemia. Such treatment may include administering an alkaline phosphatase (e.g., a pharmaceutical composition containing the alkaline phosphatase), or a polypeptide having alkaline phosphatase activity, to decrease the elevated PPi concentration in such subject. For example, a soluble alkaline phosphatase (sALP, e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) may be administered to neonates, infants, children, adolescents, or adults.

Subjects may be diagnosed with a bone mineralization disorder (e.g., HPP) prior to administration of an alkaline phosphatase or a polypeptide having alkaline phosphatase activity (e.g., a sALP, e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)). Additionally, a subject having or being prone to a bone mineralization disorder, such as HPP, can be a naïve subject that has not been previously treated with a sALP (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)).

One or more symptoms of the disease may first manifest in the subject as a neonate, infant, or child. In other embodiments, one or more symptoms of the disease may first manifest in the subject as an adult. In some embodiments, no detectable symptoms of the disease develop in the subject prior to adulthood. In some embodiments, detectable symptoms of the disease develop in the subject prior to adulthood, but the disease state remains undiagnosed until adulthood.

The method includes administering an alkaline phosphatase or a polypeptide having alkaline phosphatase activity (e.g., a sALP, e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) to a subject having or being prone to a bone mineralization disorder, such as HPP, in a single dose or in multiple dosages over a period of time. In particular, a sALP, such as a polypeptide having the sequence of SEQ ID NO: 5, can be administered to a subject previously determined to have elevated inorganic pyrophosphate (PPi) concentration or at least one predetermined biomarker/score for an HPP symptom (e.g., muscle weakness), such as an average BOT-2 strength score of less than 10, an average BOT-2 running speed and agility score of less than 5, an average CHAQ index score greater than about 0.8, and/or an average PODCI score of less than about 40, an average 6MWT of less than about 80% of the predicted 6MWT value, a Muscle Strength Grade of less than 5, and/or an average HHD value (e.g., an average HHD muscle or grip strength value) of, e.g., less than about 80% of the predicted HHD value. For example, a sALP can be administered to a subject previously determined to have a concentration of PPi in a sample (e.g., a plasma sample) of greater than about 5.71 μM for an infant or child (e.g., a subject of about 12 years of age or less); greater than about 4.78 μM for an adolescent (e.g., a subject of about 13 to about 18 years of age); or greater than about 5.82 μM for an adult (e.g., a subject of greater than about 18 years of age). In other embodiments, the bone mineralization disorder, such as HPP, described herein is caused by an elevated concentration of at least one alkaline phosphatase substrate (e.g., PPi, PLP, PEA, etc.). Alternatively, an alkaline phosphatase, or a polypeptide having alkaline phosphatase activity, (e.g., a sALP, e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be administered to a subject having or being prone to a bone mineralization disorder, such as HPP, prior to determination of muscle weakness score (e.g., using the BOT-2 strength score, BOT-2 running speed and agility score, the CHAQ index score, the BSID-III scaled score, the PDMS-2 standard score, a Muscle Strength score, a 6MWT value, and/or a HHD value). Treatment with an ALP according to the methods described herein promotes, e.g., an increase in activities of ADL, a decrease in pain, and/or an improvement in motor development.

Additionally, each of the described scores (e.g., the BOT-2 strength score, BOT-2 running speed and agility score, the CHAQ index score, the BSID-III scaled score, the PDMS-2 standard score, 6MWT, the 12-POMA-G, a modified performance-oriented mobility assessment (mPOMA-G, such as the one illustrated in Phillips et al. 2015 Bone Abstracts 4: P136), or the HHD value) of a subject having or being prone to a bone mineralization disorder, such as HPP, described herein can be used singly or in any combination to assess treatment efficacy using a sALP (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)), in which improvements relative to a certain test score demonstrate that the sALP is effective for treating the bone mineralization disorder, such as HPP.

For example, when administration of an alkaline phosphatase or a polypeptide having alkaline phosphatase activity (e.g., a sALP, e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) to a subject having or being prone to a bone mineralization disorder, such as HPP, results in an average increase in the BOT-2 strength score to about 10 or greater than about 10, in which the subject previously had an average BOT-2 strength score of less than about 10, then the alkaline phosphatase or a polypeptide having alkaline phosphatase activity treatment is effective at treating, e.g., physical impairments associated with a bone mineralization disorder, such as HPP. Alternatively, when administration of a sALP does not result in an average increase in the BOT-2 strength score to about 10 or greater than about 10, the dosage and/or frequency of alkaline phosphatase or a polypeptide having alkaline phosphatase activity administration can be changed (e.g., increased, for example, for an indefinite term or a short term (e.g., 1-6 months or up to one year or more) in order to determine the effective amount of the alkaline phosphatase or a polypeptide having alkaline phosphatase activity for the subject. For instance, the dosage of the sALP (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be increased from, e.g., from about 0.1-1 mg/kg/week to about 1-2 m/kg/week, from about 0.5-3 mg/kg/week to about 3-6 mg/kg/week or from about 3-6 mg/kg/week to about 6-9 mg/kg/week. Similarly, the frequency of dosage can be increased, e.g., from about once every three weeks to about once every two weeks or from about once every two weeks to about once every week. Alternatively, if improvement in one of the metrics described herein is achieved, the dosage and/or frequency of administration can remain the same or decrease from, e.g., about 6-9 mg/kg/week to about 3-6 mg/kg/week, from about 3-6 mg/kg/week to about 0.5-3 mg/kg/week, or from about 0.5-3 mg/kg/week to about 0.1-1 mg/kg/week. Similarly, the frequency of dosage administration can decrease from about once every week to about once every two weeks or from about once every two weeks to about once every three weeks.

Additionally, when administration of an alkaline phosphatase or a polypeptide having alkaline phosphatase activity (e.g., a sALP, e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) to a subject having or being prone to a bone mineralization disorder, such as HPP, results in an improvement in the Muscle Strength Grade categorization of the subject of one or more (e.g., an improvement to a Muscle Strength Grade of 1, 2, 3, 4, or 5 from a prior, lower Muscle Strength Grade), in which the subject previously had an average Muscle Strength Grade of less than about 5, then the alkaline phosphatase or a polypeptide having alkaline phosphatase activity treatment is effective at treating, e.g., physical impairments associated with a bone mineralization disorder, such as HPP. Alternatively, when administration of a sALP does not result in an improvement in the Muscle Strength Grade categorization of the subject of one or more from a prior, lower Muscle Strength Grade, the dosage and/or frequency of alkaline phosphatase or a polypeptide having alkaline phosphatase activity administration can be changed (e.g., increased) in order to determine the effective amount of the alkaline phosphatase or a polypeptide having alkaline phosphatase activity for the subject. For instance, the dosage of the sALP (e.g., a polypeptide having the sequence of SEQ ID NO: 5 or a variant thereof with at least 85% sequence identity thereto (e.g., a polypeptide that includes one or more, or all, of the mutations E108M, N213Q, and N286Q relative to SEQ ID NO: 1 or 5)) can be increased from, e.g., from about 0.1-1 mg/kg/week to about 1-2 m/kg/week, from about 0.5-3 mg/kg/week to about 3-6 mg/kg/week or from about 3-6 mg/kg/week to about 6-9 mg/kg/week. Similarly, the frequency of dosage can be increased, e.g., from about once every three weeks to about once every two weeks or from about once every two weeks to about once every week. Alternatively, if improvement in one of the metrics described herein is achieved, the dosage and/or frequency of administration can remain the same or decrease from, e.g., about 6-9 mg/kg/week to about 3-6 mg/kg/week, from about 3-6 mg/kg/week to about 0.5-3 mg/kg/week, or from about 0.5-3 mg/kg/week to about 0.1-1 mg/kg/week. Similarly, the frequency of dosage administration can decrease from about once every week to about once every two weeks or from about once every two weeks to about once every three weeks.

### Pharmacokinetic (PK) Parameters

In some embodiments, the treatment method produces an AUC₀₋₁₆₈ₕ of from about 50 μg×hour/mL to about 4000 μg×hour/mL in the blood of the subject. For example, the method may produce an AUC₀₋₁₆₈ₕ of from about 1000 μg×hour/mL to about 3000 μg×hour/mL in the blood of the subject. In some embodiments, the treatment method produces a Cₘₐₓ of from about 0.5 μg/mL to about 25 μg/mL in the blood of the subject. For example, the treatment method may produce a Cₘₐₓ of from about 0.6 μg/mL to about 20 μg/mL in the blood of the subject.

### EXAMPLES

The disclosure is illustrated by the following non-limiting examples. It is to be understood that the particular examples, materials, amounts, and procedures are to be interpreted broadly in accordance with the scope and spirit of the disclosure as set forth herein.

### Example 1

ALP201 is a human recombinant TNSALP-Fc-deca-aspartate fusion protein. It is a soluble glycoprotein composed of two polypeptide chains of 724 amino acids made from the catalytic domain of human TNSALP (SwissProt, P05186), the human immunoglobulin (Ig) G2/4 Fc domain (SwissProt, P01859, P01861) (to facilitate purification and extend half-life), and a deca-aspartate peptide (to target the bone).

ALP201 is an ERT for addressing the underlying cause of bone mineralization disorders, such as HPP by replacing a defective alkaline phosphatase enzyme. ALP201 shares some structural similarity to asfotase alfa, also a TNSALP-Fc-deca-aspartate fusion protein ERT, which is the only approved treatment for subjects with pediatric-onset HPP (marketed under the trade name STRENSIQ®).

ALP201 is a next-generation HPP therapy with equivalent potency and improved activity as compared to asfotase alfa. ALP201 also provides increased exposure due to a longer half-life, reduced a-phase clearance, and increased bioavailability. These improved characteristics support lower doses and longer dosing intervals for ALP201, relative to asfotase alfa. Subject experience is expected to improve by reducing injection volumes and dosing frequency, which may also translate to fewer injection site reactions.

### ALP201

### Physical and Chemical Characteristics

ALP201 is a soluble Fc fusion protein with a molecular weight of about 160 KDa and is composed of two polypeptide chains covalently linked by two disulfide bonds. Each polypeptide chain contains 724 amino acids and is composed of three segments:
The N-terminal region of the polypeptide, amino acids L1-A491, contains the enzyme TNSALP which is the soluble part of the human tissue non-specific alkaline phosphatase enzyme and contains the catalytic function. Within the enzymatic region of each polypeptide chain, a single point mutation (E108M) was introduced to improve enzyme activity and two N-linked glycosylation sites (N213Q and N286Q) removed for process improvements.
The second portion of the polypeptide, amino acids V492-K714, contains the Fc part of the human Immunoglobulin gamma 2/4 (lgG2/4) containing hinge, CH2 and CH3 domains.
The C-terminal region of the polypeptide, amino acids D715-D724, contains ten aspartic acids.

This peptide sequence promotes the binding of ALP201 to the mineral phase of bone. Each polypeptide chain of ALP201 contains four glycosylation sites (N123, N254, N413, and N564) and eleven cysteine (Cys) residues. Cys102 exists as a free cysteine. Each polypeptide chain contains four intra-chain disulfide bonds between Cys122 to Cys184, Cys472 to Cys480, Cys528 to Cys588, and Cys634-Cys692. The two polypeptide chains are connected by two inter-chain disulfide bonds between Cys494-Cys494 and Cys497-Cys497. In addition to these covalent structural features, mammalian alkaline phosphatases generally have four metal binding sites on each polypeptide chain, two sites for zinc, one site for magnesium and one site for calcium.

### General Properties of ALP201

Table 1 lists the general properties of ALP201. The theoretical chemical formula and theoretical average molecular weight were calculated assuming that all but two cysteine residues are disulfide bonded.

TABLE 1
General Properties of ALP201

| Characteristic | Result |
|---|---|
| Predicted Formula | C₇₀₅₈ H₁₀₉₂₈ N₁₉₅₂ |
| | O₂₁₉₈ S_{58 (aglycosylated)} |
| Predicted Molecular Weight | 160,154.6 Da (aglycosylated) |
| Number of Amino Acids | 1448 |
| Biologic Activity | Pyrophosphatase (PPi) and pyridoxal |
| | 5′-phosphate (PLP) hydrolysis, |
| | Hydroxyapatite binding. |

### Drug Product

### ALP201 100 mg/mL Vial

The ALP201 Drug Product (100 mg/mL) is a sterile, preservative-free formulated liquid solution of ALP201 and excipients contained in a single use 2 mL vial. The drug product does not contain any novel excipient or excipients of animal or human origin. Each 2 mL vial nominally contains 1.2 mL (overfill) of the drug product to deliver 100 mg of ALP201/vial. The drug product is filled into a 2 mL Type I clear glass vial with a 13 mm chlorobutyl stopper with aluminum seal. The quantitative composition of the ALP201 Drug Product is presented in Table 2.

TABLE 2
Composition of ALP201 Drug Product for
Subcutaneous or Intravenous Administration

| | Quantity | | | |
|---|---|---|---|---|
| Ingredient | per Vial | | Function | Standard |
| ALP201 | 100 | mg/mL | Active | In-house |
| | | | ingredient | standard |
| Sodium phosphate | 0.4 | mg/mL | Buffering | Multi-compendial |
| monobasic | | | Agent | |
| monohydrate | | | | |
| Sodium phosphate | 1.9 | mg/mL | Buffering | Multi-compendial |
| dibasic heptahydrate | | | Agent | |
| L-proline | 16.1 | mg/mL | Stabilizer | Multi-compendial |
| Sucrose | 47.9 | mg/mL | Tonicity | Multi-compendial |
| | | | modifier/ | |
| | | | stabilizer | |
| Polysorbate 80 | 0.5 | mg/mL | Adsorption | Multi-compendial |
| | | | Inhibitor | |

| | | | |
|---|---|---|---|
| Water for Injection | Q.S. | Aqueous | Multi-compendial |
| | | vehicle | |

Abbreviations:
QS = quantity sufficient.

### In Vivo Studies

ALP201 was evaluated using in vivo studies to characterize its pharmacology, PK/toxicokinetics (TK), local tolerability, and potential systemic toxicity. The pharmacology studies conducted include repeat dose studies in HPP mouse (Akp2GW (−/−) mouse), to identify the ALP201 minimal efficacious dose. To understand the PK properties of ALP201, single dose PK studies were conducted in mice, rat, and monkeys. The toxicology studies evaluated the systemic toxicity of the administration of SC ALP201 in rat and monkeys; these studies included safety pharmacology endpoints (cardiovascular, respiratory in monkey study and neurofunctional in rat study), TK evaluations, and a recovery period to assess the reversibility of any treatment-related effects.

A summary of the in vivo studies conducted with ALP201 that support its use in humans is provided in Table 3.

TABLE 3
Overview of ALP201 In Vivo Studies

| | | | | |
|---|---|---|---|---|
| | Test System/ | Method of | | |
| | Number of | Administration and | Study | |
| Study | animals | Dose (mg/kg) | Number | Results |

Pharmacology Studies

| | | | | |
|---|---|---|---|---|
| 36-Day Multi-Dose | Akp2GW(−/−) Mice | SC: 4.8 mg/kg on qd, | HPP-PoC-01 | ALP201 demonstrated efficacy in bone |
| Efficacy Study of | | q2d, and q1w dosing | | mineralization index and survival |
| ALP201 and Asfotase | | intervals | | endpoints at lower doses and |
| Alfa in HPP Mice | | SC: 4.8 mg/kg (days 1-24), | | longer dosing intervals than those used for asfotase |
| | | 1.5 mg/kg (days 25-35) | | alfa efficacious dose. |
| ALP201 Dose Titration | Akp2GW(−/−) Mice | SC: 0.15, 0.3, 0.8, or 2.0 | HPP-MED-01 | ALP201 demonstrated efficacy in bone |
| to Determine | | mg/kg/dose (q2d dosing) | | mineralization index, bone alkaline phosphatase |
| Mineralization ED85 in | | | | activity, survival, and end of study plasma level |
| Akp2GW −/− Mice | | | | endpoints at much lower doses than those used in |
| | | | | HPP-PoC-01. Dose response for bone mineralization |
| | | | | index observed for ALP201 across doses, with 2.0 |
| | | | | mg/kg q2d dose performing slightly better than |
| | | | | asfotase alfa at 2.5 mg/kg qd. |

Pharmacokinetics Studies

| | | | | |
|---|---|---|---|---|
| Assessment of Single | C57BL/6 Mice | IV and SC: | HPP-PK-01 | Terminal half-life after IV administration measured |
| Dose Pharmacokinetics | | 4 mg/kg | | 48 hours. Absolute bioavailability following SC |
| of ALP201 Following IV | | | | administration was high at 96%. PK parameters, |
| and SC Administration | | | | including systemic exposure of ALP201 by AUC, |
| in Male C57BL/6 Mice | | | | was significantly improved relative to asfotase alfa. |
| An Assessment of | Sprague-Dawley | IV bolus: 1, 3, 9, or 27 | 20205897 | Following IV or SC administration, the ALP201 |
| Pharmacokinetics of | Rats | mg/kg | | PK as measured by enzyme activity was slightly |
| ALP201 Following IV | 2M/2F per cohort | SC: 2 or 27 mg/kg | | less than proportional over the studied dose range |
| and SC Administration | | | | (1 to 27 mg/kg). Terminal half-life averaged about |
| in Rats | | | | 2 days. Absolute bioavailability following SC |
| | | | | administration was 61% and 54% for the 2 and |
| | | | | 27 mg/kg dose, respectively. |
| | | | | Single dose of ALP201 administered IV or SC |
| | | | | (from 1 to 27 mg/kg) was well tolerated in |
| | | | | male and female rats. |
| An Assessment of the | Cynomolgus | IV bolus: 2, 6, or 20 | 20205899 | Following IV or SC administration, the ALP201 |
| Pharmacokinetics of | Monkeys | mg/kg | | PK as measured by enzyme activity was close to |
| ALP201 Following IV | 3M per cohort | SC: 2 or 20 mg/kg | | dose-proportional over the studied dose range |
| and SC Administration | | | | (2 to 20 mg/kg). Terminal elimination half-life |
| in NHP | | | | averaged about 3 days. Absolute bioavailability |
| | | | | following SC administration was 69.6% and |
| | | | | 86.9% for the 2 and 20 mg/kg dose, respectively |
| | | | | Single dose of ALP201 administered IV or SC |
| | | | | (from 2 to 20 mg/kg) was well tolerated in male |
| | | | | nonhuman primates. |

Nonclinical Safety Studies (Toxicology and Safety-Pharmacology)

| | | | | | | |
|---|---|---|---|---|---|---|
| ALP201: A 28-Day | GLP | Sprague- | Repeat-dose | Charles | 1727-227 | No noteworthy systemic organ toxicity or local |
| Toxicity Study in Rats | | Dawley | SC: 0, 2, 10, or | River | | tolerability findings were observed at any of the |
| with a 28-Day Recovery | | Rats | 30 mg/kg/dose | Laboratories | | doses evaluated in the study. No noteworthy |
| Period | | | (q3d; 10 doses | | | neurotoxicity findings were observed at any |
| | | | total) | | | of the doses evaluated in the study. |
| | | | Single-Dose | | | Immunogenicity: (positive ADA) responses were |
| | | | IV: 0 or 10 | | | observed at all of the doses evaluated in the study. |
| | | | mg/kg/dose | | | The high dose of 30 mg/kg/dose evaluated in the |
| | | | (to evaluate SC | | | study is the NOAEL |
| | | | absolute | | | |
| | | | bioavailability) | | | |
| ALP201: A 28-Day | GLP | Cynomolgus | Repeat-dose | Charles | 1727-228 | No noteworthy systemic organ toxicity or local |
| Toxicity Study by | | Monkeys | SC: 0, 1, 5, or | River | | tolerability findings were observed at any of the |
| Subcutaneous Injection | | | 20 mg/kg/dose | Laboratories | | doses evaluated in the study. No noteworthy r |
| in Cynomolgus | | | (q3d; 10 doses | | | cardiovascula or respiratory findings were |
| Monkeys with a 28-Day | | | total) | | | observed at any of the doses evaluated in the study. |
| Recovery Period | | | | | | Immunogenicity: (positive ADA) responses were |
| | | | | | | observed at all of the doses evaluated in the study. |
| | | | | | | The high dose of 20 mg/kg/dose evaluated in the |
| | | | | | | study is the NOAEL |

Abbreviations:
F = female;
GLP = Good Laboratory Practice;
IV = intravenous;
M = male;
NOAEL = no observed adverse effect level;
qd = once a day;
q1w = once a week;
q2d = every other day;
SC = subcutaneous;
TBD = to be determined.

### In Vitro Pharmacology

Pyrophosphate (PPi) is a critical natural substrate of TNSALP, and low TNSALP levels in HPP subjects result in elevated circulating PPi blood levels. Elevated pyrophosphate levels prevent proper bone mineralization and lead to abnormal bone manifestations observed in subjects with HPP. Therefore, PPi was viewed as a natural substrate to target with higher enzyme activity levels in an engineered second-generation asfotase alfa molecule.

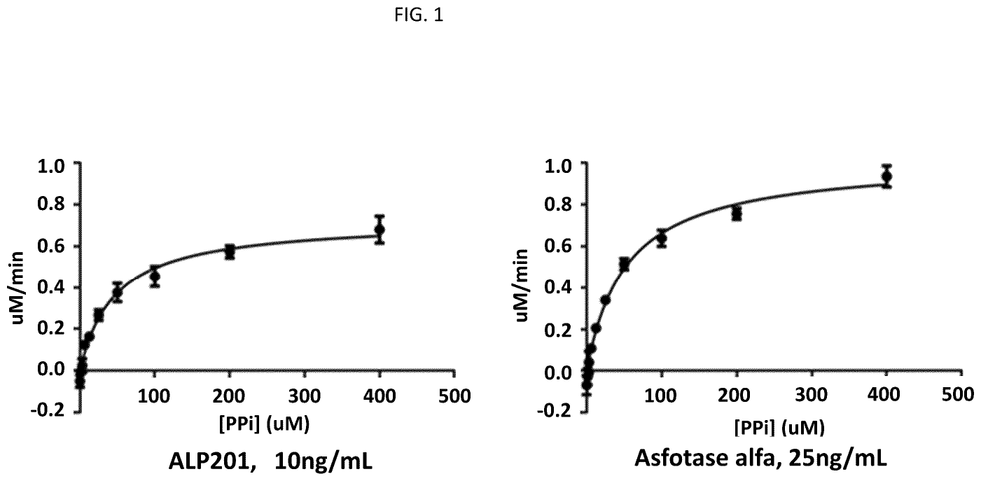

Substrate saturation curves for pyrophosphate hydrolysis by ALP201 and asfotase alfa are shown in FIG. 1. ALP201 retains a similar Km value (47 mM) for pyrophosphate as asfotase alfa (53 mM), but ALP201 operates with a significantly higher turnover number (kcat=11,619 min⁻¹ for ALP201 vs 6,714 min^{-31 1} for asfotase alfa). Similar Km values for ALP201 and the wildtype TNSALP catalytic domain in asfotase alfa suggest that ALP201 should not be more likely than asfotase alfa to dangerously lower circulating PPi levels when present at equivalent serum alkaline phosphatase activity levels.

Pyridoxyl-5′-phosphate is a second natural substrate of TNSALP with clinical relevance to HPP. TNSALP cleaves PLP to form pyridoxal, the B6 vitamer that is most easily taken in by tissues. When serum alkaline phosphatase levels are very low, systemic Vitamin B6 metabolism can be impaired. In HPP, PLP deficiency in the brain manifests as seizures, and systemic deficiencies in PLP hydrolysis by TNSALP may play a role in the pain, muscle weakness, and hypotonia experienced by some HPP subjects. Therefore, ERT for the treatment of HPP can be used to provide sufficient PLP hydrolysis activity.

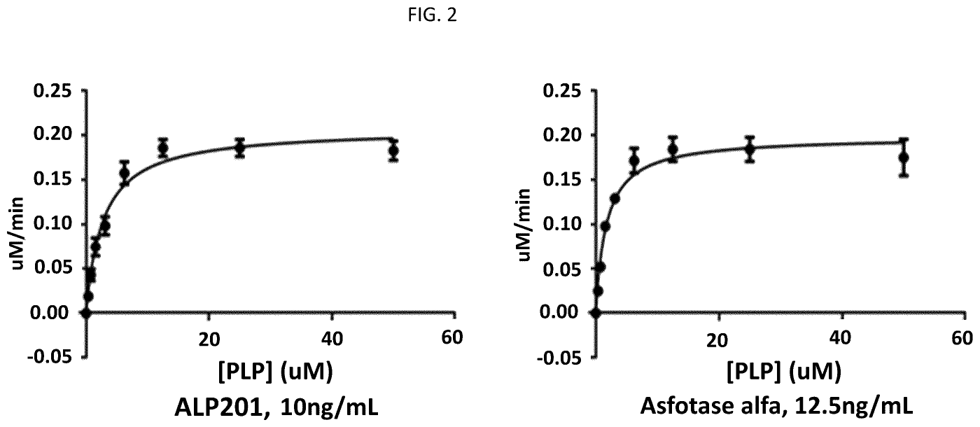

Substrate saturation curves for PLP hydrolysis by ALP201 and asfotase alfa are shown in FIG. 2. ALP201 has a slightly weaker Km value (2.76 mM) for PLP compared to asfotase alfa (1.71 mM), but ALP201 operates with a significantly higher turnover number (kcat=3,324 min⁻¹ for ALP201 vs 2,623 min⁻¹ for asfotase alfa), resulting in very similar PLP activity saturation curves for the two molecules. Substrate kinetic parameters are tabulated in Table 4.

TABLE 4
Kinetic parameters for substrate hydrolysis
by ALP201 and asfotase alfa

| Substrate | Construct | Km (μM) | Kcat (min⁻¹) |
|---|---|---|---|
| PPi | ALP201 | 46.8 +/− 5.7 | 11,619 +/− 449 |
| | asfotase alfa (Strensiq) | 53.2 +/− 5.5 | 6,714 +/− 226 |
| | STRENSIQ ® | | |
| PLP | ALP201 | 2.76 +/− 0.3 | 3,324 +/− 97 |
| | asfotase alfa (Strensiq) | 1.71 +/− 0.2 | 2,623 +/− 75 |
| | STRENSIQ ® | | |

Abbreviations:
PPi = pyrophosphate;
PLP = Pyridoxyl-5′-phosphate;
Km = Michaelis-Menten Constant:
μM = micromolar;
Kcat = catalytic constant for turnover rate;
min = minute.

### In Vivo Pharmacology

Preclinical efficacy studies on ALP201 were performed using the Akp2GW (-/-) mouse, which is an animal model of human HPP. The Akp2GW (−/−) mice share the same HPP-inducing TNSALP mutation used in the Akp2 (−/−) mice that were previously used in the preclinical evaluation of asfotase alfa. Natural history studies performed at Alexion during the development of the Akp2GW (−/−) mice showed that homozygotic TNSALP activity knockout Akp2GW (−/−) mice displayed a nearly identical bone mineralization and survival phenotype as Akp2 (−/−) mice (Akp2GW-NH-01-02).

In efficacy studies used to evaluate the efficacy of ALP201 using the Akp2GW (−/−) mouse model, doses of test articles were given subcutaneously, beginning on Day 1 after birth until Day 35. Outcomes reported in all studies included overall survival, body weight growth rate, bone mineralization of hind paw bones on Day 36 (or at death if before end-of-study [EOS]), and EOS trough plasma enzyme activity levels (taken on Day 36 in every day [qd] and every week [q1w] groups, and on Day 37 in every 2 days [q2d] dose groups). In some studies, EOS femur and tibia lengths and mouse femur alkaline phosphatase activity were determined.

In previous studies using Akp2 (−/−) mice, asfotase alfa demonstrated efficacy in both bone mineralization and overall survival study endpoints at a dose of 7-10 mg/kg/day for active preparations of asfotase alfa, depending upon the specific activity of the test article. The estimated minimum efficacious dose (MED) of asfotase alfa was defined as the dose that resulted in 85% of measurable mice in a group obtaining normal bone mineralization score by the end of the study or at death, if death occurred prior to the end of the study. An analysis of efficacy data placed this value at roughly 2.0-2.5 mg/kg/day, depending upon the specific activity of the test article.

The efficacy of ALP201 in a murine model of HPP was tested in 2 multi-dose studies at varying SC doses and dosing intervals in Akp2GW (−/−) mice. In these studies, efficacy of ALP201 was compared to efficacy observed in a positive control group dosed with daily SC administration of asfotase alfa.

In a previous study, asfotase alfa was dosed by SC administration at its fully efficacious dose of 9.8 mg/kg/day on a qd dosing schedule. An equivalent 4-MUP activity dose of ALP201 was dosed by SC administration in PBS on qd, q2d, and q1w dosing intervals. In one dose group, the dose of ALP201 was reduced by one-half log after weaning of the mice at Day 25. Subcutaneous administration of PBS on a qd schedule was used as a negative control. Tabulation of these dosing groups can be found in Table 5.

In the HPP-MED-01 study, asfotase alfa was dosed by SC administration at its minimum efficacious dose of 2.5 mg/kg/day on a qd dosing schedule. ALP201 was administered subcutaneously in PBS on a q2d dosing schedule at doses of 2.0, 0.8, 0.3, and 0.15 mg/kg. Subcutaneous administration of PBS on a q2d schedule was used as a negative control. Tabulation of these dosing groups can be found in Table 5.

TABLE 5
Dose-Grouped Study Plans for Akp2GW (−/−) Efficacy Studies

| | | | Dose | | |
|---|---|---|---|---|---|
| | | | (U MUP | | |
| | Test | Dose | activity/ | In- | Mouse |
| Study | Article | (mg/kg) | kg) | terval | Genotype |
| HPP-PoC-01 | ALP201 | 4.8 | 206 | qd | Akp2GW (−/−) |
| HPP-PoC-01 | ALP201 | 4.8 (days | 206 (days | qd | Akp2GW (−/−) |
| | | 1-24) | 1-24) | | |
| | | 1.5 (days | 62 (days | | |
| | | 25-35) | 25-35) | | |
| HPP-PoC-01 | ALP201 | 4.8 | 206 | q2d | Akp2GW (−/−) |
| HPP-PoC-01 | ALP201 | 4.8 | 206 | q1w | Akp2GW (−/−) |
| HPP-PoC-01 | asfotase | 9.8 | 206 | qd | Akp2GW (−/−) |
| | alfa | | | | |
| HPP-PoC-01 | PBS | NA | NA | qd | Akp2GW (−/−) |
| HPP-PoC-01 | PBS | NA | NA | qd | Akp2GW (+/+) |
| HPP-MED-01 | ALP201 | 2 | 78 | q2d | Akp2GW (−/−) |
| HPP-MED-01 | ALP201 | 0.8 | 31 | q2d | Akp2GW (−/−) |
| HPP-MED-01 | ALP201 | 0.3 | 11.6 | q2d | Akp2GW (−/−) |
| HPP-MED-01 | ALP201 | 0.15 | 5.8 | q2d | Akp2GW (−/−) |
| HPP-MED-01 | asfotase | 2.5 | 54 | qd | Akp2GW (−/−) |
| | alfa | | | | |
| HPP-MED-01 | PBS | NA | NA | q2d | Akp2GW (−/−) |
| HPP-MED-01 | PBS | NA | NA | q2d | Akp2GW (+/+) |

Abbreviations:
PBS = phosphate buffer saline;
q1w = once weekly;
q2d = every 2 days;
qd = once daily;
U MUP = Units of activity in 4-methylumbelliferyl phosphate hydrolysis;
NA = not applicable.

### Bone Mineralization Outcomes

Day 36/37 bone mineralization outcomes were determined by X-ray analysis of the hind paws of treated Akp2GW (−/−) mice. X-ray visualization of hind paw bone mineralization was compared to Day 36 benchmark X-ray images exemplifying 4 classification categories: Unaffected, Slight Deficit, Moderate Deficit, and Severe Deficit. Detailed description of these classifications can be found in Table 6. Blinded individuals assigned a score to images for each mouse, and once complete, scores were compiled within individual dose groups.

TABLE 6
Classification for Day 36 Hind Paw Bone Mineralization Index Scoring

| | Classification of Bone | Description of Severity - | Description of Severity - |
|---|---|---|---|
| Score | Mineralization Deficit | Faxitron 2 D X-Ray | CT 3 D X-Ray |
| 1 | Severe | Profound dysmorphology and | Profound dysmorphology and |
| | | complete absence of medial | complete absence of either |
| | | and distal phalanges of the | proximal and/or medial and |
| | | digits and complete lack of | distal phalanges of the digits |
| | | secondary ossification centers | and complete lack of |
| | | | secondary ossification centers |
| | | | with variable metatarsal |
| | | | formation |
| 2 | Moderate | Fully formed digits, but still no | Fully formed digits with either |
| | | apparent secondary | no apparent secondary |
| | | ossification centers | ossification centers or variable |
| | | | emerging secondary center(s), |
| | | | variable sesamoids, and |
| | | | variable metatarsal formation |
| 3 | Slight | Fully formed digits with | Fully formed digits with either |
| | | variable but incomplete | missing secondary ossification |
| | | secondary ossification centers | center(s) or center(s) with |
| | | | variable development, |
| | | | misshapen and missing |
| | | | sesamoid(s) with variable |
| | | | metatarsal formation |
| 4 | Unaffected | Fully formed digits and all | Fully formed digits with all |
| | | secondary ossification centers | secondary ossification centers |
| | | present | present, all sesamoids present |
| | | | with variable morphology and |
| | | | fully formed metatarsals. |

The distribution of observed EOS hind paw bone mineralization index scores for each dosing group can be found in Table 7.

TABLE 7
End of Study Bone Mineralization Index Distribution of Hind
Paw Bones in Treated Akp2GW (−/−) Mice Groups

| | | | | # Slight | | # Moderate | # Severe |
|---|---|---|---|---|---|---|---|
| | # in | # Unaffected | | Deficit | | Deficit | Deficit |
| Dose Group | Group | (Score = 4) | | (Score = 3) | | (Score = 2) | (Score = 1) |
| WT PBS | 20 | 20 | (100%) | 0 | | 0 | 0 |
| ALP201 4.8 qd | 25 | 25 | (100%) | 0 | | 0 | 0 |
| ALP201 4.8/1.5 qd | 17 | 17 | (100%) | 0 | | 0 | 0 |
| ALP201 4.8 q2d | 24 | 24 | (100%) | 0 | | 0 | 0 |
| ALP201 2.0 q2d | 31 | 29 | (93.5%) | 2 | (6.5%) | 0 | 0 |
| ALP201 0.8 q2d | 29 | 18 | (62.1%) | 11 | (37.9%) | 0 | 0 |
| ALP201 0.3 q2d | 31 | 18 | (58.1%) | 10 | (32.3%) | 1 (3.2%) | 2 (6.5%) |
| ALP201 0.15 q2d | 29 | 14 | (48.3%) | 9 | (31.0%) | 6 (20.7%) | 0 |
| ALP201 4.8 q1w | 19 | 14 | (73.7%) | 2 | (10.5%) | 3 (15.8%) | 0 |
| asfotase alfa 9.8 qd | 16 | 16 | (100%) | 0 | | 0 | 0 |
| asfotase alfa 2.5 qd | 26 | 22 | (84.6%) | 4 | (15.4%) | 0 | 0 |
| HOM PBS (at death) | 50 | 7 | (14.0%) | 12 | (24.0%) | 24 (48.0%) | 7 (14.0%) |

Abbreviations:
HOM = homozygous knockout;
PBS = phosphate buffered saline;
qd = every day;
q2d = every 2 days;
q1w = once weekly;
WT = wildtype.

Treatment of Akp2GW (−/−) mice with ALP201 showed a dose response of increasing percentage of mice with unaffected hind paw bone mineralization with increasing ALP201 dose on the Q2D dosing interval. All ALP201 dose groups demonstrated statistically significant improvement relative to PBS-treated Akp2GW (−/−) controls (p<0.001 by one-way ANOVA analysis). These data were used to inform model-based analyses and predicted human dose projections.

### End-of-Study Trough Plasma Active Enzyme Concentration Outcomes

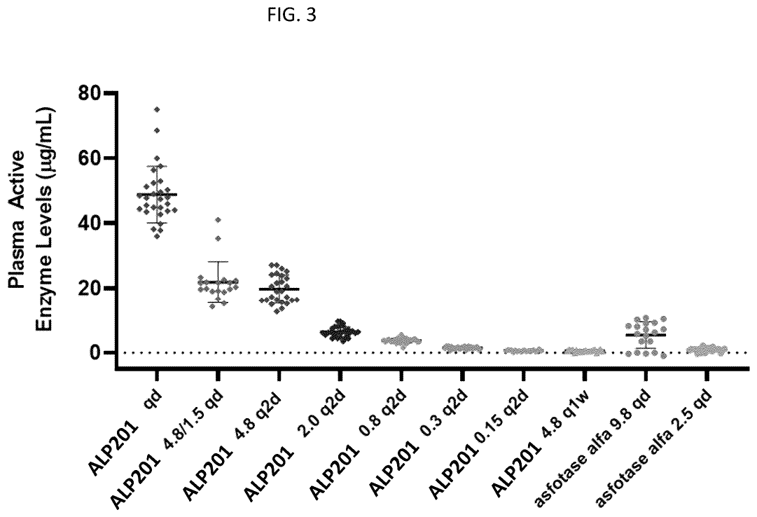

Trough plasma active TNSALP enzyme concentrations were determined by measurement of TNSALP activity levels in Day 36/37 plasma samples. Measured TNSALP activity was fit to a standard curve of known enzyme activity and concentration to quantify active enzyme levels per unit volume of plasma. The distribution of EOS trough plasma active enzyme concentration is shown in FIG. 3. A tabulation of the mean EOS trough plasma active enzyme concentration by dose group is summarized in Table 8.

TABLE 8
Dose Grouped Mean End-of-Study Trough Plasma Active
Enzyme Concentrations and Enzyme Activity Levels

| | | | | | Mean EOS | | |
|---|---|---|---|---|---|---|---|
| | | | | | Trough Plasma | Mean EOS | |
| | | | | Number | Activity Level | Enzyme | |
| | Test | Dose | | of Doses | (Units 4-MUP | Concentration | Day |
| Study | Article | (mg/kg) | Interval | in Study | Hydrolysis/L) | (μg/mL) | Collected |
| HPP-POC-01 | ALP201 | 4.8 | qd | 35 | 1805.2 ± 321.9 | 42.0^{a} | 36 |
| HPP-POC-01 | ALP201 | 4.8/1.5 | qd | 35 | 805.8 ± 235.0 | 18.7^{a} | 36 |
| HPP-POC-01 | ALP201 | 4.8 | q2d | 18 | 728.1 ± 162.4 | 16.9^{a} | 37 |
| HPP-MED-01 | ALP201 | 2 | q2d | 18 | 235.4 ± 58.7 | 6.1^{c} | 37 |
| HPP-MED-01 | ALP201 | 0.8 | q2d | 18 | 134.9 ± 27.6 | 3.5^{c} | 37 |
| HPP-MED-01 | ALP201 | 0.3 | q2d | 18 | 54.5 ± 14.9 | 1.4^{c} | 37 |
| HPP-MED-01 | ALP201 | 0.15 | q2d | 18 | 22.6 ± 9.4 | 0.6^{c} | 37 |
| HPP-POC-01 | ALP201 | 4.8 | qw | 5 | 21.2 ± 13.7 | 0.5^{a} | 36 |
| HPP-POC-01 | asfotase | 9.8 | qd | 35 | 129.5 ± 81.9 | 6.2^{b} | 36 |
| | alfa | | | | | | |
| HPP-MED-01 | asfotase | 2.5 | qd | 35 | 20.2 ± 13.9 | 0.9^{d} | 36 |
| | alfa | | | | | | |
| HPP-MED-01- | PBS-WT | NA | NA | NA | 1.7 ± 2.9 | NA | 36 |

Note:
Calculation of mean end-of-study enzyme concentration in (μg/mL) = Mean EOS Trough Plasma activity level (Units 4-MUP hydrolysis/L)/specific activity of protein (U/mg).
^{a}Specific activity of ALP201 used in study = 43 U/mg
^{b}Specific activity of asfotase alfa used in study = 21 U/mg
^{c}Specific activity in ALP201 used in study = 38.8 U/mg
^{d}Specific activity of asfotase alfa used in study = 21.5 U/mg
Abbreviations:
EOS = end-of-study;
4-MUP = 4-methylumbelliferyl phosphate;
NA = not applicable;
PBS = phosphate buffered saline;
qw = once weekly;
q2d = every 2 days;
qd = every day;
WT = wildtype.

Analysis of the EOS trough plasma active enzyme and corresponding enzyme activity levels in treated Akp2GW (−/−) mice after multiple doses clearly shows greater absolute accumulation and dose-normalized accumulation of ALP201 when compared to asfotase alfa, presumably owing to the superior PK profile of ALP201 following SC administration.

### Day 36/37 Treated Akp2GW (−/−) Mouse End of Study Bone Enzyme Activity Levels

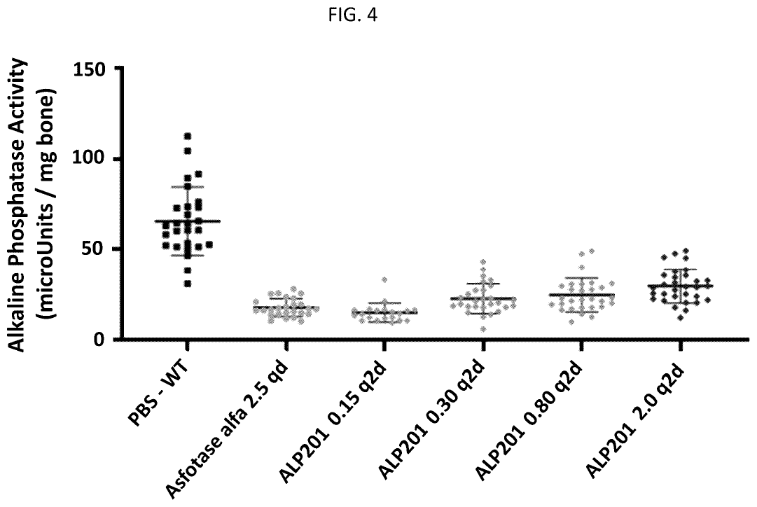

Alkaline phosphatase activity levels in treated Akp2GW (−/−) mouse femur tissue was determined in an ex vivo assay at the end of the HPP-MED-01 study, using 4-MUP as a substrate. In this assay, only the mineralized portion of the femur was assayed for activity to avoid potential contamination with blood or highly perfused tissue that might still hold high levels of ALP201 or asfotase alfa. The distribution of enzyme activity levels attached to treated Akp2GW (−/−) mouse femurs is shown in FIG. 4, with data displayed in microunits of 4-MUP hydrolysis activity/mg of mineralized femur tissue.

TABLE 9
Dose Grouped Mean End-of-Study Femur Active Enzyme Concentrations

| | | | | | Mean EOS Femur | |
|---|---|---|---|---|---|---|
| | | | | | Activity Level ± SD | |
| | | | | Number | (microUnits 4-MUP | |
| | | Dose | | of Doses | hydrolysis/mg | Day |
| Study | Test Article | (mg/kg) | Interval | in Study | bone) | Collected |
| HPP-MED-01 | ALP201 | 2 | q2d | 18 | 26.5 ± 9.4 | 37 |
| HPP-MED-01 | ALP201 | 0.8 | q2d | 18 | 20.8 ± 6.6 | 37 |
| HPP-MED-01 | ALP201 | 0.3 | q2d | 18 | 18.6 ± 6.5 | 37 |
| HPP-MED-01 | ALP201 | 0.15 | q2d | 18 | 13.0 ± 5.9 | 37 |
| HPP-MED-01 | asfotase alfa | 2.5 | qd | 35 | 14.8 ± 5.3 | 36 |
| HPP-MED-01 | PBS-WT | NA | NA | NA | 60.6 ± 19.6 | 37 |

Abbreviations:
4-MUP = 4-methylumbelliferyl phosphate;
PBS = phosphate buffered saline;
q1w = once weekly;
q2d = every 2 days;
qd = every day;
WT = wildtype;
mg = milligram;
SD = standard deviation;
NA = not applicable

Bone activity data showed a dose response with increasing EOS bone tissue activity levels with increasing ALP201 dose. At study end, mice in the ALP201 0.15 mg/kg q2d group had 22.6% of wildtype alkaline phosphatase activity restored, compared to 26.9% for the asfotase alfa 2.5 mg/kg qd group. ALP201 doses of 0.3, 0.8, and 2.0 mg/kg q2d restored 34.4%, 37.6%, and 44.9% of wildtype activity, respectively. End-of-study bone activity level differences between ALP201 doses at 0.8 and 2.0 mg/kg q2d were statistically significant from the asfotase alfa 2.5 mg/kg q2d group when analyzed with a one-way ANOVA with adjusted p values of 0.0048 and <0.0001, respectively.

### Treated Akp2GW (−/−) Mouse Survival Outcomes

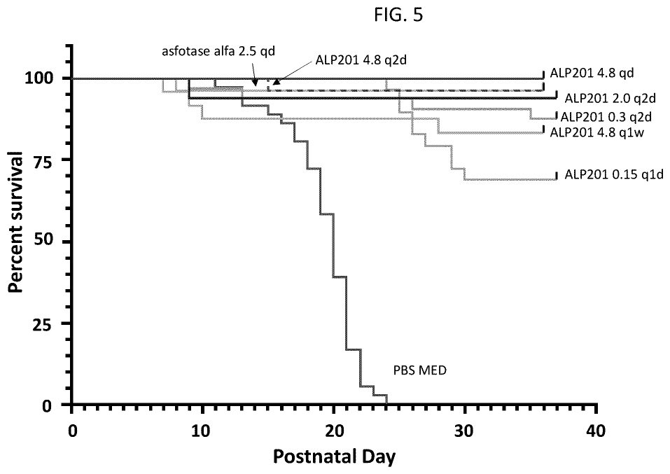

All PBS-treated Akp2GW (−/−) died on or before Day 26 of the studies, with a median survival time of 20 days. ALP201 treatment of Akp2GW (−/−) mice significantly improved 36-day survival rates in all dose groups relative to the PBS vehicle control (FIG. 5 and Table 10). All ALP201 dosing groups reached EOS survival rates of at least 69%, with all qd and q2d interval dose groups above a dose of 0.15 mg/kg/day posting 88% or greater overall survival rates. Survival curves for ALP201 4.8 mg/kg q1w group and asfotase alfa 9.8 mg/kg qd group have highly similar outcomes.

TABLE 10
Survival Percentage Summary of Akp2GW (−/−)
Mouse Groups Treated in 36-Day Efficacy Studies

| | | | | End of Study |
|---|---|---|---|---|
| Study | Test Article | Dose (mg/kg) | Interval | Survival (%) |
| HPP-PoC-01 | ALP201 | 4.8 | qd | 100 |
| HPP-PoC-01 | ALP201 | 4.8/1.5 | qd | 100 |
| HPP-PoC-01 | ALP201 | 4.8 | q2d | 96 |
| HPP-MED-01 | ALP201 | 2 | q2d | 94 |
| HPP-MED-01 | ALP201 | 0.8 | q2d | 97 |
| HPP-MED-01 | ALP201 | 0.3 | q2d | 88 |
| HPP-MED-01 | ALP201 | 0.15 | q2d | 69 |
| HPP-PoC-01 | ALP201 | 4.8 | q1w | 83 |
| HPP-PoC-01 | asfotase alfa | 9.8 | qd | 81 |
| HPP-MED-01 | asfotase alfa | 2.5 | qd | 96 |
| HPP-PoC-01 | PBS | NA | NA | 0 |
| HPP-MED-01 | PBS | NA | NA | 0 |

Abbreviation:
MED = minimum efficacious dose;
PBS = phosphate buffered saline;
PoC = proof of concept;
qd = every day;
q2d = every 2 days;
q1w = once weekly;
NA = not applicable.

### Body Weight Outcomes

Mean body weight of ALP201 and asfotase alfa treated Akp2GW (−/−) mice in all groups was consistently lower than that of their wild type littermates treated with PBS. End-of-study body weight for all ALP201 dosing groups was not statistically different from the asfotase alfa dosing groups.

### Safety Pharmacology

Standalone safety pharmacology studies were not performed for ALP201. However, cardiovascular, respiratory, and blood pressure endpoints were measured as part of the pivotal GLP-compliant monkey toxicity study and neurofunctional endpoints were assessed as part of the rat GLP toxicity study. ALP201 treatment resulted in increases in heart rates (28% compared with the control group) only at the low dose group, with associated decreases in the RR, PR, QT intervals and without any notable changes in QRS duration; no biologically meaningful changes were observed in the mid and high dose groups. Therefore, there was no dose-response relationship between slight increases in heart rate and ALP201 dose. Since the heart rate increases were modest in magnitude, these findings are not considered to be adverse. Additionally, there were no ALP201-related findings on ECG, blood pressure, or respiratory parameters evaluated in monkeys following once every 3 days SC administration for 28-days up to the highest dose, 20 mg/kg, evaluated in the study. ALP201 had no effects on behavioral indices or motor activity in rats following once every 3 days SC administration for 28-days up to the highest dose, 30 mg/kg, evaluated in the study.

### Pharmacokinetics and Drug Metabolism in Animals

Single dose PK studies were conducted in mice, rats, and monkeys. Data from these studies were used to assess the disposition (absorption, distribution, and elimination) of ALP201 in preclinical species.

### Absorption

The PK of ALP201 was assessed in the Sprague-Dawley rat and the Cynomolgus monkey. Summaries of the non-GLP and GLP studies following single- and repeat-dose administration of ALP201 are presented herein.

The mean bioavailability was 96%, 58%, and 78% for mouse, rat, and monkey, respectively. The time to reach maximum concentration (tₘₐₓ) ranged from 17 to 48 hours post-dosing, suggesting slow absorption of ALP201 from the SC injection site.

### Distribution

Following single intravenous administration in mouse, rat, and monkey, ALP201 volume of apparent distribution ranged from 0.08 to 0.15 L/kg. This value was much greater (>2-fold) than the plasma volume in these species, indicating ALP201 distribution beyond the intra-vascular compartment.

### Pharmacokinetics in Single-Dose Studies

### Single Dose PK in Male C57BL/6 Mice (Study HPP-PK-01)

The PK of ALP201 was evaluated following single dose IV or SC administration to male C57BL/6 mice. Sixteen animals received a single IV or SC administration of ALP201 at a dose of 4 mg/kg. For each administration route, mice were randomly subdivided into 4 sampling cohorts of 4 mice each. Blood samples were collected using a semi-serial sampling design for up to 20 days after dose administration. PK parameters of ALP201 are summarized in Table 11.

TABLE 11
PK Parameters of ALP201 Following IV and SC Administration in C57BL/6 Mice

| | | | | | | | V_{d} or | CL or | |
|---|---|---|---|---|---|---|---|---|---|
| | Dose | Cₘₐₓ | tₘₐₓ | t_{1/2} | AUCₜ | AUC_{∞} | V_{d}/F | CL/F | F |
| Route | (mg/kg) | (μg/mL) | (h) | (h) | (μg*h/mL) | (μg*h/mL) | (L/kg) | (L/h/kg) | (%) |
| IV | 4 | 93.3 | 1 | 48.1 | 2070.1 | 2114.3 | 0.13 | 0.0019 | NA |
| SC | 4 | 15.2 | 48 | 85.2 | 1828.7 | 2030.3 | 0.24 | 0.0020 | 96 |

Abbreviations:
AUC_{∞} = area under the concentration time curve from time zero (dosing) extrapolated to infinity;
AUCₜ = area under the concentration time curve from time zero (dosing) to the last detectable concentration;
CL or CL/F = total clearance;
Cₘₐₓ = maximum observed plasma concentration;
F = absolute bioavailability;
IV = intravenous;
NA = not applicable;
SC = subcutaneous;
tₘₐₓ = time to maximum observed plasma concentration;
t_{1/2} = terminal elimination half-life;
V_{d} or V_{d}/F = apparent volume of distribution

After IV administration, the concentration-time profile declined in a multi-exponential manner. The CL was 0.0019 L/h/kg and the apparent terminal t½ was 48 hours (Table 11

Tabl). The V_{d} was 0.13 L/kg, which is greater than plasma volume in mouse indicating ALP201 distribution beyond the intra-vascular space. After SC administration, the time to reach maximum concentration (tₘₐₓ) was 48 hours post dose, suggesting relatively slow absorption from the SC injection site. Absolute bioavailability following SC administration was 96%.

### Single-Dose PK Study in Rats

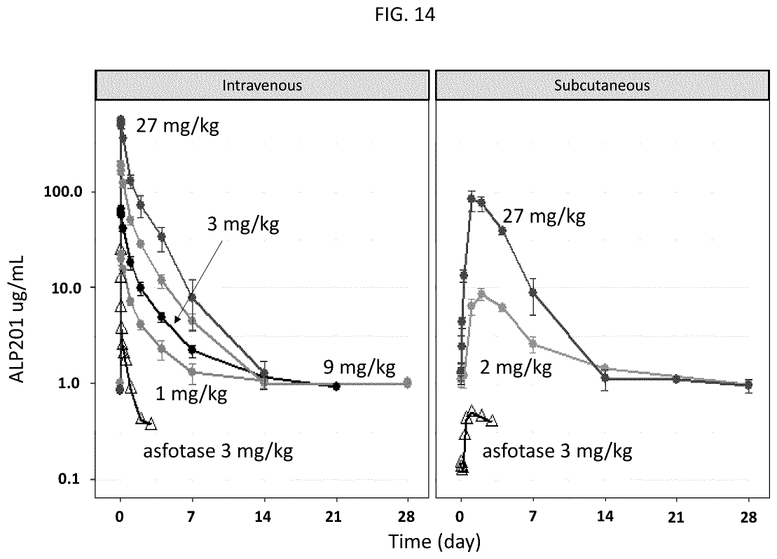

The PK of ALP201 was evaluated following single IV or SC administration to rats. Four animals (2 per sex/dose) received a single IV or SC ascending dose (IV bolus at a dose of 1, 3, 9, or 27 mg/kg or SC at a dose of 2 or 27 mg/kg) of ALP201. ALP201 plasma concentrations were quantifiable for up to 28 days post dose. As no apparent sex-specific differences in ALP201 PK parameters were observed, pooled sex values are provided. Descriptive statistics for PK parameters of ALP201 are summarized for the pooled sex groups in Table 12. ALP201 mean plasma concentration versus time profiles are shown in FIG. 14; note that historical data for asfotase alfa are added to enable comparison.

TABLE 12
Mean (±SD) PK parameters of ALP201 following IV or SC Administration in Rats

| | | | | | | | | V_{d} or | CL or |
|---|---|---|---|---|---|---|---|---|---|
| | Dose | | t_{1/2} | tₘₐₓ | Cₘₐₓ | AUCₜ | AUC_{∞} | V_{d}/F | CL/F |
| Route | (mg/kg) | N | (h) | (h) | (μg/mL) | (h × μg/mL) | (h × μg/mL) | (L/kg) | (L/h/kg) |
| IV | 1 | 4 | 66.6 ± | 0.5 ± | 22.4 ± | 716.5 ± | 802.1 ± | 0.12 ± | 0.0012 ± |
| | | | 5.6 | 0.0 | 0.8 | 47.3 | 60.1 | 0.02 | 0.0001 |
| IV | 3 | 4 | 58.7 ± | 0.6 ± | 65.7 ± | 1811.2 ± | 1934.4 ± | 0.13 ± | 0.0015 ± |
| | | | 14.0 | 0.3 | 1.5 | 142.3 | 196.0 (10.1) | 0.04 | 0.0002 |
| IV | 9 | 4 | 63.7 ± | 0.8 ± | 194.1 ± | 5299.7 ± | 5374.1 ± | 0.15 ± | 0.0017 ± |
| | | | 12.6 | 0.3 | 26.7 | 435.0 | 442.9 | 0.03 | 0.0001 |
| IV | 27 | 4 | 43.6 ± | 0.5 ± | 559.1 ± | 14089.0 ± | 14234.3 ± | 0.12 ± | 0.0019 ± |
| | | | 12.2 | 0.0 | 25.4 | 2003.9 | 2053.1 | 0.03 | 0.0002 |
| SC | 2 | 4 | 55.1 ± | 48.0 ± | 8.1 ± | 869.0 ± | 1007.7 ± | 0.16 ± | 0.0020 ± |
| | | | 6.7 | 0.0 | 1.9 | 123.4 | 124.6 | 0.03 | 0.0002 |
| SC | 27 | 4 | 38.3 ± | 30.4 ± | 82.2 ± | 7188.2 ± | 7674.2 ± | 0.19 ± | 0.0036 ± |
| | | | 6.9 | 11.7 | 19.6 | 1201.9 | 1406.1 | 0.03 | 0.0007 |

Abbreviations:
AUC_{∞} = area under the concentration time curve from time zero (dosing) extrapolated to infinity;
AUCₜ = area under the concentration time curve from time zero (dosing) to the last detectable concentration;
CL or CL/F = total clearance;
Cₘₐₓ = maximum observed plasma concentration;
IV = intravenous;
SC = subcutaneous;
SD = standard deviation;
tₘₐₓ = time to maximum observed plasma concentration;
t_{1/2} = terminal elimination half-life;
V_{d} or V_{d}/F = apparent volume of distribution.

For IV administration, ALP201 PK exposure (Cₘₐₓ and AUC_{∞}) increased in a slightly less than dose proportional manner over the studied dose range of 1 mg/kg to 27 mg/kg. The mean CL and V_{d} of ALP201 remained consistent across doses. The mean CL values ranged from 0.0012 to 0.0019 L/h/kg and mean V_{d} values ranged from 0.12 to 0.15 L/kg. Similarly, the t½ of ALP201, which remained relatively similar across IV doses, averaged about 2 days. For SC administration, the CL/F, V_{d}/F, and t_{1/2} values were similar to those estimated from IV administration, except for the 27 mg/kg SC dose group, which appeared to be an outlier. The mean tₘₐₓ ranged from 30 to 48 hours post dose, suggesting relatively slow absorption from the SC injection site. Absolute bioavailability following SC administration was 61% and 54% for the 2 and 27 mg/kg dose, respectively.

### Single-Dose PK Study in Monkeys

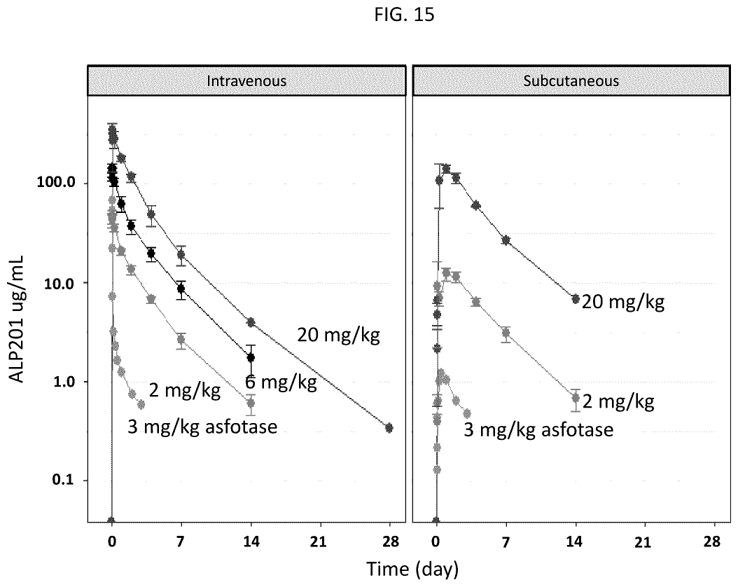

The PK of ALP201 was evaluated following single IV or SC administration to monkey. Three male monkeys received a single IV or SC ascending dose (IV bolus at a dose of 2, 6, or 20 mg/kg or SC at a dose of 2 or 20 mg/kg) of ALP201. ALP201 plasma concentrations were quantifiable for up to 28 days post dose. Descriptive statistics for PK parameters of ALP201 are summarized in Table 13Tabl. ALP201 mean plasma concentration versus time profiles are shown in FIG. 15. Note that historical data for asfotase alfa are added to enable comparison.

TABLE 13
Mean (±SD) PK parameters of ALP201 following
single IV or SC Administration in Monkeys

| | | | | | | | | V_{d} or | CL or |
|---|---|---|---|---|---|---|---|---|---|
| | Dose | | Tₘₐₓ | Cₘₐₓ | t_{1/2} | AUCₜ | AUC_{∞} | V_{d}/F | CL/F |
| Route | (mg/kg) | N | (h) | (μg/mL) | (h) | (h × μg/mL) | (h × μg/mL) | (L/kg) | (L/h/kg) |
| IV | 2 | 3 | 0.7 ± | 48.0 ± | 68.3 ± | 2298.4 ± | 2358.5 ± | 0.08 ± | 0.0008 ± |
| | | | 0.3 | 6.6 | 5.7 | 212.5 | 232.5 | 0.00 | 0.0001 |
| IV | 6 | 3 | 0.7 ± | 146.5 ± | 66.8 ± | 6394.2 ± | 6814.7 ± | 0.09 ± | 0.0009 ± |
| | | | 0.3 | 12.6 | 8.4 | 519.0 | 885.3 | 0.02 | 0.0001 |
| IV | 20 | 3 | 2.4 ± | 416.7 ± | 60.4 ± | 17838.9 ± | 18248.2 ± | 0.10 ± | 0.0011 ± |
| | | | 3.2 | 166.6 | 20.8 | 2737.0 | 2530.2 | 0.03 | 0.0002 |
| SC | 2 | 3 | 17.4 ± | 13.7 ± | 72.7 ± | 1593.4 ± | 1665.3 ± | 0.13 ± | 0.0012 ± |
| | | | 13.3 | 2.4 | 5.8 | 168.5 | 187.9 | 0.01 | 0.0001 |
| SC | 20 | 3 | 18.8 ± | 149.9 ± | 62.0 ± | 14064.2 ± | 15662.6 ± | 0.11 ± | 0.0013 ± |
| | | | 10.9 | 17.7 | 13.2 | 835.3 | 189.0 | 0.02 | 0.0 |

Abbreviations:
AUC_{∞} = area under the concentration time curve from time zero (dosing) extrapolated to infinity;
AUCₜ = area under the concentration time curve from time zero (dosing) to the last detectable concentration;
CL or CL/F = total clearance;
Cₘₐₓ = maximum observed plasma concentration;
CV = coefficient of variation;
IV = intravenous;
SC = subcutaneous;
SD = standard deviation;
tₘₐₓ = time to maximum observed plasma concentration;
t_{1/2} = terminal elimination half-life;
V_{d} or V_{d} = apparent volume of distribution.

For IV administration, the increases in Cₘₐₓ and AUC-values were close to dose proportional over the studied dose range of 2 mg/kg to 20 mg/kg. The CL and V_{d} of ALP201 remained consistent across doses, the mean CL values ranged from 0.0008 to 0.0011 L/h/kg and mean V_{d} values ranged from 0.08 to 0.10 L/kg. The t_{1/2} of ALP201, which remained relatively similar across doses as well, averaged about 3 days. For SC administration, the mean CL/F, V_{d}/F, and t_{1/2} values were consistent with those estimated after the IV administration. The mean tₘₐₓ ranged from 17 to 19 hours post-dose, suggesting relatively slow absorption from the SC injection site. Absolute bioavailability was 69.6% and 86.9% for the 2 mg/kg and the 20 mg/kg dose, respectively.

### Repeated Dose Toxicokinetics Studies with ALP201

Four-week SC Administration in Rats (1727-227): ALP201 was administered SC to rats at 2, 10 or 30 mg/kg/dose once every 3 days for 4 weeks. In addition, ALP201 was intravenously administrated to rats with single dose at 10 mg/kg to compare IV and SC route bioavailability. Pharmacokinetic parameters were calculated using non-compartment analysis from ALP201 composite plasma concentrations following Day 0 and Day 24 dosing and are shown in Table 14.

Systemic exposure to ALP201 appeared to be independent of sex following SC administration of ALP201 on Days 0 and 24 and following a single IV bolus injection of ALP201 on Day 0. Following SC administration of ALP201 every 3 days, Cₘₐₓ and AUC₀₋₇₂ₕ values for ALP201 increased with increasing dose in a slightly less than dose proportional manner on Days 0 and 24. Systemic exposure (AUC₀₋₇₂ₕ) to ALP201 appeared to decrease following repeated SC administration. The exposure reduction appeared to be dose dependent with maximum reduction up to 2-fold following repeated SC administration of 30 mg/kg ALP201. Subcutaneous bioavailability for ALP201 (based on AUC_{0-72 h} values at 10 mg/kg) was approximately 44.2%.

TABLE 14
Toxicokinetic Parameters following 4-Week Repeated SC or Single IV Administration in Rats

| | | | | Cₘₐₓ/ | | | AUC₀₋₇₂ₕ/ | | | |
|---|---|---|---|---|---|---|---|---|---|---|
| | | | | Dose | | | Dose | | | |
| | Dose | | Cₘₐₓ | (μg/mL)/ | Tₘₐₓ | AUC₀₋₇₂ₕ | (hr × μg/mL)/ | AUC₀₋₁₆₈ₕ | | F^{b} |
| Route | (mg/kg) | Day | (μg/mL) | (mg/kg) | (hr) | (hr × μg/mL) | (mg/kg) | (hr × μg/mL) | R^{a} | (%) |
| SC | 2 | 0 | 7.94 | 3.97 | 48 | 429 | 214 | 1320 | NA | NA |
| SC | 2 | 24 | 6.13 | 3.06 | 24 | 338 | 169 | 841 | 0.789 | NA |
| SC | 10 | 0 | 30.4 | 3.04 | 24 | 1630 | 163 | 4810 | NA | 44.2 |
| SC | 10 | 24 | 22.5 | 2.25 | 24 | 1200 | 120 | 2760 | 0.735 | NA |
| SC | 30 | 0 | 79.9 | 2.66 | 24 | 4150 | 138 | 11600 | NA | NA |
| SC | 30 | 24 | 43.9 | 1.46 | 24 | 2120 | 70.7 | 4380 | 0.512 | NA |
| IV | 10 | 0 | 195 | 19.5 | 2 | 3690 | 4720 | NA | NA | NA |

^{a}R = AUC₀₋₇₂ₕ Day 24/AUC₀₋₇₂ₕ Day 0.
^{b}F = AUC₀₋₇₂ₕ from 10 mg/kg SC dose group Day 0/AUC₀₋₇₂ₕ 10 mg/kg IV dose group Day 0 * 100
Abbreviations:
AUC₀₋₇₂ₕ = Area under the plasma concentration-time curve from 0 to 72 hours;
AUC₀₋₁₆₈ₕ = Area under the plasma concentration-time curve from 0 to 168 hours;
Cₘₐₓ = maximum observed plasma concentration;
IV = intravenous;
SC = subcutaneous;
NA = not applicable;
Tₘₐₓ = time to maximum observed plasma concentration.

Four-week SC Administration in Monkeys: ALP201 was SC administered to monkeys at 1, 5 or 20 mg/kg/dose once every 3 days for 4 weeks. The PK parameters were calculated using non-compartment analysis from ALP201 plasma concentrations following Day 1 and Day 24 dosing and are shown in Table 15.

TABLE 15
ALP201 Toxicokinetic Parameters following 4-Week Repeated SC Administration in Monkeys

| Dose | | Cₘₐₓ | Cₘₐₓ/Dose | Tₘₐₓ^{a} | AUC₀₋₇₂ₕ | AUC₀₋₇₂ₕ/Dose | AUC₀₋₁₆₈ₕ | |
|---|---|---|---|---|---|---|---|---|
| (mg/kg) | Day | (μg/mL) | (μg/mL)/(mg/kg) | (hr) | (hr × μg/mL) | (hr × μg/mL)/(mg/kg) | (hr × μg/mL) | R^{b} |
| 1 | 0 | 6.73 | 6.73 | 48 | 391 | 391 | 1440 | NA |
| 1 | 24 | 10.9 | 10.9 | NA | 787 | 787 | 2200 | 2.12 |
| 5 | 0 | 29.2 | 5.85 | 48 | 1720 | 345 | 6030 | NA |
| 5 | 24 | 38.1 | 7.62 | 24 | 2870 | 575 | 6970 | 1.83 |
| 20 | 0 | 125 | 6.25 | 48 | 7310 | 366 | 27900 | NA |
| 20 | 24 | 254 | 12.7 | 24 | 15400 | 772 | 36800 | 2.15 |

^{a}Median (minimum-maximum), median value only reported if actual collection interval
^{b}R = AUC₀₋₇₂ₕ Day 24/AUC₀₋₇₂ₕ Day 0.
Abbreviations:
AUC₀₋₇₂ₕ = Area under the plasma concentration-time curve from 0 to 72 hours;
AUC₀₋₁₆₈ₕ = Area under the plasma concentration-time curve from 0 to 168 hours;
Cₘₐₓ = maximum observed plasma concentration;
IV = intravenous;
NA = Not applicable;
SC = subcutaneous;
Tₘₐₓ = time to maximum observed plasma concentration.

Systemic exposure to ALP201 appeared to be independent of sex following SC administration of ALP201 on Days 0 and 24. Following subcutaneous administration of ALP201 every 3 days, Cₘₐₓ and AUC₀₋₇₂ₕ values for ALP201 increased with increasing dose in an approximately dose proportional manner on Days 0 and 24. Systemic exposure (AUC₀₋₇₂ₕ values) to ALP201 appeared to increase following repeated subcutaneous administration of ALP201 on Day 24.

### Pharmacokinetics, Pharmacodynamics, and Immunogenicity in Repeat-Dose Studies 28-Day Toxicity Study in Rats with a 28-Day Recovery Period

The TK/ADA of ALP201 was evaluated in male and female rats following SC administration of 2, 10, or 30 mg/kg once every three days (10/sex/dose group, 5/sex/timepoint) for 4 weeks (a total of 10 doses) and a single IV dose of 10 mg/kg (total of 1 dose). Blood samples for TK analysis were collected over a 72-hour period from all animals starting on Days 0 and 24. PK parameters of ALP201 are summarized in Table 16.

TABLE 16
Mean PK parameters of ALP201 on Day 0 and Day 24 Following Repeated SC
or Single IV Administration in Rats

| | Dose | | Cₘₐₓ | tₘₐₓ | AUC₇₂ₕ | AUC₁₆₈ₕ^{a} | | F^{c} |
|---|---|---|---|---|---|---|---|---|
| Route | (mg/kg) | Day | (μg/mL) | (hr) | (hr × μg/mL) | (hr × μg/mL) | R^{b} | (%) |
| SC | 2 | 0 | 7.94 | 48 | 429 | 1320 | NA | NA |
| SC | 2 | 24 | 6.13 | 24 | 338 | 841 | 0.789 | NA |
| SC | 10 | 0 | 30.4 | 24 | 1630 | 4810 | NA | 44.2 |
| SC | 10 | 24 | 22.5 | 24 | 1200 | 2760 | 0.735 | NA |
| SC | 30 | 0 | 79.9 | 24 | 4150 | 11600 | NA | NA |
| SC | 30 | 24 | 43.9 | 24 | 2120 | 4380 | 0.512 | NA |
| IV | 10 | 0 | 195 | 2 | 3690 | NA | NA | NA |

^{a}AUC₁₆₈ₕ values were calculated on day 0 and Day 18
^{b}R = AUC₇₂ₕ Day 24/AUC₇₂ₕ Day 0.
^{c}F = AUC₇₂ₕ from 10 mg/kg SC dose group Day 0/AUC₇₂ₕ 10 mg/kg IV dose group Day 0 * 100
Abbreviations:
AUC₇₂ₕ = area under the plasma concentration-time curve from 0 to 72 hours;
AUC₁₆₈ₕ = area under the plasma concentration-time curve from 0 to 168 hours;
Cₘₐₓ = maximum observed plasma concentration;
F = bioavailability;
IV = intravenous;
SC = subcutaneous;
NA = not applicable;
tₘₐₓ = time to maximum observed plasma concentration;
R = accumulation ratio

Systemic exposure to ALP201 appeared to be independent of sex following repeated SC administration of ALP201 and following a single IV bolus injection of ALP201. Therefore, pooled sex results were included Table 16. Following SC administration of ALP201 every 3 days (q3d), ALP201 exposure (Cₘₐₓ and AUC₇₂ₕ) increased with increasing dose in a slightly less than dose proportional manner on Days 0 and 24. Systemic exposure (Cₘₐₓ and AUC₇₂ₕ) on Day 24 appeared to decrease following repeated SC administration. The exposure reduction appeared to be dose dependent with maximum reduction up to 60% following repeated SC administration of 30 mg/kg ALP201. Subcutaneous bioavailability for ALP201 was approximately 44.2%.

The ADA response was negative for all pre-dose samples. The ADA incidence rate was 58% and 90% for D28 and D56 post-dose ADA samples, respectively. The exposure reduction on Day 24 (60%) for the 30 mg/kg dose was likely due to immunogenicity response.

### 28-Day Toxicity Study in Monkeys with a 28-Day Recovery Period

The TK/ADA of ALP201 was evaluated in male and female monkeys following SC administration of 1, 5, or 20 mg/kg once every three days (5/sex/dose group) for 4 weeks (a total of 10 doses). Blood samples for TK analysis were collected on Day 0 and 24. Descriptive statistics for PK parameters of ALP201 are summarized in Table 17.

TABLE 17
Mean (±SD) PK parameters of ALP201 on Day 0 and
Day 24 Following Repeated SC Administration in Monkeys

| Dose | | | Cₘₐₓ | tₘₐₓ^{a} | AUC₇₂ₕ | AUC₁₆₈ₕ^{b} | |
|---|---|---|---|---|---|---|---|
| (mg/kg) | Day | Statistic | (μg/mL) | (hr) | (hr * μg/mL) | (hr * μg/mL) | R^{c} |
| 1 | 0 | N | 10 | 10 | 10 | 10 | NA |
| | | Mean ± SD | 6.73 ± 1.44 | 48 (24-48) | 391 ± 99.9 | 1440 ± 298 | NA |
| 1 | 24 | N | 10 | 10 | 8 | 7 | 8 |
| | | Mean ± SD | 10.9 ± 7.48 | NA (8-24) | 787 ± 346 | 2200 ± 320 | 2.12 ± 1.11 |
| 5 | 0 | N | 10 | 10 | 10 | 10 | NA |
| | | Mean ± SD | 29.2 ± 4.44 | 48 (24-48) | 1720 ± 317 | 6030 ± 723 | NA |
| 5 | 24 | N | 10 | 10 | 8 | 8 | 8 |
| | | Mean ± SD | 38.1 ± 24.2 | 24 (8-24) | 2870 ± 1140 | 6970 ± 2600 | 1.83 ± 0.805 |
| 20 | 0 | N | 10 | 10 | 10 | 10 | NA |
| | | Mean ± SD | 125 ± 29.4 | 48 (24-48) | 7310 ± 1760 | 27900 ± 5130 | NA |
| 20 | 24 | N | 10 | 10 | 8 | 10 | 8 |
| | | Mean ± SD | 254 ± 62 | 24 (8-24) | 15400 ± 4420 | 36800 ± 12300 | 2.15 ± 0.59 |

^{a}Median (minimum-maximum), median value only reported if actual collection interval
^{b}AUC₀₋₁₆₈ₕᵣ values were calculated on Day 0 and Day 18
^{c}R = AUC₇₂ₕᵣ Day 24/AUC₇₂ₕᵣ Day 0.
Abbreviations:
AUC₇₂ₕ = Area under the plasma concentration-time curve from 0 to 72 hours;
AUC₁₆₈ₕ = Area under the plasma concentration-time curve from 0 to 168 hours;
Cₘₐₓ = maximum observed plasma concentration;
NA = Not applicable;
tₘₐₓ = time to maximum observed plasma concentration;
R = accumulation ratio.

There were no sex differences in the systemic exposure to ALP201 on Days 0 and 24. Therefore, pooled sex results were included in Table 17. Following subcutaneous administration of ALP201 once every 3 days, ALP201 (Cₘₐₓ and AUC₇₂ₕ) increased with increasing dose in an approximately dose proportional manner on Days 0 and 24. Systemic exposure (AUC₇₂ₕ values) to ALP201 appeared to increase following repeated subcutaneous administration of ALP201 on Day 24, with mean accumulation AUC₇₂ₕ ratios of 2.12, 1.83, and 2.15 at 1, 5, and 20 mg/kg, respectively.

The ADA response was negative for pre-dose samples. The ADA incidence rate was 23% and 92% for D28 and D56 post-dose ADA samples, respectively. Although a direct correlation could not be made between positive ADA responses and systemic exposure concentration-time profiles as ADA samples were not sampled on TK collection days, following multiple doses, aberrant concentration-time profiles for some of the animals at ≤5 mg/kg appeared to be impacted by anti ALP201 antibodies.

### Model-Based Analyses and Predicted Human Dose Regimens

### ALP201 Modelling

### Population Pharmacokinetics (Pop-PK) Model

To forecast ALP201 exposure in humans given mouse dose-response results, a Pop-PK model was developed using body weight-based allometric scaling and pooling mouse, rat, and monkey PK data to predict human PK parameter estimates. Mouse PK data included a single dose wild-type mouse study (HPP-PK-01) and single C_{trough} measurements (D36/D37) from 2 multiple dose Akp2GW (−/−) mouse efficacy studies (HPP-PoC-01 and HPP-MED-01). For rat and monkey, single dose, dose range-finding studies in rats and cynomolgus monkeys, and multiple dose 4-week GLP toxicology studies in rat and cynomolgus monkeys were also included.

The current Pop-PK model was developed using the NONMEM software program, version 7.2 (ICON solutions) to simultaneously analyze the IV and SC PK data from 3 animal species accounting for body-weight differences using allometric principles, where animal body weight was centered to 70 kg and allometric exponents were fixed to 0.75 for clearance parameters and 1.0 for volume of distribution parameters. The impact of ADA on PK was assessed on CL based on the 4-week TK showing that rats, and to a lesser degree, monkeys appeared to have lower ALP201 concentrations for ADA+animals. The current best Pop-PK model is a two-compartment model with linear elimination. The testing of ADA+ impact on ALP201 concentrations was inconclusive. The estimated bioavailability in humans was ˜75% and the calculated effective half-life for humans was 7 to 9 days. The human PK simulations used the estimated variability from the human asfotase alfa Pop-PK model and mean (and standard deviation) adult baseline body weights of HPP subjects who took part in the clinical trials evaluating asfotase alfa as a treatment for HPP.

### Dose-Response Model

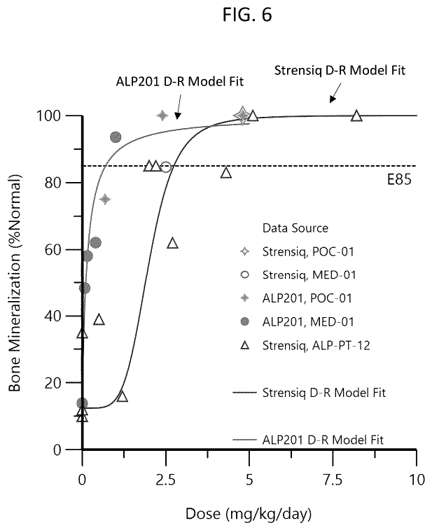

The developed dose-response model was selected by testing models from the Eₘₐₓ family. Most dose-response relationships can be described by one of the Eₘₐₓ model parameterizations. The current best dose-response characterization is using an Eₘₐₓ+E₀ (baseline) model. The efficacy endpoint, bone mineralization, was a radiographic assessment that was selected as it shared the same clinical definition of efficacy as that used for asfotase alfa nonclinical dose-response assessment. Bone mineralization after treatment with asfotase alfa or ALP201 from 2 efficacy studies (HPP-PoC-01 and HPP-MED-01) was plotted versus dose (FIG. 6). Asfotase alfa data from previous efficacy study was included for comparison. The y-axis was expressed as % Normal, which was defined as the percentage of mice in a dose group with bone mineralization scores of 4. The x-axis was expressed as dose normalized to mg/kg/day. The dose producing normal mineralization in 85% of the treated population (ED85) was chosen as the target effective dose.

### Dose Translation from Mouse to Human and Proposed Human Starting Doses

Allometric scaling was applied to mouse target effective dose (ED85) to determine a human dose. The equation used to predict from mouse ED85 to human equivalent dose (HED, mg/kg/day) was ED85×(0.025 kg mouse/70 kg human)^{0.25}. The HED was translated into a weekly flat dose of 45 mg/week and will be used as the dose for the FIH study Cohort 2 (both IV and SC). Cohort 3 (IV and SC) will use 90 mg/week. Cohort 1 will use a NOAEL-based starting dose of 15 mg/week (both IV and SC).

### Predicted Human Exposure and Safety PK Margins for ALP201

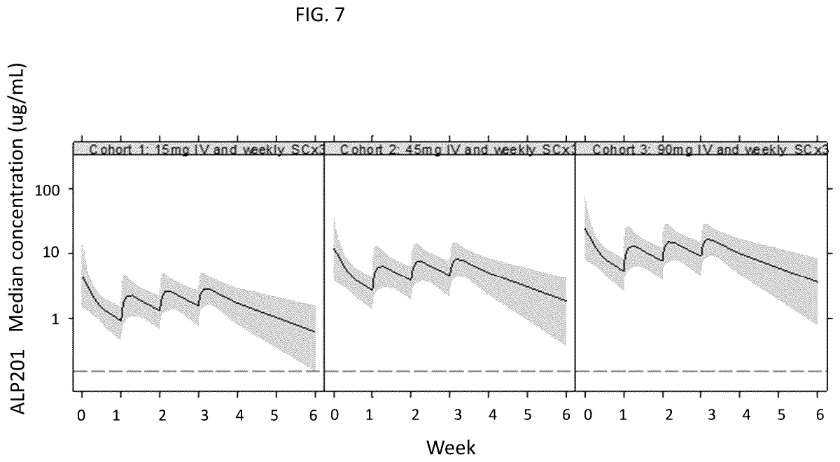

The Pop-PK model was used to simulate rich PK profiles based on allometrically-scaled doses to human (FIG. 7). Using the simulated PK data, the exposure metrics Cₘₐₓ and AUC were calculated.

The predicted human exposures (Cₘₐₓ and AUC₁₆₈ₕ) and expected safety PK margins for proposed FIH ALP201 doses are shown in Table 18. Safety PK margins were calculated for the proposed FIH ALP201 doses using predicted human exposure and observed NOAEL exposure from the 4-week GLP monkey toxicology study (NOAEL dose at 20 mg/kg/Q3D administered SC).

TABLE 18
Predicted ALP201 PK Exposure, Safety Margin,
and % Normalized Mineralization Response

| | | | | | | | % Normal |
|---|---|---|---|---|---|---|---|
| | | | Predicted | | Expected | Expected | Mineralization |
| | | | AUC₁₆₈ₕ | Predicted | Safety | Safety | Response in |
| | | | (μg × | Cₘₐₓ | Margin | Margin | Mouse HPP |
| Cohort | N | Dose | hour/mL) | (μg/mL) | for AUC₁₆₈ₕ* | for Cₘₐₓ* | Model |
| 1 | 4 | 15 mg IV | 325 | 4.3 | 113 | 59 | NA |
| | | 15 mg SC | 441 | 3.2 | 83 | 79 | 66 |
| | | biweekly × 3 | | | | | |
| 2 | 4 | 45 mg IV | 1084 | 13.0 | 38 | 20 | NA |
| | | 45 mg SC | 1318 | 9.6 | 28 | 26 | 85 |
| | | biweekly × 3 | | | | | |
| 3 | 4 | 90 mg IV | 1951 | 26 | 19 | 10 | NA |
| | | 90 mg SC | 2645 | 19 | 14 | 13 | 91 |
| | | biweekly × 3 | | | | | |

Abbreviations:
AUC₁₆₈ₕ = the area under the concentration time curve from time zero to 168 hour;
Cₘₐₓ = the maximum observed concentration measured after dosing;
HPP = hypophosphatasia;
IV = intravenous;
N = number of subjects;
SC = subcutaneous;
NA = not available
*NOAEL of 20 mg/kg/q3d from monkey 4-week SC toxicology study.

### Toxicology Studies

Nonclinical safety studies were conducted in rat and monkeys to evaluate local tolerability, systemic toxicity, and safety pharmacology parameters following SC administration of ALP201 for up to 28 days. Nonclinical safety evaluation of single-dose IV administration of ALP201 was also evaluated in rat 28-day toxicity study to characterize the bioavailability of SC ALP201.

### Single-Dose Toxicology Studies

Standalone single-dose tolerability studies were not conducted for ALP201. However, ALP201 tolerability in rats and monkeys was evaluated as part of single-dose PK studies. The tolerability of ALP201 was assessed by clinical observations, including site of injection reactions and clinical pathology data. No noteworthy ALP201-related injection site reactions, clinical observations, and clinical pathology observations were noted in rats or monkeys single-dose PK studies. In summary, single doses of ALP201 were well tolerated when administered via IV or SC in rats up to 27 mg/kg and in monkeys up to 20 mg/kg. The doses for ALP201 repeated dose definitive (GLP) toxicology studies were selected based on the tolerability and clinical pathology data obtained.

### Repeat Dose Toxicology Studies

Definitive or GLP-compliant 28-day toxicity studies were conducted in Sprague-Dawley rats and Cynomolgus monkeys. In rat 28-day toxicity study, administration of ALP201 via subcutaneous injection once every 3 days for 28 days (Days 1, 4, 7, 10, 13, 16, 19, 22, 25, and 28) for a total of 10 doses to male and female CD® rats was well tolerated up to 30 mg/kg/dose, the highest SC dose evaluated in this study. Additionally, administration of ALP201 at 10 mg/kg IV was also well tolerated after a single injection. There were no ALP201-related changes in clinical observations, body weights, body weight gains, quantitative food consumption, ophthalmology, functional observational battery, hematology, urinalysis, gross pathology, and organ weights. At the end of the dosing phase, there were dose-related increases in alkaline phosphatase (ALP) activity (anticipated pharmacology effect) at ≥2 mg/kg/dose ALP201 SC which were fully resolved following a 28-day recovery period. Non-adverse microscopic changes were related to the injection procedures with associated reactive changes in the draining lymph nodes. There was no discernible ALP201-associated alteration in the incidence, severity, or microscopic character of the changes at the injection sites or draining lymph nodes. Microscopic changes in the injection sites and draining lymph nodes of recovery animals were similar to those noted in the terminal necropsy animals, although less pronounced in the recovery group animals. In conclusion, the dose level of 30 mg/kg/dose is considered to be the no observed-adverse-effect-level (NOAEL) for subcutaneous administration; this corresponds to a Day 24 male and female combined Cₘₐₓ value of 43.9 g/mL and an AUC₀₋₇₂ hr value of 2120 hr*μg/mL.

Following a single IV bolus injection of 10 mg/kg ALP2-1 in rats resulted in an AUC₀₋₇₂ hr, of 3690 h*μg/mL. The SC bioavailability for ALP201 (based on SC AUC₀₋₇₂ hr values at 10 mg/kg/dose) in rats was approximately 44.2%.

In monkey 28-day toxicity study, administration of ALP201 via SC injection once every 3 days for 28 days (a total of 10 days on Days 1, 4, 7, 10, 13, 16, 19, 22, 25, and 28), at doses of 1 mg/kg, 5 mg/kg, or 20 mg/kg to male and female cynomolgus monkeys was well tolerated up to 20 mg/kg/dose, the highest dose evaluated in this study. There were no ALP201-related changes in injection site reactions (dermal scoring), body weights, body weight gains, qualitative food consumption, ophthalmology, manual respiratory rates, indirect blood pressures, qualitative electrocardiogramatology, urinalysis, gross pathology, and organ weights. At the end of the dosing phase, there were dose-related increases in alkaline phosphatase (ALP) activity (anticipated pharmacology effect), which were fully (1 mg/kg/dose) or mostly (>5 mg/kg/dose) resolved following a 28-day recovery period. Non-adverse ALP201-related microscopic changes were confined to the injection sites and included minimal to mild degeneration/necrosis, mineralization, and mixed cell inflammation/infiltration. Partial recovery of degeneration/necrosis and mixed cell inflammation/infiltration of the injection site(s) were observed within the recovery groups, while minimal to mild mineralization persisted in recovery males at ≥1 mg/kg/dose and females at 20 mg/kg/dose. Heart rate increases were observed in all animals at 1 mg/kg/dose and in some of the animals at >5 mg/kg/dose. These heart rate increases were not considered to be adverse because, in addition to systemic exposure data incongruity, the heart rate and ECG values remained within the normal range of biological variation for monkeys of this age. In conclusion, the dose level of 20 mg/kg/dose is considered to be the no observed-adverse-effect-level (NOAEL), this corresponds to a Day 24 male and female combined mean Cₘₐₓ value of 254 μg/mL and a mean AUC₀₋₇₂ hr value of 15400 hr*μg/mL respectively.

### Local Tolerance

Standalone nonclinical studies were not conducted to evaluate local tolerability. However, evaluation of injection sites was performed in the SC ALP201 in rat and monkey general toxicology studies and IV ALP201 in rat general toxicology study. Subcutaneous and IV administrations of ALP201was well tolerated locally in monkeys, with no adverse findings observed at the sites of injection

### Summary of Nonclinical Observations Relevant to Clinical Studies of ALP201

### Safety Assessment

There were no ALP201-related changes in clinical observations, body weights, body weight gains, quantitative food consumption, ophthalmology, functional observational battery (CNS) observations, cardiovascular endpoints, respiratory endpoints, hematology, urinalysis, gross pathology, and organ weights 28-day toxicology studies. At the end of the dosing phase, there were significant dose-related increases in alkaline phosphatase (ALP) activity, an anticipated pharmacology effect in rats or monkeys. No noteworthy systemic organ toxicities were observed in rats or monkeys at any of the doses evaluated in respective studies.

ALP201 was well tolerated locally following SC administration to rats and monkeys. Non-adverse ALP201-related microscopic changes in monkey study were confined to the injection sites and included minimal to mild degeneration/necrosis in males and females. Degeneration/necrosis and mixed cell inflammation/infiltration of the injection site(s) were of lower incidence and severity or had features of chronicity (mineralization) within the recovery group, indicating partial recovery. Non-adverse microscopic changes in rat study were limited to the injection procedures with associated reactive changes in the draining lymph nodes. There was no discernible ALP201-associated alteration in the incidence, severity, or microscopic character of the changes at the injection sites or draining lymph nodes. Microscopic changes in the injection sites and draining lymph nodes of recovery animals were similar to those noted in the terminal necropsy animals, although less pronounced in the recovery group animals.

The observed exposure of the NOAEL at 20 mg/kg/dose from the GLP SC monkey toxicity study is approximately 113×(for IV) and 83×(for SC) higher than the projected AUC exposure of the proposed human starting single dose of 15 mg IV or 15 mg qw×3 SC (Table 18). Given the safety margin and the lack of systemic toxicity or local tolerability findings in the 28-day rat and monkey toxicology studies with ALP201, the safety risk for humans on the 15 mg starting dose is very low.

### Immunogenicity

Immunogenicity potential of ALP201 was evaluated by measuring ALP201 Anti-drug antibodies (ADA) in serum collected from the GLP rat and monkey 28-day toxicology studies.

In the rat toxicity study, the ADA responses were positive with SC administration in 9 of 20 (6 males; 3 females) animals from the control group, 10 of 19 (6 males; 4 females) animals at 2 mg/kg/dose, 12 of 20 animals (7 males; 5 females) at 10 mg/kg/dose and 12 of 20 animals (6 males; 6 females) at 30 mg/kg/dose on PK Day 28. Additionally, the ADA responses were positive with single dose IV ALP201administration in 14 of 20 animals (8 males; 6 females) at 10 mg/kg on PK Day 28. The positive ADA response did not appear to consistently impact the composite plasma concentration-time profiles of the 2 and 10 mg/kg/dose SC dose groups on Day 24 although the ADA may have contributed to the apparent decrease of systemic exposure at 30 mg/kg/dose on Day 24. The positive ADA responses in control animals are unlikely to be due to ALP201 mis-dosing based on the investigation conducted. A dose-dependent increase in ALP, an expected pharmacological effect of ALP201, was observed for the ≥2 mg/kg SC dose groups prior to terminal necropsy, which were resolved at recovery necropsy. The ALP results of animals in the control groups were similar prior to both terminal and recovery necropsy. Moreover, there were no ALP201-related in-life findings during the dosing and recovery phase. Therefore, taken collectively, the positive ADA responses observed in the control animals on PK Days 28 and 56 (study dates Day 29 and Day 57) were mostly likely due to a blood collection contamination and were not considered to have an impact on the study.

In the monkey toxicity study, the anti-ALP201 antibodies responses were negative for all pre-dose samples on Day 0. The ADA responses were positive in 4 of 10 animals at 1 mg/kg/dose, 6 of 10 animals at 5 mg/kg/dose, and 5 of 10 animals at 20 mg/kg/dose on Day 28. Although a direct correlation could not be made between positive ADA responses and systemic exposure concentration-time profiles as ADA samples were not sampled on TK collection days, following multiple doses, the concentration-time profiles for some of the animals at $5 mg/kg appeared to be impacted by anti-ALP201 antibodies. The animals impacted by anti-ALP201 antibodies were 3 animals (2 males and 1 female) at 1 mg/kg/dose, and 3 animals at 5 mg/kg/dose (3 females), with the days of impact on concentration-time profiles ranging from Day 18 to Day 24 for these animals.

### Example 2

The pharmacokinetic profile of asfotase alfa in multiple species suggests that a combination of reduced absolute bioavailability and half-life necessitates frequent dosing. The catalytic domain of human TNSALP is a highly glycosylated molecule. The presence of asialylated glycans leads to clearance via the asialoglycoprotein receptor (ASGPR) in the liver.

ALP201 was developed as a Next Generation alkaline phosphatase ERT with these considerations in mind. ALP201 retains the TNSALP-IgG Fc-D10 architecture used in asfotase alfa and incorporates the removal of two non-essential N-linked glycans, a change in the human Fc isotype to lgG2/4, and numerous process improvements in the expression of the molecule that lead to higher TSAC incorporation. In this report, we present the pharmacokinetic parameters for ALP201 determined in single dose studies of male C57BL/6 mice by intravenous and subcutaneous administration. We also report the PK parameters of multiple lots of ALP201 expressed and purified with varying levels of TSAC incorporation to help determine which PK parameters are most strongly affected by asialylated glycan clearance.

### Materials and Methods

### Animal Strain

To maintain consistency between these studies and previous pharmacokinetic studies performed on asfotase alfa, this study was performed using male C57BL/6 mice aged approximately 11-12 weeks at the time of dosing. The information for the test molecules is shown in Table 19.

TABLE 19
Test Articles

| | | | Total Sialic | | |
|---|---|---|---|---|---|
| | | Specific | Acid Content | Concentration | Formu- |
| Molecule | Lot # | Activity* | (mol/monomer) | (mg/mL) | lation |
| ALP201 | 1 | 36.9 | 5.9 | 8.35 | PBS |
| ALP201 | 2 | 32.8 | 5.9 | 9.15 | PBS |
| ALP201 | 3 | 40.3 | 5.0 | 3.09 | PBS |
| ALP201 | 4 | 39.1 | 3.2 | 6.54 | PBS |

*Specific activity listed in U/mg of 4-MUP hydrolysis, where 1 U = enzyme needed to hydrolyze 1 micromole of 4-MUP in 1 minute under assay buffer conditions at 37° C.

### Animal Dosing and Blood Sampling

Animals from 11-12 weeks of age were randomized by body weight and assigned to 4 groups. A 4 mg/kg dose of ALP201 was administered to 16 mice/group on day 0 by intravenous delivery (IV) (Group 1) or subcutaneously (SC) (Group 2). An equivalent weight-adjusted volume of PBS was administered on day 0 to 4 mice/group by IV (Group 3) or SC (Group 4). IV dosing was administered via tail vein. SC bolus dosing was administered in the scapular region above the shoulders.

A semi-serial sampling design was used, with 4 cohorts per group and n=4/per cohort+1 extra mouse per group. In the study run at Alexion, each mouse was bled 3 times, including the terminal bleed. Time-points (hours post-dose) are shown in Table 20 below for each cohort.

For non-terminal bleeds, 100 mL of whole blood was collected via submandibular collection into pre-coated lithium heparinized tubes to collect at least 50 μL of plasma per time point for each animal. At the terminal bleed, as much blood was collected as possible via cardiac puncture into lithium heparinized collection tubes. Blood was kept at 4° C. and processed for plasma by centrifugation as soon as feasible after collection. Each plasma sample was split into 2 equal volume aliquots prior to snap-freezing in CO₂/ethanol and storage at −80° C.

TABLE 20
Experimental Treatment Groups, Dose, Route of Administration
and Blood Sampling Schedule for In House Study

| | | | | | Dose | | |
|---|---|---|---|---|---|---|---|
| | No. of | Test | | Dose | Conc. | Dose | |
| Group | Mice | Article | Route | (mg/kg) | (mg/mL) | (mL/kg) | Vehicle |
| 1 | 16 + 1^{a} | ALP201 | IV | 4 | 1 | 4 | PBS- |
| | | | bolus | | | | without |
| | | | | | | | Ca/Mg |
| 2 | 16 + 1^{a} | ALP201 | SC | 4 | 1 | 4 | PBS- |
| | | | bolus | | | | without |
| | | | | | | | Ca/Mg |
| 3 | 4 | PBS | IV | — | — | — | — |
| | | | bolus | | | | |
| 4 | 4 | PBS | SC | — | — | — | — |
| | | | bolus | | | | |

^{a}N = 16 per group + 1 extra, which is used as a replacement animal in case of animal death or mis-dose

TABLE 21
Blood Sampling Schedule for In House Study, Cohorts of Groups 1 and 2

| | | | | 24 h | 48 h | 72 h | 96 h | 120 h | 192 h | 264 h | 336 h | 480 h |
|---|---|---|---|---|---|---|---|---|---|---|---|---|
| Cohort | 0.25 h | 1 h | 6 h | (D1) | (D2) | (D3) | (D4) | (D5) | (D8) | (D11) | (D14) | (D21) |
| 1 | X | | | | X | | | | X | | | |
| 2 | | X | | | | X | | | | X | | |
| 3 | | | X | | | | X | | | | X | |
| 4 | | | | X | | | | X | | | | X |

TABLE 22
Blood sampling Schedule for In House Study, Groups 3 and 4

| | | | | 24 h | 48 h | 72 h | 96 h | 120 h | 192 h | 264 h | 336 h | 480 h |
|---|---|---|---|---|---|---|---|---|---|---|---|---|
| Group | 0.25 h | 1 h | 6 h | (D1) | (D2) | (D3) | (D4) | (D5) | (D8) | (D11) | (D14) | (D21) |
| 3 | X | | | | | | | | X | | | |
| 4 | X | | | | | | | | X | | | |

For lots 2, 3, and 4, groups of 32 mice were assembled for each route of administration into 8 cohorts of 4 animals/timepoint. In groups with IV administration, each cohort was bled twice (the first time point as a survival collection, and the second terminally) at approximately 0.5, 1, 3, 8, 12, 21, 26, 32, 45, 49, 72, 96, 120, 192, 264, and 336 hours post-dose. In groups of SC administration, each cohort was bled twice (the first as a survival collection, and the second terminally) at approximately 1, 3, 6, 8, 12, 21, 26, 32, 45, 49, 72, 96, 120, 192, 264, and 336 hours post-dose.

### Determination of Active ALP201 Concentration in Plasma

Plasma samples were assayed for alkaline phosphatase activity using 4-methylumbelliferyl phosphate (4-MUP) as an artificial substrate. Hydrolysis of the phosphoester bond in 4-MUP releases the fluorescent compound 4-methylumbelliferone (4-MU), which is easily detected by a fluorimeter. Activity quantitation was performed via an enzyme activity standard curve.

### Sample Analysis for In House PK Study

Activity quantitation was performed via an enzyme activity standard curve created using serial dilutions of an asfotase alfa protein reference standard with known activity in the 4-MUP assay on the same plate. Thawed plasma samples were diluted 100-fold to 2,000-fold into assay buffer (50 mM HEPES, 150 mM NaCl, 1 mM MgCl₂, pH 7.4 and 1 mg/mL BSA) and assayed to determine active ALP201 enzyme concentration. Final protein standard range on each plate was from 4-80 ng/mL.

The diluted samples were quantitated as follows: all diluted samples were brought to 37° C. prior to initiation of the assay by addition of 4-MUP to the protein sample, resulting in a final concentration of 10 mM 4-MUP. Production of 4-MU was measured at an excitation wavelength of 360nm and an emission wavelength of 465 nm. Data was collected in a plate reader held at 37° C., with the plate read every 40 seconds for a total of 20 minutes. Rates of reaction for plasma and standard samples were calculated by linear regression of the reaction slope using Microsoft Excel in units of relative fluorescence units (RFU) per minute. Rates of reaction for standard samples were used to construct linear standard curve units in RFU/min/U. Comparison of measured rates of reaction in plasma samples were compared with the standard curve to determine plasma activity of ALP201 in U/mL. Sample activities in Units/mL were converted to mass units of mg/L by dividing the sample activities by the specific activity of the tested protein sample (in U/mg). Samples were run in duplicate twice to compile data for each independent sampling point.

### Sample Analysis for PK Studies

The same 4-MUP hydrolysis assay was performed. On the day of the assay, standards, quality controls (QCs), dilution controls (DCs), and blanks were diluted 250-fold in assay buffer. Standards, QCs, DCs, and blanks were further diluted 2-fold with the substrate, 4-Methylumbelliferyl phosphate (4-MUP), for the total 500-fold minimum required dilution (MRD). Prior to performing the MRD, the DC was diluted 200-fold in mouse plasma. ALP201 was quantified based on the fluorescent product methylumbelliferone, which results from the hydrolysis of 4-MUP substrate used in the assay. The plate was placed in a plate reader and read once every 60 seconds with kinetic fluorescence settings at 360 nm (excitation), 455 nm (cutoff), and 465 nm (emission) for 25 minutes at 37° C. The enzyme activity was directly proportional to the rate of the substrate reaction. The Vmax (rate in fluorescence intensity/min) was determined from a line of best fit through the data. The blank was not included in the curve fit. Results were reported in mg/L. Values were corrected for differences in specific activity between the test article and the protein activity standard.

### Pharmacokinetic Analysis

The PK analysis was performed using Phoenix WinNonlin v8.0 (Certara). Pharmacokinetic parameters were calculated using a noncompartmental analysis.

The following noncompartmental PK parameters were calculated: area under the concentration-time curve from time zero (dosing) to the last detectable concentration (AUCₜ), dose-normalized area under the concentration-time curve from time zero (dosing) to the last detectable concentration (AUCₜ/dose), area under the concentration-time curve from time zero (dosing) extrapolated to infinity (AUC_{inf}), dose-normalized area under the concentration-time curve from time zero (dosing) extrapolated to infinity (AUC_{inf}/dose), maximum observed plasma concentration (Cₘₐₓ), dose-normalized maximum observed plasma concentration (Cₘₐₓ/dose), time of maximum observed plasma concentration (Tₘₐₓ), terminal elimination half-life (t_{1/2}), total clearance (CL), apparent volume of distribution (V_{d}), and bioavailability (F). Actual sample collection times were used to calculate PK parameters. Actual doses were used to calculate dose-normalized PK parameters.

### Results and Discussion

### Pharmacokinetic Parameters of ALP201Following IV and SC Administration in Mice

The PK of ALP201 was evaluated following single IV or SC administration to male C57BL/6 mice. 16 mice were dosed in each group, and blood was collected from subdivided cohorts of 4 mice within each group at the following time points post-administration: 0.25, 1, 6, 24, 48, 72, 96, 120, 192, 264, 336, and 480 hours.

Plasma ALP201 concentrations were measured using an enzymatic activity assay, which measured alkaline phosphatase catalytic activity of ALP201. The enzymatic activity of ALP201 was then converted to mass unit (mg/L) for reporting.

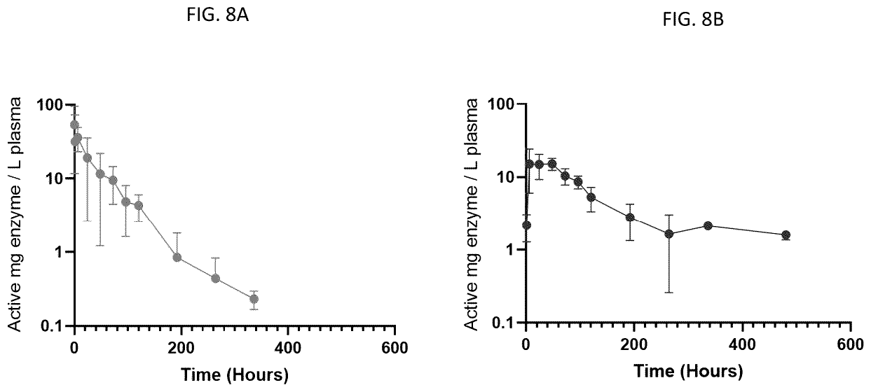

The mean active ALP201 plasma concentration versus time profiles following IV and SC administration are presented on semi-log scale in FIGS. 8A and 8B. PK parameters of ALP201 are summarized in Tables 7 and 11 above.

Individual mouse plasma activity profiles from some mice in the IV group appear to suggest that some of the dose may have been delivered subcutaneously, which may be responsible for the relatively high variability observed in early time points of the IV dosing profile. If this occurred, the calculated value of Cₘₐₓ for the IV dosed group may also be artificially low. After IV administration, the concentration-time profile declined in a multi-exponential manner (FIGS. 8A and 8B). The CL was 0.0019 L/h/kg and the apparent terminal t_{1/2} was 48 hours. After SC administration, the time to reach maximum concentration (tₘₐₓ) was 48 hours post-dose, suggesting relatively slow absorption from the SC injection site. Absolute bioavailability following SC administration was 96%.

### Pharmacokinetic Parameters of ALP201 lots with Varying Total Sialic Acid Content Values in Mice

Three separate lots of ALP201, purified with measured TSAC values of 5.9, 5.0, and 3.2 moles sialic acid/mol of protein monomer, were dosed by IV and SC administration into 32 mice. Blood was collected from subdivided cohorts of 4 mice within each administration group at the following time points post-administration: 0.5, 1, 3, 8, 12, 21, 26, 32, 45, 49, 72, 96, 120, 192, 264, and 336 hours post dose for IV administration; and 1, 3, 6, 8, 12, 21, 26, 32, 45, 49, 72, 96, 120, 192, 264, and 336 hours post dose for SC administration with each mouse bled twice.

Plasma ALP201 concentrations were measured using an assay, which measured alkaline phosphatase catalytic activity of ALP201. The enzymatic activity of ALP201 was reported in mass units (mg/L) by Charles River.

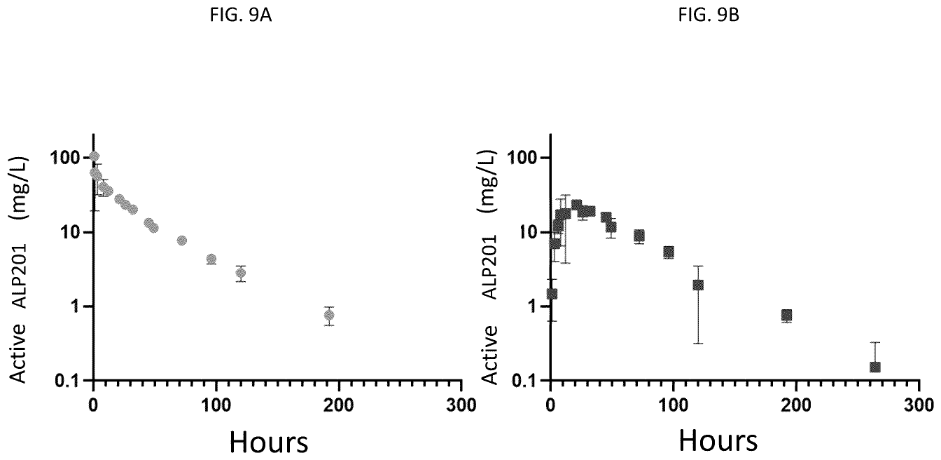

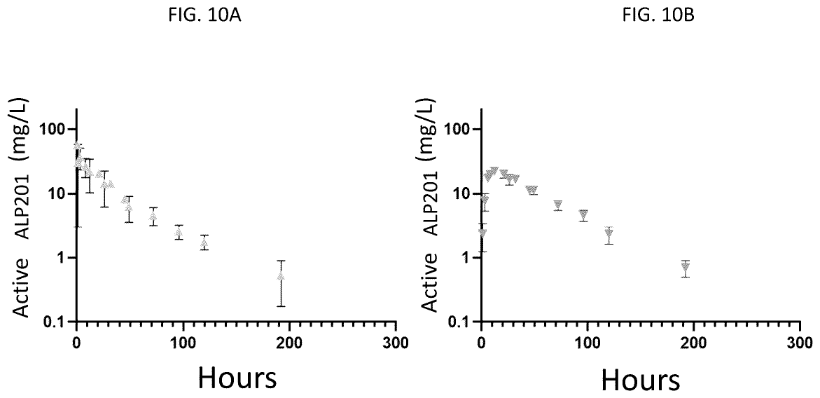

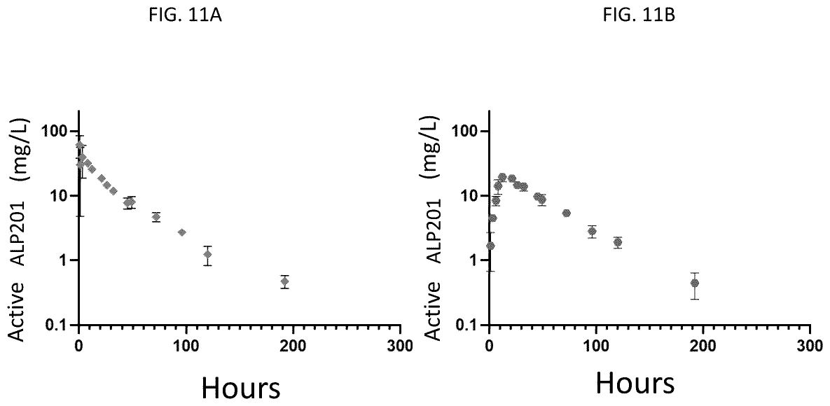

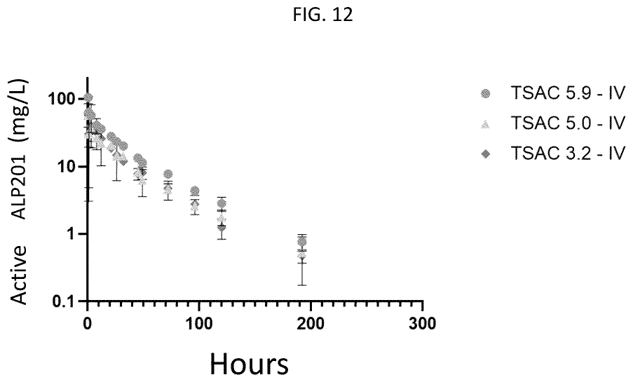

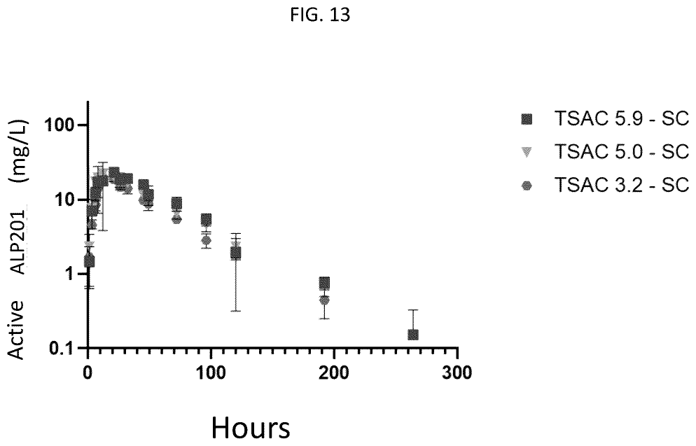

The mean active ALP201 plasma concentration versus time profiles following IV and SC administration for each protein lot are presented on semi-log scale in FIGS. 9A and 9B, 10A and 10B, and 11A and 11B. A collection of the plasma concentration versus time profiles for IV administration of the TSAC value variants is presented in FIG. 12. A collection of the plasma concentration versus time profiles for SC administration is presented in FIG. 13. Tabulated summaries of mean PK parameters and mean dose normalized PK parameters following IV and SC administration can be found in Tables 21 and 22 respectively.

TABLE 23
Summary of ALP201 Pharmacokinetic Parameters For TSAC Value Variants
Following Subcutaneous Administration in Male C57BL/6 Mice

| | | | | | | | | V_{d} or | CL or | |
|---|---|---|---|---|---|---|---|---|---|---|
| TSAC | | Dose | Cₘₐₓ | tₘₐₓ | t_{1/2} | AUCₜ | AUC_{∞} | V_{d}/F | CL/F | F |
| (mol/mol) | Route | (mg/kg) | (mg/L) | (h) | (h) | (mg*h/L) | (mg*h/L) | (L/kg) | (L/h/kg) | (%) |
| 5.9 | IV | 4 | 105.3 | 0.5 | 29.9 | 2060.5 | 2063.8 | 0.08 | 0.0019 | NA |
| 5.9 | SC | 4 | 23.3 | 21 | 33.4 | 1480.3 | 1487.6 | 0.13 | 0.0027 | 72.1 |
| 5.0 | IV | 4 | 57.3 | 0.5 | 31.4 | 1319.6 | 1322.4 | 0.14 | 0.0030 | NA |
| 5.0 | SC | 4 | 22.4 | 12 | 34.9 | 1316.5 | 1351.4 | 0.15 | 0.0030 | 102.2 |
| 3.2 | IV | 4 | 61.4 | 0.5 | 32.8 | 1308.1 | 1330.4 | 0.14 | 0.0030 | NA |
| 3.2 | SC | 4 | 19.7 | 12 | 35.6 | 1058.7 | 1081.5 | 0.19 | 0.0037 | 81.3 |

Abbreviations:
AUC_{∞} = area under the concentration time curve from time zero (dosing) extrapolated to infinity;
AUCₜ = area under the concentration time curve from time zero (dosing) to the last detectable concentration;
CL or CL/F = total clearance;
Cₘₐₓ = maximum observed plasma concentration;
F = absolute bioavailability;
IV = intravenous;
NA = not applicable;
SC = subcutaneous;
TSAC = total sialic acid content;
tₘₐₓ = time to maximum observed plasma concentration;
t_{1/2} = terminal elimination half-life;
V_{d} or V_{d}/F = apparent volume of distribution.

TABLE 24
Summary of Dose Normalized ALP201 Pharmacokinetic
Parameters For TSAC Value Variants Following Subcutaneous
Administration in Male C57BL/6 Mice

| | | Cₘₐₓ/dose | AUCₜ/dose | AUC_{∞}/dose |
|---|---|---|---|---|
| TSAC | | [(mg/L)/ | [(mg* h/L)/ | [(mg * h/L)/ |
| (mol/mol) | Route | (mg/kg)] | (mg/kg)] | (mg/kg)] |
| 5.9 | IV | 26.3 | 515.1 | 516.0 |
| 5.9 | SC | 5.8 | 371.1 | 371.9 |
| 5.0 | IV | 14.3 | 329.9 | 330.6 |
| 5.0 | SC | 5.6 | 329.1 | 337.9 |
| 3.2 | IV | 15.4 | 327.0 | 332.6 |
| 3.2 | SC | 4.9 | 264.7 | 270.4 |

Abbreviations:
AUC_{inf}/dose = dose normalized area under the concentration time curve from time zero (dosing) extrapolated to infinity;
AUCₜ/dose = dose normalized area under the concentration time curve from time zero (dosing) to the last detectable concentration;
Cₘₐₓ/dose = dose normalized maximum observed plasma concentration;
IV = intravenous;
SC = subcutaneous;
TSAC = total sialic acid content

ALP201 protein lots with TSAC values of 5.9, 5.0, and 3.2 mol/mol, correspond to 1.48, 1.25, and 0.80 moles of sialic acid per consensus N-linked glycan site, and the PK profiles of all lots (for both IV and SC administration) were similar in shape, with similar Tₘₐₓ and t_{1/2} values.

With increasing TSAC value, clearance slightly decreased, Cₘₐₓ and AUC by both IV and SC routes increased, and volume of distribution decreased. All of these changes are consistent with the idea that lower TSAC values lead to higher levels of immature N-linked glycans, which can be quickly cleared by cellular receptors like ASGPR and cause higher clearance, lower Cₘₐₓ and lower AUC for a protein sample. Changes in these parameters are relatively small, and maximally differ by 2-fold at most.

### Comparison of ALP201 PK Parameters in Mice with Asfotase Alfa PK Parameters in Mice

As a second-generation alkaline phosphatase ERT, ALP201 was designed to improve upon the PK parameters of asfotase alfa, which was previously tested in mouse PK studies using male C57BL/6 mice. A summary of the PK parameters for asfotase alfa is presented in Table 25.

TABLE 25
Previously Determined Pharmacokinetic Parameters of Asfotase Alfa
in C57BL/6 mice after a Single IV or SC Administration at 2 mg/kg

| | Cₘₐₓ | Cmax/dose | t_{1/2} | AUC_{t (0-49)} | AUC_{∞} | AUC_{∞}/dose | CL | F |
|---|---|---|---|---|---|---|---|---|
| Route | (mg/L) | [(mg/L)/(mg/kg)] | (h) | (mg*h/L) | (mg*h/L) | [(mg*h/L)/(mg/kg)] | (L/h/kg) | (%) |
| IV | 26.4 | 13.2 | 15.6 | 258 | 286 | 143 | 0.007 | NA |
| SC | 3.07 | 1.54 | 31.1 | 100 | 161 | 81 | ND | 38.8 |

Abbreviations:
AUC_{∞} = area under the concentration time curve from time zero (dosing) extrapolated to infinity;
AUCₜ = area under the concentration time curve from time zero (dosing) to the last detectable concentration;
CL = total clearance;
Cₘₐₓ = maximum observed plasma concentration;
F = absolute bioavailability;
IV = intravenous;
SC = subcutaneous;
t_{1/2} = terminal elimination half-life

In all PK data sets collected for ALP201 in male C57BL/6 mice, ALP201 demonstrates superior in vivo half-life (as measured by t_{1/2}), in vivo exposure (as measured by Cₘₐₓ and AUC), and bioavailability. For all tested samples, ALP201 also demonstrates significantly lower clearance in mice than asfotase alfa

Because asfotase alfa release specifications limit the molecule to TSAC values of 1.2-3.0 mol/mol and the molecule has 6 N-linked glycosylation sites per monomer, sialic acid incorporation for asfotase alfa is only 0.20-0.50 moles sialic acid/glycan. This indicates that many glycans on asfotase alfa contain no sialic acid moieties and could be efficient substrates for ASPGR clearance. This could explain the higher clearance, and lower dose normalized Cmax and AUC observed for asfotase alfa relative to ALP201.

These samples of ALP201 all have much higher bioavailability than asfotase alfa, with the least bioavailable sample of ALP201 having roughly double the observed bioavailability of asfotase alfa (72.1% vs. 38.8%). Differences in the human IgG Fc domain isotype between the two constructs may be the cause of this observation. We have shown that the IgG2/4 Fc domain of ALP201 does not bind to a panel of common Fcg receptors, but the IgG1 Fc domain of asfotase alfa does bind strongly to at least two of them (Source: Research Technical Report 036). If these Fcg receptors are present in the subcutaneous space, asfotase alfa may be cleared before it even enters systemic circulation, lowering bioavailability and exposure.

### Conclusion

The findings from these studies indicate that ALP201 has a significantly improved dose-adjusted pharmacokinetic profile in mice relative to asfotase alfa following either IV or SC administration, with higher bioavailability, dose-normalized Cₘₐₓ and dose-normalized exposure. Additional PK studies using ALP201 samples with varying levels of TSAC incorporation indicated small increases in Cₘₐₓ and in vivo exposure with increased TSAC levels. Elimination half-life, Tₘₐₓ, and bioavailability appeared to be relatively unaffected by changes in TSAC values.

### Example 3

### Overview of Single Dose PK Studies with ALP201

Two single dose PK studies were conducted as part of the asfotase alfa non-clinical development program, in Sprague-Dawley rat and Cynomolgus monkey, respectively (Table 26). These studies supported ALP201 single dose studies.

TABLE 26
Single Dose PK Studies Conducted with Asfotase Alfa

| | | | Route of | |
|---|---|---|---|---|
| Name of | | | Adminis- | |
| Study | Duration | Species | tration | Formulation |
| Single | 3 days | Rat | IV, SC | 25 mM sodium |
| dose PK | | | | phosphate pH 7.4, |
| | | | | 150 mM sodium chloride |
| Single | 3 days | Monkey | IV, SC | 25 mM sodium |
| dose PK | | | | phosphate pH 7.4, |
| | | | | 150 mM sodium chloride |

Note:
Unless otherwise stated, PK/TK parameters were characterized by the non-compartmental analysis (NCA) method.
Abbreviations:
IV = intravenous;
PK = pharmacokinetics;
SC = subcutaneous;
TK = toxicokinetics.

Following single dose IV administration, systemic clearance (CL) of asfotase alfa ranged from 0.0193-0.0492 L/h/kg with apparent half-life ranging from 27-34 hours. Volume of distribution at steady state (Vₛₛ) ranged from 0.432-1.39 L/kg. Following single SC administration to rats and monkeys, the time to reach maximum concentration (Tₘₐₓ) ranged from 10-32 hours post-dosing, suggesting slow absorption of asfotase alfa from the SC injection site. The estimated bioavailability for the SC route of administration ranged from 26%-35% in rats and monkeys.

### Conclusions from Single Dose PK Studies

ALP201 has been designed to improve upon the characteristics of the approved product, asfotase alfa, with improved efficacy/activity, systemic exposure, absolute bioavailability, and half-life. The Ig Fc domain isotype in ALP201 is G2/4 instead of the IgG1 Fc domain present in asfotase alfa. This alteration is designed to improve ALP201's pharmacokinetic properties including systemic PK exposure, half-life, and bioavailability.

Preclinical data from 3 animal species (mouse, rat, and monkey) provide supportive evidence that these objectives have been achieved. In comparison to asfotase alfa, ALP201 has demonstrated significant improvement in PK exposure (>10-fold), absolute bioavailability (2-fold), and half-life (2-fold) following IV/SC administration in 3 animal species. Preclinical population PK modeling also predicts that improved ALP201 PK properties are expected to extrapolate to human, which will facilitate treatment of patients with reduced injection volume, less frequent administration, and lower total amount on an annual basis, thereby reducing the burden of treatment for patients.

### Nonclinical Safety Studies

### Summary of Toxicology Studies to Support First-in-Human Study

Standalone single dose toxicology/tolerability studies were not conducted for ALP201. ALP201 doses for repeated dose definitive (GLP) toxicology studies were selected based on the data obtained from ALP201 single dose IV and SC PK studies in rats and monkeys. The tolerability of ALP201 was assessed in the single dose IV and SC PK studies in rats and monkeys by clinical observations, including site of injection reactions and clinical pathology data. In summary, ALP201 was well tolerated when a single dose was administered via either IV or SC in rats up to 27 mg/kg and in monkeys up to 20 mg/kg.

Definitive GLP-compliant ALP201 28-day toxicity studies in cynomolgus monkeys and Sprague-Dawley rats are ongoing. The in-life portion of rat and monkey studies, including the recovery phases, are complete and the reports are being prepared. Although audited draft reports of these studies are not yet available, all data from both studies except ADA data are available and the findings are summarized in Table 27. In summary, the results from ALP201 28-day toxicity studies in rats and monkeys indicate that ALP201 treatment did not result in any noteworthy/biologically meaningful treatment-related adverse effects from any of the toxicity endpoints evaluated in both the studies, including safety pharmacology endpoints.

TABLE 27
Comparison of Findings from ALP201 Rat and Monkey 28-Day GLP Toxicity Studies

| | ALP201 Monkey GLP Toxicity Study^{a} | ALP201 Rat GLP Toxicity Study^{a} |
|---|---|---|
| Age of Animals | Approximately 2-3 years | Approximately 7-8 weeks |
| Duration of Study | 28-Days | 28-Days |
| Recovery Period | 28-Days | 28-Days |
| Route of | Subcutaneous injection | Subcutaneous injection^{b} |
| Administration | | |
| Frequency of | Once every 3 days (Q3D) | Once every 3 days (Q3D) |
| Administration | | |
| Total number of | 10 doses | 10 doses |
| doses administered | | |
| Doses administered | 0, 1, 5, or 20 mg/kg/dose | 0, 2, 10, or 30 mg/kg/dose |
| NOAEL | 20 mg/kg/dose | 30 mg/kg/dose |
| AUC₀₋ₜ^{c} at NOAEL | Day 1: 27,900 μg · hr/mL | Day 1: 11,600 μg · hr/mL |
| | Day 24: 36,800 μg · hr/mL | Day 24: 4,380 μg · hr/mL |
| Cₘₐₓ at NOAEL^{c} | Day 1: 125 μg/mL | Day 1: 79.9 μg/mL |
| | Day 24: 254 μg/mL | Day 24: 43.9 μg/mL |
| Mortality | No mortality | No mortality |
| Clinical Observations | No treatment-related clinical | No treatment-related clinical |
| | observations | observations |
| Body Weight | No treatment-related findings | No treatment-related findings |
| Food Consumption | No treatment-related findings | No treatment-related findings |
| Ophthalmology | No treatment-related findings | No treatment-related findings |
| Dermal Observations | No treatment-related findings at | No treatment-related findings at site of |
| | site of injections | injections |
| ECG | No treatment-related findings | NA |
| | based on quantitative parameters | |
| | (heart rate, RR interval, PR | |
| | interval, QRS duration, QT interval, | |
| | and QTc interval) and qualitative | |
| | review of the ECG | |
| Blood Pressure | No treatment-related findings | NA |
| Respiratory | No treatment-related findings | No treatment-related findings |
| CNS | NA | No treatment-related findings |

Clinical Pathology Parameters

| | | |
|---|---|---|
| Hematology | No treatment-related findings | No treatment-related findings |
| Coagulation | No treatment-related findings | No treatment-related findings |
| Clinical Chemistry | No treatment-related findings apart | No treatment-related findings apart from |
| | from dose dependent increases in | dose dependent increases in ALP levels, |
| | ALP levels, an expected | an expected pharmacological effect |
| | pharmacological effect | |
| Urinalysis | No treatment-related findings | No treatment-related findings |
| Organ Weights | No treatment-related findings | No treatment-related findings |
| Macroscopic | No treatment-related findings | No treatment-related findings |
| Findings | | |
| Anatomic Pathology | Slight degeneration/necrosis and | Sporadic injection site findings with |
| | mineralization of injection sites | associated reactive changes in the |
| | were observed; but were | draining lymph nodes; these findings |
| | concluded to be non-adverse. | were not dose dependent and were |
| | These findings were partially | concluded to be non-adverse; these |
| | recovered at the end of recovery. | findings were partially reversed at the |
| | No other micro or macroscopic | end of recovery. No other micro or |
| | observations were noted in the | macroscopic observations were noted in |
| | study. | the study. |
| Immunogenicity | Data not available yet | Data not available yet |

^{a}Rat and monkey 28-day toxicity studies are in reporting phase.
^{b}In rat study single-dose of ALP201 was administered at 10 mg/kg intravenously to Group 5 animals, to determine the absolute bioavailability of ALP201 following SC administration.
^{c}Gender-combined mean systemic exposure values are reported. AUC values for the IV and SC studies are reported as AUC_{0-24.5} and AUC₀₋₁₆₈, respectively.
Abbreviations:
ALP = alkaline phosphatase;
AUC = area under the concentration-time curve;
Cₘₐₓ = maximum observed plasma concentration;
CNS = central nervous system;
ECG = electrocardiogram;
GLP = good lab practices;
IV = intravenous;
NOAEL = no-observed adverse effect level;
Q3D = every 3 days
SC = subcutaneous.

### Nonclinical Safety Strategy for ALP201 Late-Stage Clinical Development

ALP201 is an improved ERT relative to Alexion's marketed ERT product asfotase alfa (STRENSIQ®). The human TNSALP catalytic domain of ALP201 includes rationally designed changes at 3 positions to confer a higher enzymatic activity. The Fc part of ALP201 is a human immunoglobulin gamma 2/4 (IgG2/4), while the Fc portion of asfotase alfa is a human immunoglobulin IgG1. The C-terminal regions of ALP201 and asfotase alfa, which target the bone, are identical.

The available findings from ALP201 and selected asfotase alfa studies are as follows:
1. The toxicity findings from ALP201 GLP 28 days toxicology studies in rats and monkeys are very similar (Table 31)
2. The toxicity findings from ALP201 and asfotase alfa toxicology studies of similar duration (4 weeks/28 days) in monkeys and rats are very similar (Table 32 and Table 33, respectively)
3. The toxicity findings from the asfotase alfa complete nonclinical safety package are largely limited to transient local tolerability findings in rats, with no other noteworthy adverse asfotase alfa-treatment related findings.

### ALP201 Chronic/6 Months General Toxicology Studies Strategy

A single species chronic toxicology study strategy is supported by the following observations:
4. No noteworthy systemic toxicity and/or local tolerability findings were observed in rat and monkey 28-day GLP toxicology studies, following 10 repeated administrations of SC ALP201 once every 3 days (Q3D), although the systemic exposures of ALP201 were significantly higher compared to asfotase alfa. The systemic exposures are summarized in Tables 31 and 32
5. The toxicity findings from ALP201 rat and monkey 28-day GLP toxicology studies were very similar. The findings from these studies are summarized in Table 28
6. If the toxicological findings from short term general toxicology studies are similar, then longer-term general toxicity studies in 1 species are usually considered sufficient.
7. Rat is the preferred species for chronic toxicity study with ALP201. Additionally, rat is the preferred species when only one species is used to conduct longer term general toxicity studies.

TABLE 28
Comparison of Findings From Asfotase Alfa and ALP201 Monkey GLP Toxicity Studies

| | Asfotase Alfa Monkey GLP | Asfotase Alfa Monkey GLP | ALP201 Monkey GLP |
|---|---|---|---|
| | Toxicity Study | Toxicity Study | Toxicity Study |
| Age of Animals | Approximately 1 year (Juvenile | Approximately 1 year (Juvenile | Approximately 2-3 years |
| | monkeys) | monkeys) | |
| Duration of Study | 4 Weeks | 6 Months (26 Weeks) | 4 Weeks |
| Recovery Period | 28 Days | 28 Days | 28 Days |
| Route of Administration | Intravenous injection | Subcutaneous injection | Subcutaneous injection |
| Frequency of | Once weekly | Once daily | Once every 3 days (Q3D) |
| Administration | | | |
| Total Number of Doses | 4 doses (Days 1, 8, 15, and 22) | 182 doses | 10 doses |
| Administered | | | |
| Doses administered | 0, 5, 15, or 45 mg/kg/dose | 0, 0.43, 2.14, or 10 mg/kg/day | 0, 1, 5, or 20 mg/kg/dose |
| NOAEL | 45 mg/kg/dose | 10 mg/kg/day | 20 mg/kg/dose |
| AUC₀₋₁₆₈ₕ at NOAEL | Day 1: 3,190 μg · hr/mL | Day 1: 50 μg · hr/mL^{a} | Day 1: 27,900 μg · hr/mL |
| | Day 22: 2,670 μg · hr/mL | Week 26: 124 μg · hr/mL^{a} | Day 24: 36,800 μg · hr/mL |
| Cₘₐₓ at NOAEL | Day 1: 726 μg/mL | Day 1: 2.66 μg/mL | Day 1: 125 μg/mL |
| | Day 22: 691 μg/mL | Week 26: 6.68 μg/mL | Day 24: 254 μg/mL |
| Mortality | No mortality | No mortality | No mortality |
| Clinical Observations | No treatment-related clinical | No treatment-related clinical | No treatment-related clinical |
| | observations | observations | observations |
| Body Weight | No treatment-related findings | No treatment-related findings | No treatment-related findings |
| Food Consumption | No treatment-related findings | No treatment-related findings | No treatment-related findings |
| Ophthalmology | No treatment-related findings | No treatment-related findings | No treatment-related findings |
| Dermal Observations | No treatment-related findings | Injection site reaction at all doses | No treatment-related findings at site |
| | | include skin scabs, dry and/or red | of injections |
| | | skin | |
| ECG | No treatment-related findings based | No treatment-related findings based | No treatment-related findings based |
| | on quantitative parameters (heart | on quantitative parameters (heart | on quantitative parameters (heart |
| | rate, RR interval, PR interval, QRS | rate, RR interval, PR interval, QRS | rate, RR interval, PR interval, QRS |
| | duration, QT interval, and QTc | duration, QT interval, and QTc | duration, QT interval, and QTc |
| | interval) and qualitative review of the | interval) and qualitative review of the | interval) and qualitative review of the |
| | ECG | ECG | ECG |
| Blood Pressure | No treatment-related findings | No treatment-related findings | No treatment-related findings |

Clinical Pathology

| | | | |
|---|---|---|---|
| Hematology | No treatment-related findings | No treatment-related findings | No treatment-related findings |
| Coagulation | No treatment-related findings | No treatment-related findings | No treatment-related findings |
| Clinical Chemistry | No treatment-related findings apart | No treatment-related findings apart | No treatment-related findings apart |
| | from dose dependent increases in | from dose dependent increases in | from dose dependent increases in |
| | ALP levels, a pharmacological effect | ALP levels, a pharmacological effect | ALP levels, a pharmacological effect |
| Urinalysis | No treatment-related findings | No treatment-related findings | No treatment-related findings |
| Organ Weights | No treatment-related findings | No treatment-related findings | No treatment-related findings |
| Macroscopic Findings | No treatment-related findings | No treatment-related findings | No treatment-related findings |
| Anatomic Pathology | No treatment-related findings | Injection sites had focal | Slight degeneration/necrosis and |
| | | granulomatous inflammation and | mineralization of injection sites were |
| | | mononuclear infiltration at doses ≥0.43 | observed; but were concluded non- |
| | | mg/kg/dose that was partially to | adverse. These findings were |
| | | completely recovered at the end of | partially recovered at the end of |
| | | recovery period; | recovery. No other micro or |
| | | Slight degeneration or necrosis of | macroscopic observations were |
| | | muscle underneath the site of | noted in the study. |
| | | injection | |
| | | in one male monkey at 2.14 | |
| | | mg/kg/dose | |

^{a}AUC values for Study 670338 are reported as AUC₀₋₂₄ₕ
Abbreviations:
ALP = Alkaline phosphatase;
AUC = area under the concentration-time curve;
ADA = anti-drug antibody;
Cₘₐₓ = maximum observed plasma concentration;
ECG = electrocardiogram;
GLP = good lab practices;
NOAEL = no-observed adverse effect level;
Q3D = every 3 days.

TABLE 29
Comparison of Findings From Asfotase Alfa and ALP201 Rat GLP Toxicity Studies

| | Asfotase Alfa Rat GLP | Asfotase Alfa Rat GLP | ALP201 Rat GLP |
|---|---|---|---|
| | Toxicity Study | Toxicity Study | Toxicity Study |
| Age of Animals | Approximately 21-24 days (Juvenile | Approximately 21 days (Juvenile | Approximately 7-8 weeks |
| | rats) | rats) | |
| Duration of Study | 4-Weeks | 6-Months | 4-Weeks |
| Recovery Period | 28-Days | 28-Days | 28-Days |
| Route of | Intravenous injection | Intravenous injection | Subcutaneous injection |
| Administration | | | |
| Frequency of | Once weekly | Once daily | Once every 3 days (q3d) |
| Administration | | | |
| Total Number of Doses | 4 doses (Days 1, 8, 15, and 22) | 182 doses | 10 doses |
| Administered | | | |
| Doses administered | 0, 3, 30, or 90 mg/kg/dose (Nominal | 0, 1, 3, or 13 mg/kg/day | 0, 2, 10, or 30 mg/kg/dose |
| | doses) | | |
| | 0, 2.6, 26, or 77 mg/kg/dose (Actual | | |
| | measured doses) | | |
| NOAEL | 26 mg/kg/dose | 13 mg/kg/day | 30 mg/kg/dose |
| AUC₀₋₁₆₈^{a} at NOAEL | Day 23: NA | Week 1: 364 μg · hr/mL | Day 1: 11,600 μg · hr/mL |
| | | Week 26: 379 μg · hr/mL | Day 24: 4,380 μg · hr/mL |
| Cₘₐₓ at NOAEL | Day 23: 3.79 μg/mL | Week 1: 115 μg/mL | Day 1: 79.9 μg/mL |
| | | Week 26: 281 μg/mL | Day 24: 43.9 μg/mL |
| Mortality | 1 rat (concluded non treatment- | 9 rats (all 9 deaths were concluded | No mortality |
| | related) | non treatment-related) | |
| Clinical Observations | Acute/transient injection reactions (up | Acute dose-dependent swelling of | No treatment-related clinical |
| | to 60 minutes post-dose) at all doses; | extremities (fore and hind paws and | observations |
| | no injection reactions were observed | muzzle); these swelling subsided | |
| | during recovery period in all groups or | in <24 hours post-dose | |
| | vehicle control group during treatment | | |
| | period | | |
| Body Weight | Dose-dependent slight decreases | No treatment-related findings | No treatment-related findings |
| | (7.4-12.1%) in mean body weight gain | | |
| | in males during recovery period | | |
| Food Consumption | Dose-dependent slight decreases in | No treatment-related findings | No treatment-related findings |
| | food consumption in males correlating | | |
| | with decreases in bodyweight gains | | |
| Ophthalmology | No treatment-related findings | No treatment-related findings | No treatment-related findings |
| Dermal Observations | No treatment-related findings | Injection site reaction at all doses | No treatment-related findings at site |
| | | include skin scabs, dry and/or red | of injections |
| | | skin | |
| CNS | No treatment-related findings | No treatment-related findings | No treatment-related findings |
| Estrous Cycle | No treatment-related findings | No treatment-related findings | No treatment-related findings |

Clinical Pathology

| | | | |
|---|---|---|---|
| Hematology | At high dose slight decreases in | No treatment-related findings | No treatment-related findings |
| | neutrophils, monocytes and | | |
| | eosinophils; slight increases in | | |
| | lymphocytes, platelets, and | | |
| | reticulocytes relative to control group | | |
| Coagulation | No treatment-related findings | No treatment-related findings | No treatment-related findings |
| Clinical Chemistry | No treatment-related findings apart | No treatment-related findings apart | No treatment-related findings apart |
| | from dose dependent increases in | from dose dependent increases in | from dose dependent increases in |
| | ALP levels, a desired pharmacological | ALP levels, a desired | ALP levels, a desired |
| | effect | pharmacological effect | pharmacological effect |
| Urinalysis | No treatment-related findings | No treatment-related findings | No treatment-related findings |
| Organ Weights | No treatment-related findings | No treatment-related findings | No treatment-related findings |
| Macroscopic Findings | No treatment-related findings | No treatment-related findings | No treatment-related findings |
| Anatomic Pathology | No treatment-related findings | No treatment-related findings | Sporadic injection site findings with |
| | | | associated reactive changes in the |
| | | | draining lymph nodes; these findings |
| | | | were not dose dependent and were |
| | | | concluded to be non-adverse; these |
| | | | findings were partially reversed at the |
| | | | end of recovery. No other micro or |
| | | | macroscopic observations were |
| | | | noted in the study. |
| Immunogenicity | The screening ADA assay | No noteworthy ADA response | Data not available yet |
| | demonstrated ADA in all dose groups | observed | |
| | including vehicle (Day 1 67% and | | |
| | Day 28 100%) and treatment groups | | |
| | (Day 1 56 to 89% and Day 28 | | |
| | 100%); no confirmatory ADA assay | | |
| | was conducted due to low sample | | |
| | volume availability. | | |

^{a}Gender-combined mean systemic exposure values are reported. AUC values are reported as AUC₀₋₁₆₈
Abbreviations:
ALP = Alkaline phosphatase;
AUC = area under the concentration-time curve;
ADA = anti-drug antibody;
Cₘₐₓ = maximum observed plasma concentration;
CNS = central nervous system;
GLP = good lab practices;
IV = intravenous;
MTD = maximum tolerated dose;
NOAEL = no-observed adverse effect level;
Q3D = every 3 days;
SC = subcutaneous.

### Model-Based Analyses and Predicted Human Dose Regimens

### ALP201 Modeling

### Data used in Current Models

To perform dose extrapolation from mouse (disease model species) to human, sufficient PK analysis is required to simulate human exposures from allometrically-scaled doses. Limited mouse PK data was available; a single dose wildtype mouse study (HPP-PK-01) and single C_{trough} measurements (D36/D37) from 2 multiple dose Akp2GW −/− mouse efficacy studies (HPP-PoC-01 and HPP-MED-01). To create a robust PK characterization of ALP201, the mouse data was enriched with PK data from dose range-finding studies in rats and cynomolgus monkeys. Table 30 shows the data (47 animals and 387 PK concentrations), to date, analyzed using a population pharmacokinetic (Pop-PK) modeling approach. For the dose-response modeling, efficacy (bone mineralization) data from 2 Akp2GW −/− mouse efficacy studies (HPP-PoC-01 and HPP-MED-01) were used.

TABLE 30
ALP201 Single Dose PK Data Used to Develop the Pop-PK Model

| | | ALP201 Single Dose, mg/kg; N (measurements) | | | | | | | | |
|---|---|---|---|---|---|---|---|---|---|---|
| Species | Route | 1 | 2 | 3 | 4 | 6 | 9 | 20 | 27 | Total |
| Mouse | IV | • | • | • | 4 (30) | • | • | • | • | • |
| | SC | • | • | • | 4 (37) | • | • | • | • | • |
| Rat | IV | 4 (33) | • | 4 (33) | • | • | 4 (36) | • | 4 (35) | • |
| | SC | • | 4 (24) | • | • | • | • | • | 4 (35) | • |
| Monkey | IV | • | 3 (27) | • | • | 3 (26) | • | 3 (24) | • | • |
| | SC | • | 3 (24) | • | • | • | • | 3 (24) | • | • |
| Total | | • | • | • | • | • | • | • | • | 47 (387) |

Abbreviations:
IV = intravenous;
N = number of animals;
PK = pharmacokinetic(s);
Pop-PK = population-pharmacokinetics;
SC = subcutaneous.

### Current Pop-PK Model

The current Pop-PK model was developed using the NONMEM software program, version 7.2 (ICON solutions) to simultaneously analyze PK data from 3 animal species accounting for species differences in PK disposition using bodyweight based allometric principles, where animal body weight was centered to 70 kg and allometric exponents were fixed to 0.75 for clearance parameters and 1.0 for volume of distribution parameters. The IV and SC data were also fit simultaneously to estimate extravascular parameters, e.g., absolute bioavailability, following SC injection. The current model is a two-compartment model with linear elimination and model parameter estimates are in Table 31.

TABLE 31
Parameter Estimates for the Interim Pop-PK Model

| | Typical | RSE | BSV | RSE | Shrinkage |
|---|---|---|---|---|---|
| Parameter, Units | Value | (%) | (%) | (%) | (%) |
| Clearance, CL, L/h/70 kg | 0.025 | 5 | 27 | 24 | 10 |
| Central Volume, V2, L/70 kg | 3.48 | 5 | . | . | . |
| Periph Volume, V3, L/70 kg | 2.71 | 13 | . | . | . |
| Periph CL, Q, L/h/70 kg | 0.027 | 36 | . | . | . |
| Absorption Rate, Ka, 1/h | 0.031 | 25 | 86 | 41 | 40 |
| Bioavailability, F, % | 74.6 | 11 | 76 | 76 | 59 |
| Proportional residual | 24.6 | 38 | . | . | 7 |
| error; RAT, % | | | | | |
| Additive residual | 0.707 | 29 | . | . | 7 |
| error; RAT, ug/mL | | | | | |
| Proportional residual | 30.4 | 36 | . | . | 7 |
| error; CYNO, % | | | | | |
| Proportional residual | 47 | 31 | . | . | 5 |
| error; MOUSE, % | | | | | |

Abbreviations:
BSV = between subject variability;
RSE = relative standard error (parameter precision from $COV step)

The typical value or mean parameter estimates for the PK parameters were sufficiently estimated. The variability (BSV) for CL was acceptable and well estimated; however, the variability for Ka and F was large (86% and 76%, respectively) and not well estimated. Shrinkage is an assessment of how the data informed the estimation of PK parameters with a value of <30% being good. Given the high shrinkage values for Ka and F (40% and 59%, respectively), additional data from the 4-week GLP toxicology studies should improve the estimation of the Ka and F parameters. Overall, the current Pop-PK model should provide reasonable dose simulations given the good mean parameter estimates; although, the variability region may be overly wide.

### Current Dose-Response Model

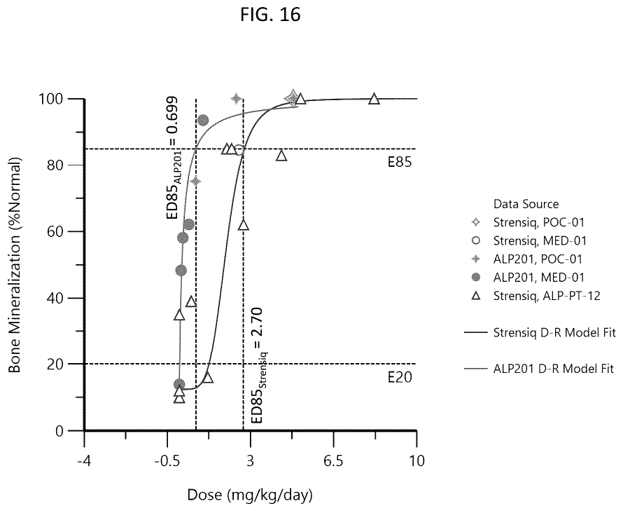

The developed dose-response model was selected by testing models from the Eₘₐₓ family. Most dose-response relationships can be described by one of the Eₘₐₓ model parameterizations. The current best dose-response characterization is using an Eₘₐₓ+E₀ (baseline) model. The efficacy endpoint, bone mineralization, was a radiographic assessment, and was selected as it shared the same clinical definition of efficacy as that used for asfotase alfa nonclinical dose-response assessment. Bone mineralization after treatment with asfotase alfa or ALP201 from 2 efficacy studies (HPP-PoC-01 and HPP-MED-01) was plotted versus dose (FIG. 16). Asfotase alfa data from previous efficacy study ALP-PT-12 was included for comparison. The y-axis was expressed as % Normal, which was defined as the percentage of mice in a dose group with bone mineralization scores of 4. The x-axis was expressed as dose normalized to mg/kg/day. The dose producing normal mineralization in 85% of the treated population (ED85) was chosen as the target effective dose.

### Dose Translation from Mouse to Human and Proposed Human Starting Doses

Allometric scaling was applied to mouse target effective dose (ED85) to determine a human dose. The equation to predict from mouse ED85 to human equivalent dose (HED, mg/kg/day) was ED85×(0.025 kg mouse/70 kg human)^{0.25}. The HED was translated into a weekly flat dose of 45 mg/week and used as the starting dose for the MAD arm of the FIH study. The maximum recommended starting dose (MRSD) for the SAD arm was selected after considering results from the NOAEL and MABEL methods. It was decided that the studied dose that produced the minimum on-target pharmacological response would be selected and translated to a flat human dose (5 mg).

### Other Embodiments

The foregoing detailed description and examples have been given for clarity of understanding only. No unnecessary limitations are to be understood therefrom. The disclosure is not limited to the exact details shown and described, for variations apparent to one skilled in the art will be included within the scope defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

The complete disclosures of all patents, patent applications including provisional patent applications, publications including patent publications and nonpatent publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The disclosure is not limited to the exact details shown and described, for variations apparent to one skilled in the art will be included within the embodiments defined by the claims.

1. A method of treating a bone mineralization disorder in a subject in need thereof comprising administering a pharmaceutical composition comprising a dimer comprising a first polypeptide and a second polypeptide, each polypeptide comprising the amino acid sequence of SEQ ID NO: 5, wherein the dimer is linked by a first disulfide bond between C494 of the first polypeptide and C494 of the second polypeptide and a second disulfide bond between C497 of the first polypeptide and C497 of the second polypeptide, wherein the pharmaceutical composition comprises a pharmaceutically acceptable carrier comprising phosphate and sucrose.

2. The method of claim 1, wherein the pharmaceutical composition comprises:
(a) from about 1 mM to about 100 mM phosphate; and/or
(b) from about 1mM to about 500mM sucrose.

3. The method of claim 2, wherein the pharmaceutical composition comprises:
(i) from about 5 mM to about 20 mM phosphate; and/or
(ii) from about 70 mM to about 280 mM sucrose.

4. The method of claim 1, wherein the pharmaceutical composition comprises from about 0.01% to about 0.5% polyoxyethylene (20) sorbitan monooleate.

5. The method of claim 4, wherein the pharmaceutical composition comprises from about 0.01% to about 0.1% polyoxyethylene (20) sorbitan monooleate.

6. The method of claim 4, wherein the pharmaceutical composition comprises about 10 mM phosphate, about 210 mM sucrose, and about 0.05% polyoxyethylene (20) sorbitan monooleate.

7. The method of claim 1, wherein the phosphate is sodium phosphate.

8. The method of claim 1, wherein the pharmaceutical composition further comprises from about 1mM to about 500 mM proline.

9. The method of claim 1, wherein the pharmaceutical composition comprises a pH of about 7.3.

10. The method of claim 1, wherein the pharmaceutical composition is formulated at a concentration of the polypeptide from about 0.1 mg/ml to about 200 mg/mL.

11. The method of claim 1, wherein the pharmaceutical composition is formulated in a volume from about 0.1 mL to about 50 mL.

12. The method of claim 11, wherein the pharmaceutical composition is formulated at a concentration of the polypeptide of about 100 mg/mL in a volume of about 0.5 mL.

13. The method of claim 1, wherein the bone mineralization disorder is hypophosphatasia (HPP), bone fracture, osteoporosis, sclerosteosis, chondrocalcinosis, hypotonia, Duchenne's muscular dystrophy, tracheobronchomalacia, seizure, neurofibromatosis, and craniosynostosis.

14. The method of claim 1, wherein the polypeptide is administered at a dosage from about 0.01 mg/kg to about 20 mg/kg.

15. The method of claim 1, wherein the polypeptide is administered once per day, week, two weeks, month, or year.

16. The method of claim 1, wherein from about 10 to about 100 mg of the polypeptide is administered per week or every two weeks.

17. The method of claim 1, wherein about 15 mg, about 20 mg, about 30 mg, about 35 mg, about 45 mg, about 50 mg, or about 90 mg polypeptide is administered per week or every two weeks.

18. The method of claim 1, wherein the pharmaceutical composition is administered subcutaneously, intravenously, intramuscularly, sublingually, intrathecally, or intradermally.

19. The method of claim 18, wherein the pharmaceutical composition is administered subcutaneously or intravenously.

20. The method of claim 1, wherein the subject is a neonate, a child, an adolescent, or an adult human.


SEQUENCE LISTING (6 sequences)

SEQ ID NO: 1 - protein, 507 aa, Homo sapiens
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 2 - protein, 485 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANMGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKQKTDVEYESDEKARGTRLDGLDLVDTWKSFKPRYKHSHFIWNRTELLTLDPHNVDYLLGLFEPGDMQYELNRNQVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASS

SEQ ID NO: 3 - protein, 491 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANMGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKQKTDVEYESDEKARGTRLDGLDLVDTWKSFKPRYKHSHFIWNRTELLTLDPHNVDYLLGLFEPGDMQYELNRNQVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAA

SEQ ID NO: 4 - protein, 223 aa, synthetic construct
VECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 724 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANMGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKQKTDVEYESDEKARGTRLDGLDLVDTWKSFKPRYKHSHFIWNRTELLTLDPHNVDYLLGLFEPGDMQYELNRNQVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDDDDDDDDDD

SEQ ID NO: 6 - protein, 726 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD